(19) United States
(12) Patent Application Publication
(10) Pub. No.: US 2022/0056135 A1
(43) Pub. Date: Feb. 24, 2022

(54) BIFUNCTIONAL ANTI-PD-1/SIRPA MOLECULE

(71) Applicant: OSE IMMUNOTHERAPEUTICS, NANTES (FR)

(72) Inventors: NICOLAS POIRIER, TREILLIERES (FR); CAROLINE MARY, SAINTE PAZANNE (FR); AURORE MORELLO, SAINT SEBASTIEN SUR LOIRE (FR); KEVIN BITEAU, SAINTE PAZANNE (FR)

(21) Appl. No.: 17/414,971

(22) PCT Filed: Dec. 17, 2019
(86) PCT No.: PCT/EP2019/085785

§ 371 Date: Jun. 17, 2021

(30) Foreign Application Priority Data
Dec. 21, 2018 (EP) 18306810.5

(51) Int. Cl. C07K 16/28; C07K 14/705; A61K 39/395; A61K 38/17; A61K 45/06; A61P 35/00

(52) U.S. Cl. CPC C07K 16/2818; C07K 14/70596; A61K 39/3955; A61K 2039/505; A61K 45/06; A61P 35/00; A61K 38/1774

(57) ABSTRACT

The present invention relates to a bifunctional molecule comprising an anti-PD-1 antibody and SIRPa and its uses.

### FIELD OF THE INVENTION

The invention pertains to the field of immunotherapy. The present invention provides a bifunctional molecule that comprises an anti-PD1 antibody or antibody fragment thereof linked to SIRPa and uses thereof.

### BACKGROUND OF THE INVENTION

The approach of targeting T cell inhibition checkpoints for dis-inhibition with therapeutic antibodies is an area of intense investigation (for a review, see Pardoll, Nat Rev Cancer. 2012; 12:253-264). Targeting immune checkpoints of the adaptive immunity has shown great therapeutic efficacy to fight numerous cancers, but in a limited proportion of patients. Immune checkpoint on innate myeloid cells (macrophages, dendritic cells, MDSC, PMN) remain poorly studied while these cells represent the most abundant immune cell type in many solid tumors and are often associated with a poor outcome. Combining immune checkpoint therapies targeting both innate (mediated by myeloid cells) and adaptive (mediated by T cells) immune responses has demonstrated great efficiency in preclinical models but remains a challenge in clinic.

Immune cells activation is governed by the integration of balance co-stimulatory and co-inhibitory signals. T cell receptor (TCR)-mediated T cell activation is modulated by both co-stimulatory and co-inhibitory signals. The antigen-independent second signal modifies first signal, provided by interaction of antigenic peptide-MHC complex with the TCR, which confers specificity to the response. T cell co-stimulatory and co-inhibitory pathways have a broad immunoregulatory functions, controlling effector, memory and regulatory T cells, as well as naïve T cells. Therapeutic modulation of those pathways is translating to effective new strategies for treating cancer (For review, see Schildberg et al., 44(5), Immunity, 2016). Ongoing studies on regulation of the immune responses have led to the identification of multiple immunologic pathways that may be targeted for the development of cancer therapies. Those molecules are referred herein as immune checkpoint co-activators or co-inhibitors (see review Sharma et al., Cell, 161(2), 2015 and Pardoll, Nature Reviews Cancer, 12(4), 2012).

Programmed cell death protein 1 (PD-1, also known as CD279) is a cell surface protein molecule that belongs to the immunoglobulin superfamily. It is expressed on T and B lymphocytes and macrophages, and plays a role in cell fate and differentiation. Particularly, PD-1, functioning as an immune checkpoint, plays an important role in down-regulating the immune system by preventing the activation of T-cells, which in turn reduces autoimmunity and promotes self-tolerance. Two ligands for PD-1 have been identified, PD-L1 and PD-L2, that have been shown to downregulate T cell activation upon binding to PD-1 (Freeman et al. (2000) J Exp Med 192: 1027-34; Latchman et al. (2001) Nat Immunol 2:261-8; Carter et al. (2002) Eur J Immunol 32:634-43). The interaction between PD-1 and its ligand results in a decrease in tumor infiltrating lymphocytes, a decrease in T-cell receptor mediated proliferation, and immune evasion by the cancerous cells. Particularly, PD1 ligation reduces signals downstream of TCR stimulation on T cells, inhibiting T cell response and resulting in decreased activation and cytokine production.

Both strategies using anti-PD1 and anti-PDL1 inhibitor to disrupt their interaction was a success in cancer therapy (Brahmer et al., N Eng J Med, 366(26), 2012; Powles et al., Nature, 515(7528), 2014; Topalian et al., N Eng J Med, 366(26), 2012; Ansell, Curr Opin Hematol, 22(4), 2015). However, the accumulation of immunosuppressive and hypo-stimulatory myeloid cells within tumor micro-environment limits the efficiency of T-cell responses and the efficacy of immunotherapies, in particular those targeting at immune checkpoint such as PD-1/PD-L1. For example, low or no clinical response were seen in pancreatic cancer, non MSI colorectal cancer, gastric cancer and some breast cancer subtypes (Brahmer et al., N Engl J Med. 2012 Jun. 28; 366(26):2455-65, Feng et al., Cancer Lett. 2017 Oct. 28; 407:57-65, Borcherding et al., J Mol Biol. 2018 Jul. 6; 430(14):2014-2029). Multiple mechanisms have been described and may explain this low efficacy and resistance to PD-1/PD-L1 checkpoint therapy, such as (1) impaired formation of memory T cells, (2) impaired T cell infiltration, (3) insufficient generation of tumor specific T cells, (4) inadequate function of T cells, and (5) immunosuppressive microenvironment induced by regulatory T cells.

To date, the majority of therapies have focused on stimulating the adaptive immune system to attack cancer, including agents targeting PD-1/PD-L1 axis to rescue exhausted T cells and restore anti-tumor responses (Brahmer et al., N Eng J Med, 366(26), 2012; Topalian et al., N Eng J Med, 366(26), 2012; Wolchok et al., N Engl J Med. 2013 Jul. 11; 369(2): 122-133). However, macrophages and other myeloid immune cells also offer promise as effectors of cancer immunotherapy. The CD47/signal regulatory protein alpha (SIRPα) axis is a critical regulator of myeloid cell activation and serves a broad role as a myeloid-specific immune checkpoint.

Signal regulatory protein alpha, or SIRPa (also designated as SIRPα, CD172a or SHPS-1), is expressed on monocytes, most subpopulations of tissue macrophages, granulocytes, subsets of dendritic cells in lymphoid tissues, some bone marrow progenitor cells, and to varying levels on neurons, with a notably high expression in synapse-rich areas of the brain. Interaction of SIRPa, expressed by myeloid cells, with the ubiquitous CD47 that is overexpressed in some cancer cells but also widely expressed at lower levels by most healthy cells, is another important immune checkpoint of the innate response, involved in the regulation of myeloid functions. CD47 interacts with SIRPa and leads to the transmission of a “don't eat me” signal to phagocytic macrophages, which then leave target cells and potentially tumor cells unaffected. Blockade of the CD47/SIRPa pathway via agents targeting CD47, by enhancing antibody-dependent phagocytosis by macrophages, has been described to synergize with depleting therapeutic anticancer antibodies, to stimulate phagocytosis of cancer cells in vitro and to stimulate anti-tumor immune responses in vivo in a diverse range of preclinical models.

### SUMMARY OF THE INVENTION

To increase the efficacy of anti-PD1 immunotherapy and overcome potential anti PD-1 resistance in patient, the development of a combination treatment also targeting SIRPα/CD47 may be a good strategy. There remains therefore a significant need in the art for new and improved agents for safe immunotherapy, notably against cancer, targeting innate myeloid immune cells with an effective positive impact on adaptive immune response, in particular T cell immune responses. The present inventors have made a significant step forward with the invention disclosed herein. Strong benefic and unexpected effects are shown and explained notably at the beginning of the detailed description and in the examples. The inventors provide a bifunctional molecule comprising an anti-hPD-1 antibody and a human SIRPα promising for numerous therapeutic applications, in particular for the treatment of cancer. The present invention is based on the development of an antibody specifically targeting human PD-1 which shows high binding affinity to PD-1 and strong competition with its ligands PDL-1 and PD-L2. Surprisingly, the fusion of the N-terminal end of SIRPa to the C-terminal end of the Fc region of an anti-hPD-1 antibody allows the conservation of its high affinity for CD47 (the SIRPa ligand) to similar extend to endogenous SIRPa. The fusion of the Fc domain to SIRPa also increases the product half-life. Furthermore, the bifunctional anti-PD1-SIRPa molecule disclosed herein potentiates activation of T cells (NFAT mediated activation) compared to anti PD-1 alone. Particularly, the anti-PD1-SIRPa bifunctional molecule induces the proliferation and activation of naïve, partially exhausted subsets reflected by cytokine (e.g. IFNγ) secretion. The bifunctional anti-PD1-SIRPa molecule shows a surprising synergistic effect. Such anti-hPD1-SIRPa bifunctional molecule has the capacity to overcome associated resistance mechanism and improve efficacy of anti PD-1 immunotherapies.

In a first aspect, the invention concerns a bifunctional molecule that comprises:

(a) an anti-human PD-1 antibody or an antigen-binding fragment thereof, which comprises:
(i) a heavy chain variable domain (VH) comprising a HCDR1, a HCDR2 and a HCDR3, and
(ii) a light chain variable domain (VL) comprising a LCDR1, a LCDR2 and a LCDR3, and
   (b) a human SIRPa or a fragment or variant thereof,
   wherein the C-terminal end of the heavy and/or light chain(s) of the antibody or antigen-binding fragment thereof is covalently linked to the N-terminal end of the SIRPa or fragment or variant thereof as a fusion protein, preferably by a peptide linker.

Particularly, the antibody is a chimeric, a humanized or a human antibody.

Preferably, the SIRPa fragment comprises or consists of the extracellular domain of SIRPa. Even more preferably, the SIRPa fragment is devoid of the intracellular part thereof and optionally of the transmembrane domain thereof, preferably wherein the SIRPa comprises or consists of the amino acid sequence set forth in SEQ ID NO: 51 or a fragment thereof.

In a particular aspect, the invention relates to a bifunctional molecule comprises an anti-human PD-1 antibody or antigen-binding fragment thereof, that comprises or consists of:
(i) a heavy chain variable domain (VH) comprising HCDR1, HCDR2 and HCDR3, and
(ii) a light chain variable domain (VL) comprising LCDR1, LCDR2 and LCDR3,
   wherein:
the heavy chain CDR1 (HCDR1) comprises or consists of an amino acid sequence of SEQ ID NO: 1;
the heavy chain CDR2 (HCDR2) comprises or consists of an amino acid sequence of SEQ ID NO: 2;
the heavy chain CDR3 (HCDR3) comprises or consists of an amino acid sequence of SEQ ID NO: 3 wherein X1 is D or E and X2 is selected from the group consisting of T, H, A, Y, N, E and S, preferably in the group consisting of H, A, Y, N, and E;
the light chain CDR1 (LCDR1) comprises or consists of an amino acid sequence of SEQ ID NO: 12 wherein X is G or T;
the light chain CDR2 (LCDR2) comprises or consists of an amino acid sequence of SEQ ID NO: 15,
the light chain CDR3 (LCDR3) comprises or consists of an amino acid sequence of SEQ ID NO:16.

Particularly, the anti-human PD-1 antibody or antigen-binding fragment thereof, comprises or consists of: (a) a VH comprising or consisting of an amino acid sequence of SEQ ID NO: 17, wherein X1 is D or E and X2 is selected from the group consisting of T, H, A, Y, N, E and S preferably in the group consisting of H, A, Y, N and E; and (b) a VL comprising or consisting of an amino acid sequence of SEQ ID NO: 26, wherein X is G or T.

In a particular aspect, the antibody or antigen-binding fragment thereof comprises a light chain constant domain derived from a human kappa light chain constant domain and a heavy chain constant domain derived from a human IgG1, IgG2, IgG3 or IgG4 heavy chain constant domain, preferably an IgG1 or IgG4 heavy chain constant domain.

In a more specific aspect, the antibody or antigen-binding fragment thereof comprises a light chain constant domain derived from a human kappa light chain constant domain and a heavy chain constant domain derived from a human IgG1 heavy chain constant domain, optionally with a substitution or a combination of substitutions selected from the group consisting of T250Q/M428L; M252Y/S254T/T256E+H433K/N434F; E233P/L234V/L235A/G236A+A327G/A330S/P331S; E333A; S239D/A330L/I332E; P257I/Q311; K326W/E333S; S239D/I332E/G236A; N297A; L234A/L235A; N297A+M252Y/S254T/T256E; and K322A and K444A, preferably selected from the group consisting of N297A optionally in combination with M252Y/S254T/T256E, and L234A/L235A.

In another more specific aspect, the antibody or antigen-binding fragment thereof comprises a light chain constant domain derived from a human kappa light chain constant domain and a heavy chain constant domain derived from a human IgG4 heavy chain constant domain, optionally with a substitution or a combination of substitutions selected from the group consisting of S228P; L234A/L235A, S228P+M252Y/S254T/T256E and K444A.

Particularly, the anti-PD1 antibody is be selected from the group consisting of Pembrolizumab, Nivolumab, Pidilizumab, Cemiplimab, PDR001, and monoclonal antibodies 5C4, 17D8, 2D3, 4H1, 4A11, 7D3, and 5F4. In another aspect, the invention concerns, an isolated nucleic acid sequence or a group of isolated nucleic acid molecules encoding a bifunctional molecule as disclosed herein, a vector comprising a nucleic acid or group of nucleic acid molecules as disclosed herein, and/or a host cell, comprising the vector the nucleic acid or group of nucleic acid molecules as disclosed herein.

In another aspect, the invention relates to a method for producing the bifunctional molecule, comprising a step of culturing a host cell according as disclosed herein and optionally a step of isolating the bifunctional molecule.

In another aspect, the invention concerns a pharmaceutical composition comprising the bifunctional molecule, the nucleic acid or group of nucleic acid molecules, the vector or the host cell as disclosed herein and a pharmaceutically acceptable carrier.

Optionally, the pharmaceutical composition further comprises an additional therapeutic agent, preferably selected in the group consisting of alkylating agents, angiogenesis inhibitors, antibodies, antimetabolites, antimitotics, antiproliferatives, antivirals, aurora kinase inhibitors, apoptosis promoters (for example, Bcl-2 family inhibitors), activators of death receptor pathway, Bcr-Abl kinase inhibitors, BiTE (Bi-Specific T cell Engager) antibodies, antibody drug conjugates, biologic response modifiers, Bruton's tyrosine kinase (BTK) inhibitors, cyclin-dependent kinase inhibitors, cell cycle inhibitors, cyclooxygenase-2 inhibitors, DVDs, leukemia viral oncogene homolog (ErbB2) receptor inhibitors, growth factor inhibitors, heat shock protein (HSP)-90 inhibitors, histone deacetylase (HDAC) inhibitors, hormonal therapies, immunologicals, inhibitors of inhibitors of apoptosis proteins (IAPs), intercalating antibiotics, kinase inhibitors, kinesin inhibitors, Jak2 inhibitors, mammalian target of rapamycin inhibitors, microRNAs, mitogen-activated extracellular signal-regulated kinase inhibitors, multivalent binding proteins, non-steroidal anti-inflammatory drugs (NSAIDs), poly ADP (adenosine diphosphate)-ribose polymerase (PARP) inhibitors, platinum chemotherapeutics, polo-like kinase (Plk) inhibitors, phosphoinositide-3 kinase (PI3K) inhibitors, proteasome inhibitors, purine analogs, pyrimidine analogs, receptor tyrosine kinase inhibitors, retinoids/deltoids plant alkaloids, small inhibitory ribonucleic acids (siRNAs), topoisomerase inhibitors, ubiquitin ligase inhibitors, hypomethylating agents, checkpoints inhibitors, peptide vaccine and the like, epitopes or neoepitopes from tumor antigens, as well as combinations of one or more of these agents.

Particularly, the pharmaceutical composition, bifunctional molecule, nucleic acid or group of nucleic acid molecules, vector or host cell are for use as a medicament.

The invention finally relates to a pharmaceutical composition, a bifunctional molecule, a nucleic acid or group of nucleic acid molecules, a vector, or a host cell as disclosed herein for use as a medicament.

In a particular aspect, the pharmaceutical composition, bifunctional molecule, nucleic acid or group of nucleic acid molecules, vector, or host cell as disclosed herein for use in the treatment of cancer, preferably a cancer selected from the group consisting of a hematologic malignancy or a solid tumor with expression of PD-1 and/or PD-L1 such as a cancer selected from the group consisting of hematolymphoid neoplasms, angioimmunoblastic T cell lymphoma, myelodysplastic syndrome, and acute myeloid leukemia, a cancer induced by virus or associated with immunodeficiency such as a cancer selected from the group consisting of Kaposi sarcoma (e.g., associated with Kaposi sarcoma herpes virus); cervical, anal, penile and vulvar squamous cell cancer and oropharyngeal cancers (e.g., associated with human papilloma virus); B cell non-Hodgkin lymphomas (NHL) including diffuse large B-cell lymphoma, Burkitt lymphoma, plasmablastic lymphoma, primary central nervous system lymphoma, HHV-8 primary effusion lymphoma, classic Hodgkin lymphoma, and lymphoproliferative disorders (e.g., associated with Epstein-Barr virus (EBV) and/or Kaposi sarcoma herpes virus); hepatocellular carcinoma (e.g., associated with hepatitis B and/or C viruses); Merkel cell carcinoma (e.g., associated with Merkel cell polyoma virus (MPV)); and cancer associated with human immunodeficiency virus infection (HIV) infection, and a cancer selected from the group consisting of metastatic or not metastatic, Melanoma, malignant mesothelioma, Non-Small Cell Lung Cancer, Renal Cell Carcinoma, Hodgkin's Lymphoma, Head and Neck Cancer, Urothelial Carcinoma, Colorectal Cancer, Hepatocellular Carcinoma, Small Cell Lung Cancer, Metastatic Merkel Cell Carcinoma, Gastric or Gastroesophageal cancers and Cervical Cancer; or infectious disease, preferably chronic infectious disease, even more preferably chronic viral infections, preferably caused by a virus selected from the group consisting of HIV, hepatitis virus, herpes virus, adenovirus, influenza virus, flaviviruses, echovirus, rhinovirus, coxsackie virus, coronavirus, respiratory syncytial virus, mumps virus, rotavirus, measles virus, rubella virus, parvovirus, vaccinia virus, HTLV virus, dengue virus, papillomavirus, molluscum virus, poliovirus, rabies virus, JC virus and arboviral encephalitis virus.

Preferably, the cancer is a PD-1, a PD-L1 and/or a PD-L2 positive cancer, in particular a PD-L1 positive cancer.

Optionally, the bifunctional molecule, the pharmaceutical composition, the isolated nucleic acid molecule or the group of isolated nucleic acid molecules, the vector, or the host cell is for use in combination with radiotherapy or an additional therapeutic agent, preferably selected in the group consisting of alkylating agents, angiogenesis inhibitors, antibodies, antimetabolites, antimitotics, antiproliferatives, antivirals, aurora kinase inhibitors, apoptosis promoters (for example, Bcl-2 family inhibitors), activators of death receptor pathway, Bcr-Abl kinase inhibitors, BiTE (Bi-Specific T cell Engager) antibodies, antibody drug conjugates, biologic response modifiers, Bruton's tyrosine kinase (BTK) inhibitors, cyclin-dependent kinase inhibitors, cell cycle inhibitors, cyclooxygenase-2 inhibitors, DVDs, leukemia viral oncogene homolog (ErbB2) receptor inhibitors, growth factor inhibitors, heat shock protein (HSP)-90 inhibitors, histone deacetylase (HDAC) inhibitors, hormonal therapies, immunologicals, inhibitors of inhibitors of apoptosis proteins (IAPs), intercalating antibiotics, kinase inhibitors, kinesin inhibitors, Jak2 inhibitors, mammalian target of rapamycin inhibitors, microRNAs, mitogen-activated extracellular signal-regulated kinase inhibitors, multivalent binding proteins, non-steroidal anti-inflammatory drugs (NSAIDs), poly ADP (adenosine diphosphate)-ribose polymerase (PARP) inhibitors, platinum chemotherapeutics, polo-like kinase (Plk) inhibitors, phosphoinositide-3 kinase (PI3K) inhibitors, proteasome inhibitors, purine analogs, pyrimidine analogs, receptor tyrosine kinase inhibitors, retinoids/deltoids plant alkaloids, small inhibitory ribonucleic acids (siRNAs), topoisomerase inhibitors, ubiquitin ligase inhibitors, hypomethylating agents, checkpoints inhibitors, peptide vaccine and the like, epitopes or neoepitopes from tumor antigens, as well as combinations of one or more of these agents.

In one aspect, the pharmaceutical composition, bifunctional molecule, nucleic acid or group of nucleic acid molecules, vector or host cell as disclosed herein are for use for inhibiting of suppressive activity of T regulator cells, activating of T effector cells and/or stimulating proliferation of naïve partially exhausted T-cells.

### BRIEF DESCRIPTION OF THE DRAWINGS

FIG. 1: PD-1 binding ELISA assay of anti PD-1 antibody versus anti PD-1/SIRPa bifunctional molecule. Human recombinant PD-1 protein was immobilized and anti-PD-1 bifunctional molecules were added at different concentrations. Revelation was performed with an anti-human Fc antibody coupled to peroxidase. Colorimetry was determined at 450 nm using TMB substrate. (A) Data of anti-PD1VH-SIRPa antibody chimeric (●) versus humanized (▪). (B) Data of anti-PD1VL-SIRPa antibody chimeric (●) versus humanized form (▪). In this experiment, the bifunctional molecule comprises a humanized anti-PD1 antibody having a heavy chain variable domain as disclosed in SEQ ID NO:18 and a light chain variable domain as disclosed in SEQ ID NO:28.

FIG. 2: Anti PD-1/SIRPa bifunctional molecules block PD1/PDL1 interaction. Competition PD-1/PD-L1 ELISA assay. PD-L1 is immobilized and complex antibody+biotinylated recombinant human PD-1 was added. Different concentrations of Anti-PD-1 antibody were tested and rec PD1 was added at 0.6 μg/mL. Anti-PD-1 antibody (▪) or Bicki anti-PD1VH-Sirpa (●) or anti-PD1VL-Sirpa (∘) antibodies were added at different concentrations. Revelation was performed with streptavidin peroxidase to detect PD1 molecule and revealed by colorimetry at 450 nm using TMB substrate. In this experiment, the bifunctional molecule comprises the chimeric anti-PD1 antibody comprising a heavy chain as defined in SEQ ID NO: 53 and a light chain as defined in SEQ ID NO: 54.

FIG. 3: Bridging ELISA Binding assay. PD1-His recombinant protein was immobilized and Bicki anti-PD1VHSirpa (●) or anti-PD1VLSirpa (∘) were added at serial concentrations. CD47 Fc recombinant protein was then added at 1 μg/mL. Detection was performed with an anti CD47 mouse antibody (clone B6H12)+an anti IgG mouse antibody coupled to peroxidase. ELISA was revealed by colorimetry at 450 nm using TMB substrate. Histogram represents recombinant rSIRPa protein immobilized on the plate and was used as positive control for ELISA. In this experiment, the bifunctional molecule comprises the chimeric anti-PD1 antibody comprising a heavy chain as defined in SEQ ID NO: 53 and a light chain as defined in SEQ ID NO: 54.

FIG. 4: Targeting and binding of Bicki anti-PD1-SIRPa molecules on PD1+CD47+ expressing T cells. Jurkat cells expressing CD47+ only (grey bar) or co-expressing CD47+ and PD-1+(black bar) were stained with 4.5 nM of BiCKi anti PD-1 SIRPa or SIRPa-Fc and revealed with an anti IgG-PE (Biolegend, clone HP6017). Data represent ratio of the Median fluorescence on PD-1+CD47+ Jurkat cells over the Median fluorescence obtained on PD1-cells CD47+ Jurkat cells. In this experiment, the bifunctional molecule comprises a humanized anti-PD1 antibody having a heavy chain variable domain as disclosed in SEQ ID NO:24 and a light chain variable domain as disclosed in SEQ ID NO:28.

FIG. 5: Bicki anti-PD1-Sirpa molecules synergistically potentiate T cell activation in vitro by stimulating NFAT signaling. A promega PD-1/PD-L1 bioassay was performed for this experiment to determine T cell activation using NFAT luciferase reporter system. Two cell lines are used (1) Effector T cells (Jurkat stably expressing PD-1, NFAT-induced luciferase) and (2) activating target cells (CHO K1 cells stably expressing PDL1 and surface protein designed to activate cognate TCRs in an antigen-independent manner). When cells are cocultured, PD-L1/PD-1 interaction directly inhibits TCR mediated activation thereby blocking NFAT activation and luciferase activity. The addition of an anti-PD1 antibody blocks the inhibitory signal leading to NFAT activation and luciferase synthesis. After adding BioGlo™ luciferin, Luminescence is quantified using a luminometer and reflects T cell activation. (A) PD-1 and CD47 expression on effector reporter T cell line (anti-PD-1 Pecy7, BD Bioscience clone EH12.2; Anti CD47 (clone B6H12)+anti mouse IgG AF647. (B) Serial dilution of anti-PD1 antibody alone (▾) or bicki anti-PD1VH-SIRPa antibody (♦) or isotype control (▪), anti PD-1 antibody+isotype control VH-SIRPa antibody (●) or anti-PD1 antibody+SIRPa Fc (◯) were tested. (C) Effector Jurkat cells were preincubated with anti CD47 blocking antibody (B6H12) (●) or without blocking antibody (♦), then incubated with different concentration of Bicki anti-PD1VH-SIRPa. As baseline activation, Jurkat cells were also incubated with anti PD-1 antibody alone (▾). (D) The efficacy of Bicki anti-PD1VH-SIRPa constructed with IgG4 S228P (♦) or IgG1 N298A (▪) was assessed and compared to the anti PD-1 alone (▾). (E) In another experiment, synergistic activity of the Bicki VH SIRPa (◯)) versus antibody anti PD-1 alone (●) was tested with other anti PD-1 backbones: pembrolizumab (left graph) and nivolumab (right graph). (F) Another bicki anti-PD1-Type I protein antibody (●) was tested and compared to an anti-PD1 antibody (▪). In this experiment, the bifunctional molecule comprises a humanized anti-PD1 antibody having a heavy chain variable domain as disclosed in SEQ ID NO:24 and a light chain variable domain as disclosed in SEQ ID NO:28.

FIG. 6: Bicki anti-PD1-SIRPa molecules potentiate calcium flux signaling in T cell to similar extend to CD28 co-stimulation. CD47+ PD1+ Jurkat cells were stained with Fura-red to measure intracellular Ca2+ release following activation using flow cytometry. T cells were stimulated with BiCKI SIRPa alone (◯ grey line) or in combination with CD3 (OKT3) stimulation (◯ black line). As control, cells were stimulated with a-CD3 only (●) or with a-CD3+CD28 (▪). (A) Graph represents the mean of 4 to 6 experiments and the arrow illustrated the addition of the stimuli. Data were obtained by calculating the ratio BV711 (linked Ca2+)/PercyP5.5 5 (free Ca2+) MFI. This ratio was normalized to the unstimulated (mean of the 20 first second before stimulation). (B) Data represent the Area under the curve (AUC) calculated; each dot represents one experiment. p value was calculated using paired t test (*p<0,05). In this experiment, the bifunctional molecule comprises a humanized anti-PD1 antibody having a heavy chain variable domain as disclosed in SEQ ID NO:24 and a light chain variable domain as disclosed in SEQ ID NO:28.

FIG. 7: Bicki anti-PD1-SIRPa molecules stimulate proliferation of PBMCs and secretion of IFNg. (A) PBMCs were isolated from 3 healthy donors and activated with an immobilized CD3 antibody (OKT3.3 μg/mL) in the presence of an isotype control, an anti-PD1 antibody or Bicki anti-PD1VH-SIRPa or anti-PD1VL-SIRPa. Proliferation was assessed by thymidine incorporation 3H on Day 6. (B) PBMCs isolated from healthy donor were activated with immobilized anti CD3 antibody (OKT23 1 ug/mL) and anti-CD28 antibody (clone CD28.2) in the presence of the anti-PD-1 antibody alone, anti-PD1+rSIRPa protein, Bicki anti-PD1VH-Sirpa or anti-PD1VL-Sirpa. Day 2 following activation. Supernatant was harvested and IFNg was dosed by ELISA. Data are representatives of 3 different donors. In this experiment, the bifunctional molecule comprises a humanized anti-PD1 antibody having a heavy chain variable domain as disclosed in SEQ ID NO:24 and a light chain variable domain as disclosed in SEQ ID NO:28.

FIG. 8: Bicki anti-PD1-SIRPa molecules enhance activated T cell proliferation and IFNg secretion. CD3 CD28 pre-activated T cells were re-stimulated on CD3/PDL1 coated plate in the presence of the anti-PD1VH-SIRPa or the anti-PD1VL-SIRPa. (10 ug/mL). Anti-PD-1 antibody and isotype antibody are used as control. (A) T cell proliferation was assessed by H3 thymidine incorporation at Day 6. (B) Supernatant was collected on Day 6 and IFNg secretion was dosed by ELISA. Following activation, supernatant was harvested and IFNg was dosed by ELISA. Data are representatives of 3 different donors. In this experiment, the bifunctional molecule comprises a humanized anti-PD1 antibody having a heavy chain variable domain as disclosed in SEQ ID NO:24 and a light chain variable domain as disclosed in SEQ ID NO:28.

FIG. 9: Bicki anti-PD1-SIRPa bifunctional molecule enhance proliferation of exhausted T cells. Human PBMCs were repeatedly stimulated on CD3 CD28 coated plate (3 ug/mL of OKT3 and 3 μg/mL CD28.2 antibody) every 3 days. After the 3rd stimulations, T cells were reactivated on CD3/PD-L1 coated plate and incubated with an isotype control or an anti-PD1, a rSIRPa protein or a Bicki anti-PD1-VH-Sirpa antibody. H3 incorporation assay was performed on Day 5 to determine T cell proliferation. Data are expressed in fold change and are representative of 3 different donors (1=isotype control). In this experiment, the bifunctional molecule comprises a humanized anti-PD1 antibody having a heavy chain variable domain as disclosed in SEQ ID NO:24 and a light chain variable domain as disclosed in SEQ ID NO:28.

FIG. 10: Bicki anti-PD1-SIRPa bifunctional molecule enhance T cell migration into the tumor 3D multicellular Spheroids were generated by coculturing A549 tumor cells, MRC-5 fibroblasts and human monocytes in low attachment plates. On Day 3, human T cells were added to the well and 72 hours following coculture, T cell infiltration was analyzed by immunofluorescence (anti-human CD3+anti-Donkey A488 secondary antibody) and all cells were stained with DAPI nuclear marker. Fluorescence signals were quantified by confocal microscopy and analyzed using FIJI software. Data represents the number of CD3+ positives cells infiltrated into the spheroid/1e6 the DAPI+ total cells and each dot represents the analysis of one spheroid. In this assay, spheroids were treated with isotype control (IgG4) or biCKI SIRPa (50 nM) for 3 days. Statistical significance was determined using a Mann Whitney test *p<0,05. In this experiment, the bifunctional molecule comprises a humanized anti-PD1 antibody having a heavy chain variable domain as disclosed in SEQ ID NO:24 and a light chain variable domain as disclosed in SEQ ID NO:28.

FIG. 11: Pharmacokinetics of Bicki anti-PD1-SIRPa bifunctional molecule in mice. Mice were intravenously injected with one dose (34.34 nM/kg) of anti PD-1 SIRPa constructed with an IgG1N298A (◯) or with IgG4 S228P isotype (●). Concentration of the Bicki in the serum was assessed by a sandwich ELISA at multiple time points following injection. In this experiment, the bifunctional molecule comprises a humanized anti-PD1 antibody having a heavy chain variable domain as disclosed in SEQ ID NO:24 and a light chain variable domain as disclosed in SEQ ID NO:28.

FIG. 12: Illustration of the mechanism of action of the Bicki anti-PD1-SIRPa bifunctional molecule in comparison to the prior art. Left part of the Figure illustrates the mechanism of anti-PD-L1-SIRPa bifunctional molecules of the prior art targeting the cancer cells. Right part of the Figure illustrates the mechanism of anti-PD-1-SIRPa bifunctional molecules of the invention which targets T cells, especially the same T cell, and thereby have the capacity to synergistically reactivate exhausted T cells.

### DETAILED DESCRIPTION OF THE INVENTION

### Introduction

The antibodies of the invention are bifunctional since they combine the specific anti-PD-1 effects and the effects of SIRPa fused to the anti-PD-1 antibody. Indeed, the present invention relates to a bifunctional molecule comprising an anti-PD-1 antibody and SIRPa, the protein SIRPa being covalently linked to a polypeptide chain of the anti-PD-1 antibody, either the light chain or the heavy chain of the antibody or both or any fragment thereof. The chain of the anti-PD-1 antibody or a fragment thereof and the SIRPa are prepared as a fusion protein. In this particular aspect, the N terminal end of SIRPa is linked to the C terminal end of the chain of the anti-PD-1 antibody or a fragment thereof, optionally through a peptide linker.

Firstly, it is emphasized that SIRPa is known in the prior art to be the target of anti-SIRPa antibodies. These antibodies block the interaction between CD47 on tumor cells and SIRPa on myeloid cells, in particular macrophages; said interaction (in a so-called Trans interaction, between different cells) inducing an inhibition of phagocytosis of tumor cells by the macrophages. On the opposite, the bifunctional protein of the invention comprising SIRPa and an antibody against a ligand expressed on T cell, especially PD-1, was not known to be involved in a T cell activation mechanism.

As explained and shown in detail, notably the examples of the present applications, and as illustrated in FIG. 12, the inventors have obtained bifunctional SIRPa-anti-PD-1 molecules that unexpectedly have the strong benefit of targeting on the same T cell both:
PD-1 on a T cell: anti-PD1 antibody part of the bifunctional SIRPa-anti-PD-1 molecules; and
CD47 (not or not only CD47 on tumoral cell): SIRPa part of the bifunctional SIRPa-anti-PD-1 molecules.
   This double targeting on a same T cell leads to a previously unknown synergistic effect as shown by the supplementary activation of the NFAT pathway. This capacity is of particular interest for the following reasons.

As known by the one skilled in the art, tumoral cells are not sufficiently eliminated by T cells in relation with an exhaustion of T cells. Anti PD-1 therapeutic compounds are used clinically in order to activate T cells through the inhibition of the inhibiting effect of PD1-PDL1 interaction (PD1 on T cells and PDL1 on tumoral cells). More precisely, T cell exhaustion is observed in humans with cancer. As described for instance in Jiang, Y., Li, Y. and Zhu, B (Cell Death Dis 6, e1792 (2015)), the exhausted T cells in the tumor microenvironment show overexpressed inhibitory receptors, decreased effector cytokine production and cytolytic activity, leading to the failure of cancer elimination. Restoring exhausted T cells represents a clinical strategy for cancer treatment. Most T cells in tumor microenvironment are exhausted, leading to cancer immune evasion. PD-1 is the major inhibitory receptor regulating T-cell exhaustion and T cells with high PD-1 expression lose the ability to eliminate cancer. Anti-PD-1 antibodies are not always sufficiently efficient to allow the «re»activation of exhausted T cells. Therefore, this is an important medical need at the present time. The inventors have shown that the bifunctional anti-PD1-SIRPa molecule disclosed herein potentiates activation (NFAT mediated activation, calcium release) of T cells, in particular exhausted T cells, compared to anti PD-1 alone or combined with SIRPa. Particularly, the anti-PD1-SIRPa bifunctional molecule induces the proliferation and activation of naïve, partially exhausted T-cell subsets as reflected by cytokine (e.g. IFNγ) secretion. Such anti-hPD1-SIRPa bifunctional molecule has the capacity to overcome associated resistance mechanism and to improve efficacy of anti PD-1 immunotherapies.

Applicant also shows that the interaction of the bifunctional anti-PD1-SIRPa molecules, with a single T cell expressing i) PD1 and ii) SIRPa receptor, leads to the unexpected activation of the NFAT pathway (TCR signaling) with a positive effect on T cells activation, in particular exhausted T cells, favorizing the capacity of T cells to eliminate tumoral cells.

It means that, on one side, SIRPa of the BICKI molecule targets SIRPa receptors (CD47), activating this pathway, like SIRPa alone, and on the other side, the anti-PD1 part of the BICKI molecule blocks PD-1. The bifunctional molecules target both CD47 and PD-1 on the same cells. This results in a synergistic activation of the TCR (NFAT) signaling that has never been observed using a combination of anti-PD1 antibody and SIRPa separately (administration of antiPD1 and of SIRPa as two separate compounds). For instance, this activation by acting on the same cell cannot be provided by bifunctional molecule that targets PD-L1. Indeed, it is known in the art that PD-L1 is mainly expressed on tumoral cells and not on immune cells such as T cells.

In addition, the synergistic effect has been observed with the particular humanized anti-PD-1 of the invention but also with two others anti-PD-1 of reference, namely pembrolizumab and nivolumab. The design of the bifunctional anti-PD1-SIRPa molecules allows to target CD47+PD-1+ exhausted T cells over other CD47+ cells at least by a factor 2. The bifunctional anti-PD1-SIRPa molecules potentiate anti-PD1 effect and strongly suggest that SIRPa binding on CD47 not only blocks “don't EAT-ME” inhibitory phagocytic signals but also surprisingly promotes CD47-dependent T-cell costimulation. Finally, bifunctional anti-PD1-SIRPa molecules enhance the migration of the T cells into the tumor microenvironment, thereby overcoming one of the major resistance mechanisms associated to the anti PD-1 monotherapy due to a lack of T cell into the tumor microenvironment.

The bifunctional molecules of the invention have in particular one or several of the following advantages:
They conserve their capacity to bind PD-1 without differences between Humanized and chimeric form of the anti-PD1 antibody.
They conserve their capacity to bind CD47, SIRPa ligand protein.
They antagonize PD-1/PD-L1 and/or PD-1/PD-L2 interaction.
They synergistically potentiate activation of T cells (NFAT mediated activation and calcium flux stimulation) compared to anti PD-1 alone.
They demonstrate a synergistic effect to stimulate proliferation of naïve, activated and exhausted T cells.
They demonstrate a synergistic effect to stimulate secretion of IFNg by T cells.
They potentiate migration of T cells.
They demonstrate a good and linear pharmacokinetics profile.

### Definitions

In order that the present invention may be more readily understood, certain terms are defined hereafter. Additional definitions are set forth throughout the detailed description.

Unless otherwise defined, all terms of art, notations and other scientific terminology used herein are intended to have the meanings commonly understood by those of skill in the art to which this invention pertains. In some cases, terms with commonly understood meanings are defined herein for clarity and/or for ready reference, and the inclusion of such definitions herein should not necessarily be construed to represent a difference over what is generally understood in the art. The techniques and procedures described or referenced herein are generally well understood and commonly employed using conventional methodologies by those skilled in the art.

As used herein, the terms “Signal-regulatory protein alpha”, “SIRPα” and “SIRPa” refers to a receptor-type transmembrane glycoprotein that is mammalian Immunoglobulin-like cell surface receptor for CD47. It particularly refers to SIRPa polypeptides, derivatives and analogs thereof having substantial amino acid sequence identity to wild-type mammalian SIRPa and substantially equivalent biological activity, e.g., in standard bioassays or assays of SIRPa receptor binding affinity. For example, SIRPa refers to an amino acid sequence of a recombinant or non-recombinant polypeptide having an amino acid sequence of: i) a native or naturally-occurring allelic variant of a SIRPa polypeptide, ii) a biologically active fragment of a SIRPa polypeptide, iii) a biologically active polypeptide analog of a SIRPa polypeptide, or iv) a biologically active variant of a SIRPa polypeptide. The SIRPa can comprise its transmembrane domain and/or cytoplasmic domain or be devoid of it. The SIRPa preferably comprises or consists in its extracellular domain. Alternative designations for this molecule are “Tyrosine-protein phosphatase non-receptor type substrate 1” and “SHP substrate 1”, “CD172 antigen-like family member A”, “p84” and “Macrophage fusion receptor”. Preferably, the term “SIRPa” refers to human SIRPa. For example, the human SIRPa amino acid sequence is about 504 amino acids and has a Genbank accession number of NP_001035111.1, NP_001035112.1, NP_001317657.1, or NP_542970.1. Preferably, the human SIRPa is the isoform 1. Even more preferably, the SIRPa essentially consists in the amino-acids of positions 31-373 of the aforementioned sequences, i.e. its extracellular domain. Human SIRPa is described in UniProtKB—P78324. As used herein, the terms “Programmed Death 1”, “Programmed Cell Death 1”, “PD1”, “PD-1”, “PDCD1”, “PD-1 antigen”, “human PD-1”, “hPD-1” and “hPD-1” are used interchangeably and refer to the Programmed Death-1 receptor, also known as CD279, and include variants and isoforms of human PD-1, and analogs having at least one common epitope with PD-1. PD-1 is a key regulator of the threshold of immune response and peripheral immune tolerance. It is expressed on activated T cells, B cells, monocytes, and dendritic cells and binds to its ligands PD-L1 and PD-L2. Human PD-1 is encoded by the PDCD1 gene. As an example, the amino acid sequence of a human PD-1 is disclosed under GenBank accession number NP_005009. PD1 has four splice variants expressed on human Peripheral blood mononuclear cells (PBMC). Accordingly, PD-1 proteins include full-length PD-1, as well as alternative splice variants of PD-1, such as PD-1Aex2, PD-1Aex3, PD-1Aex2,3 and PD-1Aex2,3,4. Unless specified otherwise, the terms include any variant and isoform of human PD-1 that are naturally expressed by PBMC, or that are expressed by cells transfected with a PD-1 gene.

As used herein, the term “antibody” describes a type of immunoglobulin molecule and is used in its broadest sense. In particular, antibodies include immunoglobulin molecules and immunologically active fragments of immunoglobulin molecules, i.e., molecules that contain an antigen binding site. Immunoglobulin molecules can be of any type (e.g., IgG, IgE, IgM, IgD, IgA and IgY), class (e.g., IgG1, IgG2, IgG3, IgG4, IgA1 and IgA2) or subclass. The heavy-chain constant domains that correspond to the different classes of immunoglobulins are called alpha, delta, epsilon, gamma, and mu, respectively. Unless specifically noted otherwise, the term “antibody” includes intact immunoglobulins and “antibody fragment” or “antigen binding fragment” (such as Fab, Fab′, F(ab′)2, Fv), single chain (scFv), mutants thereof, molecules comprising an antibody portion, diabodies, linear antibodies, single chain antibodies, and any other modified configuration of the immunoglobulin molecule that comprises an antigen recognition site of the required specificity, including glycosylation variants of antibodies, amino acid sequence variants of antibodies. Preferably, the term antibody refers to a humanized antibody.

As used herein, an “antigen-binding fragment” of an antibody means a part of an antibody, i.e. a molecule corresponding to a portion of the structure of the antibody of the invention, that exhibits antigen-binding capacity for PD-1, possibly in its native form; such fragment especially exhibits the same or substantially the same antigen-binding specificity for said antigen compared to the antigen-binding specificity of the corresponding four-chain antibody. Advantageously, the antigen-binding fragments have a similar binding affinity as the corresponding 4-chain antibodies. However, antigen-binding fragment that have a reduced antigen-binding affinity with respect to corresponding 4-chain antibodies are also encompassed within the invention. The antigen-binding capacity can be determined by measuring the affinity between the antibody and the target fragment. These antigen-binding fragments may also be designated as “functional fragments” of antibodies. Antigen-binding fragments of antibodies are fragments which comprise their hypervariable domains designated CDRs (Complementary Determining Regions) or part(s) thereof encompassing the recognition site for the antigen, i.e. the extracellular domain of PD1, thereby defining antigen recognition specificity.

A “Fab” fragment contains the constant domain of the light chain and the first constant domain (CH1) of the heavy chain. Fab′ fragments differ from Fab fragments by the addition of a few residues at the carboxyl terminus of the heavy chain CH1 domain including one or more cysteines from the antibody hinge region. F(ab′) fragments are produced by cleavage of the disulfide bond at the hinge cysteines of the F(ab′)2 pepsin digestion product. Additional chemical couplings of antibody fragments are known to those of ordinary skill in the art. Fab and F(ab′)2 fragments lack the Fc fragment of an intact antibody, clear more rapidly from the circulation of animals, and may have less non-specific tissue binding than an intact antibody (see, e.g. Wahl et al, 1983, J. Nucl. Med. 24:316).

An “Fv” fragment is the minimum fragment of an antibody that contains a complete target recognition and binding site. This region consists of a dimer of one heavy and one light chain variable domain in a tight, non-covalent association (VH-VL dimer). It is in this configuration that the three CDRs of each variable domain interact to define a target binding site on the surface of the VH-VL dimer. Often, the six CDRs confer target binding specificity to the antibody. However, in some instances even a single variable domain (or half of an Fv comprising only three CDRs specific for a target) can have the ability to recognize and bind target, although at a lower affinity than the entire binding site.

“Single-chain Fv” or “scFv” antibody binding fragments comprise the VH and VL domains of an antibody, where these domains are present in a single polypeptide chain. Generally, the Fv polypeptide further comprises a polypeptide linker between the VH and VL domains which enables the scFv to form the desired structure for target binding.

“Single domain antibodies” are composed of a single VH or VL domains which exhibit sufficient affinity to PD-1. In a specific embodiment, the single domain antibody is a camelized antibody {See, e.g., Riechmann, 1999, Journal of Immunological Methods 231:25-38).

In terms of structure, an antibody may have heavy (H) chains and light (L) chains interconnected by disulfide bonds. There are two types of light chain, lambda (λ) and kappa (κ). Each heavy and light chain contains a constant region and a variable region (or “domain”). Light and heavy chain variable regions contain a “framework” region interrupted by three hypervariable regions, also called “complementarity-determining regions” or “CDRs”. The extent of the framework region and CDRs have been defined (see, Kabat et al., Sequences of Proteins of Immunological Interest, and U.S. Department of Health and Human Services, 1991, which is hereby incorporated by reference). Preferably, the CDRs are defined according to Kabat method. The framework regions act to form a scaffold that provides, for positioning the CDRs in correct orientation by inter-chain, non-covalent interactions. The CDRs are primarily responsible for binding to an epitope of an antigen. The CDRs of each chain are typically referred to as “Complementarity Determining Region 1” or “CDR1”, “CDR2”, and “CDR3”, numbered sequentially starting from the N-terminus. The VL and VH domain of the antibody according to the invention may comprise four framework regions or “FR's”, which are referred to in the art and herein as “Framework region 1” or “FR1”, “FR2”, “FR3”, and “FR4”, respectively. These framework regions and complementary determining regions are preferably operably linked in the following order: FR1-CDR1-FR2-CDR2-FR3-CDR3-FR4 (from amino terminus to carboxy terminus). The term “antibody framework” as used herein refers to the part of the variable domain, either VL and/or VH, which serves as a scaffold for the antigen binding loops (CDRs) of this variable domain.

An “antibody heavy chain” as used herein, refers to the larger of the two types of polypeptide chains present in antibody conformations. The CDRs of the antibody heavy chain are typically referred to as “HCDR1”, “HCDR2” and “HCDR3”. The framework regions of the antibody heavy chain are typically referred to as “HFR1”, “HFR2”, “HFR3” and “HFR4”.

An “antibody light chain,” as used herein, refers to the smaller of the two types of polypeptide chains present in antibody conformations; κ and λ light chains refer to the two major antibody light chain isotypes. The CDRs of the antibody light chain are typically referred to as “LCDR1”, “LCDR2” and “LCDR3”. The framework regions of the antibody light chain are typically referred to as “LFR1”, “LFR2”, “LFR3” and “LFR4”.

With regard to the binding of an antibody to a target molecule, the terms “bind” or “binding” refer to peptides, polypeptides, proteins, fusion proteins, molecules and antibodies (including antibody fragments) that recognize and contact an antigen. Preferably, it refers to an antigen-antibody type interaction. The terms “specific binding”, “specifically binds to,” “specific for,” “selectively binds” and “selective for” a particular antigen (e.g., PD-1) or an epitope on a particular antigen (e.g., PD-1) mean that the antibody recognizes and binds a specific antigen, but does not substantially recognize or bind other molecules in a sample. For example, an antibody that specifically (or preferentially) binds to PD-1 or to a PD-1 epitope is an antibody that binds this PD-1 epitope for example with greater affinity, avidity, more readily, and/or with greater duration than it binds to other PD-1 epitopes or non-PD-1 epitopes. Preferably, the term “specific binding” means the contact between an antibody and an antigen with a binding affinity equal or lower than 10⁻⁷ M. In certain aspects, antibodies bind with affinities equal or lower than 10⁻⁸ M, 10⁻⁹ M or 10⁻¹° M.

As used herein “PD-1 antibody,” “anti-PD-1 antibody,” “PD-1 Ab,” “PD-1-specific antibody” or “anti-PD-1 Ab” are used interchangeably and refer to an antibody, as described herein, which specifically binds to PD-1, particularly human PD-1. In some embodiments, the antibody binds to the extracellular domain of PD-1. Particularly, an anti-PD-1 antibody is an antibody capable of binding to a PD-1 antigen and inhibits the PD-1-mediated signaling pathway, thereby enhancing immune responses such as T cell activation.

As used herein, the term “bifunctional molecule”, “bifunctional compound”, “bifunctional protein”, “Bicki”, “Bicki antibody”, “bifunctional antibody” and “bifunctional checkpoint inhibitors molecule” have the same meanings and can be interchangeably used. These terms refer to an antibody that recognizes one antigen by virtue of possessing at least one region (e.g. derived from a variable region of an antibody) that is specific for this antigen, and at least a second region that is a polypeptide. More specifically, the bifunctional molecule is a fusion protein of an antibody or a portion thereof, preferably an antigen binding fragment thereof with another polypeptide or polypeptide fragment thereof.

The term “chimeric antibody” as used herein, means an antibody or antigen-binding fragment, having a portion of heavy and/or light chain derived from one species, and the rest of the heavy and/or light chain derived from a different species. In an illustrative example, a chimeric antibody may comprise a constant region derived from human and a variable region from a non-human species, such as from a mouse.

As used herein, the term “humanized antibody” is intended to refer to antibodies in which CDR sequences derived from the germline of another mammalian species, such as a mouse, have been grafted onto human framework sequences (e.g. chimeric antibodies that contain minimal sequence derived from a non-human antibody). A “humanized antibody”, e.g., a non-human antibody, also refers to an antibody that has undergone humanization. A humanized antibody is generally a human immunoglobulin (recipient antibody) in which residues from one or more CDRs are replaced by residues from at least one CDR of a non-human antibody (donor antibody) while maintaining the desired specificity, affinity, and capacity of the original antibody. The donor antibody can be any suitable non-human antibody, such as a mouse, rat, rabbit, chicken, or non-human primate antibody having a desired specificity, affinity, or biological effect. In some instances, selected framework region residues of the recipient antibody are replaced by framework region residues from the donor antibody. Alternatively, selected framework region residues of the donor antibody are replaced by framework region residues from a human or humanized antibody. Additional framework region modifications may be made within the human framework sequences. Humanized antibodies thus may also comprise residues that are not found in either the recipient antibody or the donor antibody. Such amino acid modifications may be made to further refine antibody function and/or increased the humanization process. By “amino acid change” or “amino acid modification” is meant herein a change in the amino acid sequence of a polypeptide. “Amino acid modifications” include substitution, insertion and/or deletion in a polypeptide sequence. By “amino acid substitution” or “substitution” herein is meant the replacement of an amino acid at a particular position in a parent polypeptide sequence with another amino acid. By “amino acid insertion” or “insertion” is meant the addition of an amino acid at a particular position in a parent polypeptide sequence. By “amino acid deletion” or “deletion” is meant the removal of an amino acid at a particular position in a parent polypeptide sequence. The amino acid substitutions may be conservative. A conservative substitution is the replacement of a given amino acid residue by another residue having a side chain (“R-group”) with similar chemical properties (e.g., charge, bulk and/or hydrophobicity). As used herein, “amino acid position” or “amino acid position number” are used interchangeably and refer to the position of a particular amino acid in an amino acids sequence, generally specified with the one letter codes for the amino acids. The first amino acid in the amino acids sequence (i.e. starting from the N terminus) should be considered as having position 1.

A conservative substitution is the replacement of a given amino acid residue by another residue having a side chain (“R-group”) with similar chemical properties (e.g., charge, bulk and/or hydrophobicity). In general, a conservative amino acid substitution will not substantially change the functional properties of a protein. Conservative substitutions and the corresponding rules are well-described in the state of the art. For instance, conservative substitutions can be defined by substitutions within the groups of amino acids reflected in the following tables:

TABLE A
Amino Acid Residue

| Amino Acid groups | Amino Acid Residues |
|---|---|
| Acidic Residues | ASP and GLU |
| Basic Residues | LYS, ARG, and HIS |
| Hydrophilic Uncharged Residues | SER, THR, ASN, and GLN |
| Aliphatic Uncharged Residues | GLY, ALA, VAL, LEU, and ILE |
| Non-polar Uncharged Residues | CYS, MET, and PRO |
| Aromatic Residues | PHE, TYR, and TRP |

TABLE B
Alternative Conservative
Amino Acid Residue
Substitution Groups

| | | | |
|---|---|---|---|
| 1 | Alanine (A) | Serine (S) | Threonine (T) |
| 2 | Aspartic acid (D) | Glutamic acid (E) | |
| 3 | Asparagine (N) | Glutamine (Q) | |
| 4 | Arginine (R) | Lysine (K) | |
| 5 | Isoleucine (I) | Leucine (L) | Methionine (M) |
| 6 | Phenylalanine (F) | Tyrosine (Y) | Tryptophan (W) |

TABLE C
Further Alternative Physical
and Functional Classifications
of Amino Acid Residues

| | |
|---|---|
| Alcohol group- | S and T |
| containing residues | |
| Aliphatic residues | I, L, V, and M |
| Cycloalkenyl- | F, H, W, and Y |
| associated residues | |
| Hydrophobic residues | A, C, F, G, H, I, L, |
| | M, R, T, V, W, and Y |
| Negatively charged | D and E |
| residues | |
| Polar residues | C, D, E, H, K, N, Q, R, S, and T |
| Small residues | A, C, D, G, N, P, S, T, and V |
| Very small residues | A, G, and S |
| Residues involved in | A, C, D, E, G, H, K, |
| turn formation | N, Q, R, S, P, and T |
| Flexible residues | E, Q, T, K, S, G, P, D, E, and R |

As used herein, an “isolated antibody” is an antibody that has been separated and/or recovered from a component of its natural environment. An isolated antibody includes an antibody in situ within recombinant cells, since at least one component of the antibody's natural environment is not present. In some embodiments, an antibody is purified to homogeneity and/or to greater than 90%, 95% or 99% purity as determined by, for example, electrophoretic (e.g., SDS-PAGE, isoelectric focusing (IEF), capillary electrophoresis) or chromatographic (e.g., ion exchange or reverse phase HPLC) under reducing or non-reducing conditions.

The terms “derive from” and “derived from” as used herein refers to a compound having a structure derived from the structure of a parent compound or protein and whose structure is sufficiently similar to those disclosed herein and based upon that similarity, would be expected by one skilled in the art to exhibit the same or similar properties, activities and utilities as the claimed compounds. For example, a humanized antibody derived from a murine antibody refers to an antibody or antibody fragment that shares similar properties with the murine antibody, e.g. recognizes the same epitope, shares similar VH and VL with modified residues that participate and/or increased the humanization of the antibody.

The term “treatment” refers to any act intended to ameliorate the health status of patients such as therapy, prevention, prophylaxis and retardation of the disease or of the symptoms of the disease. It designates both a curative treatment and/or a prophylactic treatment of a disease. A curative treatment is defined as a treatment resulting in cure or a treatment alleviating, improving and/or eliminating, reducing and/or stabilizing a disease or the symptoms of a disease or the suffering that it causes directly or indirectly. A prophylactic treatment comprises both a treatment resulting in the prevention of a disease and a treatment reducing and/or delaying the progression and/or the incidence of a disease or the risk of its occurrence. In certain embodiments, such a term refers to the improvement or eradication of a disease, a disorder, an infection or symptoms associated with it. In other embodiments, this term refers to minimizing the spread or the worsening of cancers. Treatments according to the present invention do not necessarily imply 100% or complete treatment. Rather, there are varying degrees of treatment of which one of ordinary skill in the art recognizes as having a potential benefit or therapeutic effect. Preferably, the term “treatment” refers to the application or administration of a composition including one or more active agents to a subject, who has a disorder/disease, for instance associated with the signaling pathway mediated by PD-1.

As used herein, the terms “disorder” or “disease” refer to the incorrectly functioning organ, part, structure, or system of the body resulting from the effect of genetic or developmental errors, infection, poisons, nutritional deficiency or imbalance, toxicity, or unfavorable environmental factors. Preferably, these terms refer to a health disorder or disease e.g. an illness that disrupts normal physical or mental functions. More preferably, the term disorder refers to immune and/or inflammatory diseases that affect animals and/or humans, such as cancer.

The term “immune disease”, as used herein, refers to a condition in a subject characterized by cellular, tissue and/or organ injury caused by an immunologic reaction of the subject to its own cells, tissues and/or organs. The term “inflammatory disease” refers to a condition in a subject characterized by inflammation, e.g., chronic inflammation. Autoimmune disorders may or may not be associated with inflammation. Moreover, inflammation may or may not be caused by an autoimmune disorder.

The term “cancer” as used herein is defined as disease characterized by the rapid and uncontrolled growth of aberrant cells. Cancer cells can spread locally or through the bloodstream and lymphatic system to other parts of the body.

As used herein, the term “disease associated with or related to PD-1”, “PD-1 positive cancer” or “PD-1 positive infectious disease” is intended to refer to the cancer or infectious disease (e.g. caused by a virus and/or bacteria) which is resulted from PD-1 expression or has the symptom/characteristic of PD-1 expression, i.e. any condition that is caused by, exacerbated by, or otherwise linked to increased or decreased expression or activities of PD-1.

As used herein, the term “subject”, “host”, “individual,” or “patient” refers to human, including adult and child.

As used herein, a “pharmaceutical composition” refers to a preparation of one or more of the active agents, such as comprising a bifunctional molecule according to the invention, with optional other chemical components such as physiologically suitable carriers and excipients. The purpose of a pharmaceutical composition is to facilitate administration of the active agent to an organism. Compositions of the present invention can be in a form suitable for any conventional route of administration or use. In one embodiment, a “composition” typically intends a combination of the active agent, e.g., compound or composition, and a naturally-occurring or non-naturally-occurring carrier, inert (for example, a detectable agent or label) or active, such as an adjuvant, diluent, binder, stabilizer, buffers, salts, lipophilic solvents, preservative, adjuvant or the like and include pharmaceutically acceptable carriers. An “acceptable vehicle” or “acceptable carrier” as referred to herein, is any known compound or combination of compounds that are known to those skilled in the art to be useful in formulating pharmaceutical compositions.

“An effective amount” or a “therapeutic effective amount” as used herein refers to the amount of active agent required to confer therapeutic effect on the subject, either alone or in combination with one or more other active agents, e.g. the amount of active agent that is needed to treat the targeted disease or disorder, or to produce the desired effect. The “effective amount” will vary depending on the agent(s), the disease and its severity, the characteristics of the subject to be treated including age, physical condition, size, gender and weight, the duration of the treatment, the nature of concurrent therapy (if any), the specific route of administration and like factors within the knowledge and expertise of the health practitioner. These factors are well known to those of ordinary skill in the art and can be addressed with no more than routine experimentation. It is generally preferred that a maximum dose of the individual components or combinations thereof be used, that is, the highest safe dose according to sound medical judgment.

As used herein, the term “medicament” refers to any substance or composition with curative or preventive properties against disorders or diseases.

The term “in combination” as used herein refers to the use of more than one therapy (e.g., prophylactic and/or therapeutic agents). The use of the term “in combination” does not restrict the order in which therapies (e.g., prophylactic and/or therapeutic agents) are administered to a subject with a disease or disorder.

The terms “polynucleotide”, “nucleic acid” and “nucleic acid sequence” are equivalent and refer to a polymeric form of nucleotide of any length, for example RNA or DNA or analogs thereof. Nucleic acids (e.g., components, or portions, of the nucleic acids) of the present invention may be naturally occurring, modified or engineered, isolated and/or non-natural. Engineered nucleic acids include recombinant nucleic acids and synthetic nucleic acids. “Isolated nucleic acid encoding an anti-PD1 antibody” refers to one or more nucleic acid molecules encoding antibody heavy and light chains (or fragments thereof), including such nucleic acid molecule(s) in a single vector or separate vectors, and such nucleic acid molecule(s) present at one or more locations in a host cell. As used herein, the terms “nucleic acid construct”, “plasmid”, and “vector” are equivalent and refer to a nucleic acid molecule that serves to transfer a passenger nucleic acid sequence, such as DNA or RNA, into a host cell.

As used herein, the term “host cell” is intended to include any individual cell or cell culture that can be or has been recipient of vectors, exogenous nucleic acid molecules, and polynucleotides encoding the antibody construct of the present invention; and/or recipients of the antibody construct itself. The introduction of the respective material into the cell can be carried out by way of transformation, transfection and the like. The term “host cell” is also intended to include progeny or potential progeny of a single cell. Host cells include for example bacterial, microbial, plant and animal cells.

“Immune cells” as used herein refers to cells involved in innate and adaptive immunity for example such as white blood cells (leukocytes) which are derived from hematopoietic stem cells (HSC) produced in the bone marrow, lymphocytes (T cells, B cells, natural killer (NK) cells and Natural Killer T cells (NKT) and myeloid-derived cells (neutrophil, eosinophil, basophil, monocyte, macrophage, dendritic cells). In particular, the immune cell can be selected in the non-exhaustive list comprising B cells, T cells, in particular CD4+ T cells and CD8+ T cells, NK cells, NKT cells, APC cells, dendritic cells and monocytes. “T cell” as used herein includes for example CD4+ T cells, CD8+ T cells, T helper 1 type T cells, T helper 2 type T cells, T helper 17 type T cells and inhibitory T cells.

As used herein, the term “T effector cell”, “T eff” or “effector cell” describes a group of immune cells that includes several T cell types that actively respond to a stimulus, such as co-stimulation. It particularly includes T cells which function to eliminate antigen (e.g., by producing cytokines which modulate the activation of other cells or by cytotoxic activity). It notably includes CD4+, CD8+, Treg cells, cytotoxic T cells and helper T cells (Th1 and Th2).

As used herein, the term “regulatory T cell”, Treg cells” or “T reg” refers to a subpopulation of T cells that modulate the immune system, maintain tolerance to self-antigens, and prevent autoimmune disease. Tregs are immunosuppressive and generally suppress or downregulate induction and proliferation of effector T cells. Tregs express the biomarkers CD4, FOXP3, and CD25 and are thought to be derived from the same lineage as naïve CD4 cells.

The term “exhausted T cell” refers to a population of T cell in a state of dysfunction (i.e. “exhaustion”). T cell exhaustion is characterized by progressive loss of function, changes in transcriptional profiles and sustained expression of inhibitory receptors. Exhausted T cells lose their cytokines production capacity, their high proliferative capacity and their cytotoxic potential, which eventually leads to their deletion. Exhausted T cells typically indicate higher levels of CD43, CD69 and inhibitory receptors combined with lower expression of CD62L and CD127.

The term “immune response” refers to the action of, for example, lymphocytes, antigen presenting cells, phagocytic cells, granulocytes, and soluble macromolecules produced by the above cells or the liver (including antibodies, cytokines, and complements) that results in selective damage to, destruction of, or elimination from the human body of invading pathogens, cells or tissues infected with pathogens, cancerous cells, or, in cases of autoimmunity or pathological inflammation, normal human cells or tissues.

The term “antagonist” as used herein, refers to a substance that block or reduces the activity or functionality of another substance. Particularly, this term refers to an antibody that binds to a cellular receptor (e.g. PD-1) as a reference substance (e.g. PD-L1 and/or PD-L2), preventing it from producing all or part of its usual biological effects (e.g. the creation of an immune suppressive microenvironment). The antagonist activity of an antibody according to the invention may be assessed by competitive ELISA.

As used herein, the term “isolated” indicates that the recited material (e.g., antibody, polypeptide, nucleic acid, etc.) is substantially separated from, or enriched relative to, other materials with which it occurs in nature. Particularly, an “isolated” antibody is one which has been identified and separated and/or recovered from a component of its natural environment. For example, the isolated antibody is purified (1) to greater than 75% by weight of antibody as determined by the Lowry method, or (2) to homogeneity by SDS-PAGE under reducing or non-reducing conditions. Isolated antibody includes the antibody in situ within recombinant cells since at least one component of the antibody's natural environment will not be present. Ordinarily, however, isolated antibody will be prepared by at least one purification step.

The term “and/or” as used herein is to be taken as specific disclosure of each of the two specified features or components with or without the other. For example, “A and/or B” is to be taken as specific disclosure of each of (i) A, (ii) B and (iii) A and B, just as if each is set out individually.

The term “a” or “an” can refer to one of or a plurality of the elements it modifies (e.g., “a reagent” can mean one or more reagents) unless it is contextually clear either one of the elements or more than one of the elements is described.

The term “about” as used herein in connection with any and all values (including lower and upper ends of numerical ranges) means any value having an acceptable range of deviation of up to +/−10% (e.g., +/−0.5%, +/−1%, +/−1.5%, +/−2%, +/−2.5%, +/−3%, +/−3.5%, +/−4%, +/−4.5%, +/−5%, +/−5.5%, +/−6%, +/−6.5%, +/−7%, +/−7.5%, +/−8%, +/−8.5%, +/−9%, +/−9.5%). The use of the term “about” at the beginning of a string of values modifies each of the values (i.e. “about 1, 2 and 3” refers to about 1, about 2 and about 3). Further, when a listing of values is described herein (e.g. about 50%, 60%, 70%, 80%, 85% or 86%) the listing includes all intermediate and fractional values thereof (e.g., 54%, 85.4%).

### Anti-PD-1 Antibody

The bifunctional molecule according to the invention comprises a first entity that comprises an anti-hPD-1 antibody or an antigen binding fragment thereof.

Provided herein are antibodies that particularly bind to human PD-1. In some aspects, the antibody specifically binds to human PD-1, preferably to the extracellular domain of human PD-1. In some aspects, the antibody selectively binds to one or more of full-length human PD-1, PD-1Aex2, PD-1Aex3, PD-1Aex2,3 and PD-1Aex2,3,4.

In some aspects, the anti-PD1 antibody is an isolated antibody, particularly a non-natural isolated antibody. Such isolated anti-PD1 antibody can be prepared by at least one purification step. In some embodiments, an isolated anti-PD1 antibody is purified to at least 80%, 85%, 90%, 95% or 99% by weight. In some embodiments, an isolated anti-PD1 isolated antibody is provided as a solution comprising at least 85%, 90%, 95%, 98%, 99% to 100% by weight of an antibody, the remainder of the weight comprising the weight of other solutes dissolved in the solvent.

Preferably, such antibody has the ability to block or inhibit the interaction between PD-1 and at least one of its ligand (e.g. PD-L1 and/or PD-L2). The ability to “block binding” or “block interaction” or “inhibit interaction” as used herein refers to the ability of an antibody or antigen-binding fragment to prevent the binding interaction between two molecules (e.g. PD-1 and its ligand PD-L1 and/or PD-L2) to any detectable degree.

Preferably, the anti-PD1 antibody or antigen binding fragment thereof is an antagonist of the binding of human PD-L1 and/or PD-L2 to human PD-1, more preferably of human PD-L1 and PD-L2 to human PD-1.

In certain embodiments, the anti-hPD1 antibody or antigen-binding fragment inhibits the binding interaction between PD-1 and at least one of its ligands (e.g. PD-L1 and/or PD-L2, preferably PD-L1 and PD-L2) by at least 50%. In certain embodiments, this inhibition may be greater than 60%, greater than 70%, greater than 80%, or greater than 90%.

Anti-hPD1 antibodies according to this invention may comprise immunoglobulins, immunoglobulin of any class, such as IgD, IgE, IgG, IgA, or IgM (or sub-class thereof), immunoglobulin chains or fragments thereof (such as Fv, Fab, Fab′, F(ab′)2, scFv or other antigen-binding subsequences of antibodies) which contain minimal sequence derived from a non-human (e.g. murine) immunoglobulin targeting human PD-1. Preferably, the anti-hPD-1 antibody according to the invention derives from IgG1, IgG2, IgG3 or IgG4, preferably from an IgG4 or an IgG1.

In one embodiment, the antigen-binding fragment of an antibody comprises a heavy chain comprising a heavy chain variable domain comprising HCDR1, HCDR2 and HCDR3 and a light chain comprising a variable domain comprising LCDR1, LCDR2 and LCDR3, and a fragment of a heavy chain constant domain. By a fragment of a heavy chain constant domain, it should be understood that the antigen-binding fragment therefore comprises at least a portion of a full heavy chain constant domain. As examples, a heavy chain constant domain may comprise or consist of at least the C_{H}1 domain of a heavy chain, or at least the C_{H}1 and the C_{H}2 domains of a heavy chain, or at least the C_{H}1, C_{H}2 and C_{H}3 domains of a heavy chain. A fragment of a heavy chain constant domain may also be defined as comprising at least a portion of the Fc domain of the heavy chain. Accordingly, antigen-binding fragment of an antibody encompasses the Fab portion of a full antibody, the F(ab′)₂ portion of a full antibody, the Fab′ portion of a full antibody. The heavy chain constant domain may also comprise or consist in a full heavy chain constant domain, for example illustrated in the present description, wherein several full heavy chain constant domains are described. In a particular embodiment of the invention, and when the antigen-binding fragment of an antibody comprises a fragment of a heavy chain constant domain comprising or consisting in a portion of a full heavy chain constant domain, the heavy chain constant domain fragment may consist of at least 10 amino acid residues; or may consist of 10 to 300 amino acid residues, in particular 210 amino acid residues.

Preferably, the antibody against human PD-1 is a monoclonal antibody. The term “monoclonal antibody” as used herein refers to an antibody obtained from a population of substantially homogeneous antibodies, i.e., the individual antibodies comprising the population are identical and/or bind the same epitope. Preferably, such monoclonal antibodies (mAbs) are from a mammalian, such as mice, rodents, rabbit, goat, primates, non-human primates or humans. Techniques for preparing such monoclonal antibodies may be found in, e.g., Stites et al. (eds.) BASIC AND CLINICAL IMMUNOLOGY (4th ed.) Lange Medical Publications, Los Altos, Calif., and references cited therein; Harlow and Lane (1988) ANTIBODIES: A LABORATORY MANUAL CSH Press; Goding (1986) MONOCLONAL ANTIBODIES: PRINCIPLES AND PRACTICE (2d ed.) Academic Press, New York, N.Y.

In one embodiment, the anti-PD1 antibody can be selected from the group consisting of Pembrolizumab (Keytruda—MK-3475), Nivolumab (Opdivo, MDX-1106, BMS-936558, ONO-4538), Pidilizumab (CT-011), Cemiplimab (Libtayo) PDR001, monoclonal antibodies 5C4, 17D8, 2D3, 4H1, 4A11, 7D3, and 5F4, described in WO 2006/121168.

In some embodiments, the anti-hPD1 antibody provided herein is an isolated antibody.

In certain embodiments, the anti-hPD1 antibody provided herein is a chimeric antibody. In one example, the chimeric antibody comprises a non-human variable region (e.g., a variable region derived from a mouse, rat, hamster, rabbit, or non-human primate, such as a monkey) and a human constant region. In a further example, a chimeric antibody is a “class switched” antibody in which the class or subclass has been changed from that of the parent antibody. Chimeric antibodies include antigen-binding fragments thereof.

In certain embodiments, the anti-hPD1 antibody is a humanized antibody. A humanized antibody typically comprises one or more variable domains in which CDRs (or portions thereof) are derived from a non-human antibody, and FRs (or portions thereof) are derived from human or humanized antibody sequences. Alternatively, some FR residues can be substituted to restore or improve antibody specificity, affinity and/or humanization. A humanized antibody optionally will also comprise at least a portion of a human or humanized constant region (Fc). Methods of antibodies humanization are well known in the art see for example, Winter and Milstein, Nature, 1991, 349:293-299; Riechmann et al., Nature, 332, pp. 323 (1988); Verhoeyen et al., Science, 239, pp. 1534 (1988), Rader et al, Proc. Nat. Acad. Sci. U.S.A., 1998, 95:8910-8915; Steinberger et al, J. Biol. Chem., 2000, 275:36073-36078; Queen et al, Proc. Natl. Acad. Sci. U.S.A., 1989, 86: 10029-10033; Almagro, J. C. and Fransson, J., Front. Biosci. 13 (2008) 1619-1633; Kashmiri, S. V. et al, Methods 36 (2005) 25-34 (describing SDR (a-CDR) grafting); Padlan, E. A., Mol. Immunol. 28 (1991) 489-498 (describing “resurfacing”); Dall'Acqua, W. F. et al, Methods 36 (2005) 43-60 (describing “FR shuffling”); and Osbourn, J. et al, Methods 36 (2005) 61-68 and Klimka, A. et al, Br. J. Cancer 83 (2000) 252-260 (describing the “guided selection” approach to FR shuffling) and U.S. Pat. Nos. 5,585,089, 5,693,761, 5,693,762, 5,821,337, 7,527,791, 6,982,321, and 7,087,409; and 6,180,370. Preferably, the humanized antibody against human PD-1 is a monoclonal antibody.

Particularly, a humanized antibody is one that has a T20 humanness score of at least 80% or at least 85%, more preferably at least 88%, even more preferably at least 90%, most preferably a T20 humanness score comprised between 85% and 95%, preferably between 88% and 92%.

“Humanness” is generally measured using the T20 score analyzer to quantify the humanness of the variable region of monoclonal antibodies as described in Gao S H, Huang K, Tu H, Adler A S. BMC Biotechnology. 2013: 13:55. T20 humanness score is a parameter commonly used in the field of antibody humanization first disclosed by Gao et al (BMC Biotechnol., 2013, 13, 55). T20 humanness score is usually used in patent application for defining a humanized antibody (e.g., WO15161311, WO17127664, WO18136626, WO18190719, WO19060750, or WO19170677).

A web-based tool is provided to calculate the T20 score of antibody sequences using the T20 Cutoff Human Databases: http://abAnalyzer.lakepharma.com. In computing a T20 score, an input VH, VK, or VL variable region protein sequence is first assigned Kabat numbering, and CDR residues are identified. The full-length sequence or the framework only sequence (with CDR residues removed) is compared to every sequence in a respective antibody database using the blastp protein-protein BLAST algorithm. The sequence identity between each pairwise comparison is isolated, and after every sequence in the database has been analyzed, the sequences are sorted from high to low based on the sequence identity to the input sequence. The percent identity of the Top 20 matched sequences is averaged to obtain the T20 score.

For each chain type (VH, VK, VL) and sequence length (full-length or framework only) in the “All Human Databases,” each antibody sequence was scored with its respective database using the T20 score analyzer. The T20 score was obtained for the top 20 matched sequences after the input sequence itself was excluded (the percent identity of sequences 2 through 21 were averaged since sequence 1 was always the input antibody itself). The T20 scores for each group were sorted from high to low. The decrease in score was roughly linear for most of the sequences; however, the T20 scores for the bottom ^{˜}15% of antibodies started decreasing sharply. Therefore, the bottom 15 percent of sequences were removed and the remaining sequences formed the T20 Cutoff Human Databases, where the T20 score cutoff indicates the lowest T20 score of a sequence in the new database.

Accordingly, the humanized anti-PD1 antibody comprised in the bifunctional molecule according to the invention has a T20 humanness score of at least 80% or at least 85%, more preferably at least 88%, even more preferably at least 90%, most preferably a T20 humanness score comprised between 85% and 95%, preferably between 88% and 92%.

In one embodiment, the anti-PD1 antibody can be selected from the group consisting of Pembrolizumab (also known as Keytruda lambrolizumab, MK-3475), Nivolumab (Opdivo, MDX-1106, BMS-936558, ONO-4538), Pidilizumab (CT-011), Cemiplimab (Libtayo), Camrelizumab, AUNP12, AMP-224, AGEN-2034, BGB-A317 (Tisleizumab), PDR001 (spartalizumab), MK-3477, SCH-900475, PF-06801591, JNJ-63723283, genolimzumab (CBT-501), LZM-009, BCD-100, SHR-1201, BAT-1306, AK-103 (HX-008), MEDI-0680 (also known as AMP-514) MEDI0608, JS001 (see Si-Yang Liu et al., J. Hematol. Oncol. 10:136 (2017)), B1-754091, CBT-501, INCSHR1210 (also known as SHR-1210), TSR-042 (also known as ANB011), GLS-010 (also known as WBP3055), AM-0001 (Armo), STI-1110 (see WO 2014/194302), AGEN2034 (see WO 2017/040790), MGA012 (see WO 2017/19846), or 1B1308 (see WO 2017/024465, WO 2017/025016, WO 2017/132825, and WO 2017/133540), monoclonal antibodies 5C4, 17D8, 2D3, 4H1, 4A11, 7D3, and 5F4, described in WO 2006/121168. Bifunctional or bispecific molecules targeting PD-1 are also known such as RG7769 (Roche), XmAb20717 (Xencor), MED15752 (AstraZeneca), FS118 (F-star), SL-279252 (Takeda) and XmAb23104 (Xencor).

In a particular embodiment, the anti-PD1 antibody can be Pembrolizumab (also known as Keytruda lambrolizumab, MK-3475) or Nivolumab (Opdivo, MDX-1106, BMS-936558, ONO-4538).

A particular example of a humanized anti-hPD1 antibody is described hereafter by its CDRs, framework regions and Fc and hinge region.

### CDR

“Complementarity determining regions” or “CDRs” are known in the art as referring to non-contiguous sequences of amino acids within antibody variable regions, which confer antigen specificity and binding affinity. The precise amino acid sequence boundaries of a given CDR can be readily determined using any of a number of well-known schemes, including those described by Kabat et al., (Sequences of Proteins of Immunological Interest 5th ed. (1991) “Kabat” numbering scheme); Al-Lazikani et al., 1997, J. Mol. Biol, 273:927-948 (“Chothia” numbering scheme); MacCallum et al, 1996, J. Mol. Biol. 262:732-745 (“Contact” numbering scheme); Lefranc et al., Dev. Comp. Immunol., 2003, 27:55-77 (“IMGT” numbering scheme); and Honegge and Pluckthun, J. Mol. Biol, 2001, 309:657-70 (“AHo” numbering scheme). Unless otherwise specified, the numbering scheme used for identification of a particular CDR herein is the Kabat numbering scheme.

In one embodiment, the bifunctional molecule comprises a humanized anti-hPD-1 antibody or an antigen binding fragment thereof. The CDRs regions of the humanized antibody may be derived from a murine antibody and have been optimized to i) provide a safe humanized antibody with a very high level of humanization (superior to 85%) and stability; and ii) increase the antibody properties, more particularly a higher manufacturability and a higher production yield when produced in mammalian cells such as COS and HCO cells, while preserving an antagonist activity (i.e. inhibition of the binding of human PD-L1 to human PD-1), as they have a binding affinity (KD) for a human PD-1 less than 10⁻⁷ M, preferably less than 10⁻⁸ M.

In a very particular embodiment, the bifunctional molecule comprises an anti-human-PD-1 antibody or antigen binding fragment thereof that comprises:

(i) a heavy chain variable domain comprising HCDR1, HCDR2 and HCDR3, and
(ii) a light chain variable domain comprising LCDR1, LCDR2 and LCDR3,
wherein:
the heavy chain CDR1 (HCDR1) comprises or consists of an amino acid sequence of SEQ ID NO: 1, optionally with one, two or three modification(s) selected from substitution(s), addition(s), deletion(s) and any combination thereof;
the heavy chain CDR2 (HCDR2) comprises or consists of an amino acid sequence of SEQ ID NO: 2, optionally with one, two or three modification(s) selected from substitution(s), addition(s), deletion(s) and any combination thereof;
the heavy chain CDR3 (HCDR3) comprises or consists of an amino acid sequence of SEQ ID NO: 3 wherein X1 is D or E and X2 is selected from the group consisting of T, H, A, Y, N, E and S, preferably in the group consisting of H, A, Y, N, E; optionally with one, two or three modification(s) selected from substitution(s), addition(s), deletion(s) and any combination thereof at any position but positions 2, 3, 7 and 8 of SEQ ID NO: 3;
the light chain CDR1 (LCDR1) comprises or consists of an amino acid sequence of SEQ ID NO: 12 wherein X is G or T, optionally with one, two or three modification(s) selected from substitution(s), addition(s), deletion(s) and any combination thereof at any position but positions 10, 11 and 16 of SEQ ID NO: 12;
the light chain CDR2 (LCDR2) comprises or consists of an amino acid sequence of SEQ ID NO: 15, optionally with one, two or three modification(s) selected from substitution(s), addition(s), deletion(s) and any combination thereof; and
the light chain CDR3 (LCDR3) comprises or consists of an amino acid sequence of SEQ ID NO:16, optionally with one, two or three modification(s) selected from substitution(s), addition(s), deletion(s) and any combination thereof at any position but positions 1 and 6 of SEQ ID NO: 16.

In another embodiment, the bifunctional molecule comprises a humanized anti-hPD-1 antibody or an antigen binding fragment thereof that comprises the HCDR1, HCDR2, LCDR2 and LCDR3 as specified above, the heavy chain CDR3 (HCDR3) comprising or consisting of an amino acid sequence of SEQ ID NO: 3 wherein either X1 is D and X2 is selected from the group consisting of T, H, A, Y, N, E, and S preferably in the group consisting of H, A, Y, N, E; or X1 is E and X2 is selected from the group consisting of T, H, A, Y, N, E and S, preferably in the group consisting of H, A, Y, N, E and S; optionally with one, two or three modification(s) selected from substitution(s), addition(s), deletion(s) and any combination thereof at any position but positions 2, 3, 7 and 8 of SEQ ID NO: 3; and the light chain CDR1 (LCDR1) comprising or consisting of an amino acid sequence of SEQ ID NO: 12 wherein X is G or T, optionally with one, two or three modification(s) selected from substitution(s), addition(s), deletion(s) and any combination thereof at any position but positions 5, 6, 10, 11 and 16 of SEQ ID NO: 12.

In another embodiment, the bifunctional molecule comprises a humanized anti-hPD-1 antibody or an antigen binding fragment thereof that comprises the HCDR1, HCDR2, LCDR2 and LCDR3 as specified above, and
the heavy chain CDR3 (HCDR3) which comprises or consists of an amino acid sequence of SEQ ID NO: 4, 5, 6, 7, 8, 9, 10 or 11 optionally with one, two or three modification(s) selected from substitution(s), addition(s), deletion(s) and any combination thereof at any position but positions 2, 3, 7 and 8 of SEQ ID NO: 4, 5, 6, 7, 8, 9, 10 or 11; and
the light chain CDR1 (LCDR1) which comprises or consists of an amino acid sequence of SEQ ID NO: 13 or SEQ ID NO:14, optionally with one, two or three modification(s) selected from substitution(s), addition(s), deletion(s) and any combination thereof at any position but positions 5, 6, 10, 11 and 16 of SEQ ID NO: 13 or SEQ ID NO:14.

In another embodiment, the bifunctional molecule comprises a humanized anti-hPD-1 antibody or an antigen binding fragment thereof that comprises the HCDR1, HCDR2, LCDR2 and LCDR3 as specified above, and
the heavy chain CDR3 (HCDR3) which comprises or consists of an amino acid sequence of SEQ ID NO: 4, optionally with one, two or three modification(s) selected from substitution(s), addition(s), deletion(s) and any combination thereof at any position but positions 2, 3, 7 and 8 of SEQ ID NO: 4; and the light chain CDR1 (LCDR1) which comprises or consists of an amino acid sequence of SEQ ID NO: 13, optionally with one, two or three modification(s) selected from substitution(s), addition(s), deletion(s) and any combination thereof at any position but positions 5, 6, 10, 11 and 16 of SEQ ID NO: 13; or
the heavy chain CDR3 (HCDR3) which comprises or consists of an amino acid sequence of SEQ ID NO: 5, optionally with one, two or three modification(s) selected from substitution(s), addition(s), deletion(s) and any combination thereof at any position but positions 2, 3, 7 and 8 of SEQ ID NO: 5; and the light chain CDR1 (LCDR1) which comprises or consists of an amino acid sequence of SEQ ID NO: 13, optionally with one, two or three modification(s) selected from substitution(s), addition(s), deletion(s) and any combination thereof at any position but positions 5, 6, 10, 11 and 16 of SEQ ID NO: 13; or
the heavy chain CDR3 (HCDR3) which comprises or consists of an amino acid sequence of SEQ ID NO: 6, optionally with one, two or three modification(s) selected from substitution(s), addition(s), deletion(s) and any combination thereof at any position but positions 2, 3, 7 and 8 of SEQ ID NO: 6; and the light chain CDR1 (LCDR1) which comprises or consists of an amino acid sequence of SEQ ID NO: 13, optionally with one, two or three modification(s) selected from substitution(s), addition(s), deletion(s) and any combination thereof at any position but positions 5, 6, 10, 11 and 16 of SEQ ID NO: 13; or
the heavy chain CDR3 (HCDR3) which comprises or consists of an amino acid sequence of SEQ ID NO: 7, optionally with one, two or three modification(s) selected from substitution(s), addition(s), deletion(s) and any combination thereof at any position but positions 2, 3, 7 and 8 of SEQ ID NO:7; and the light chain CDR1 (LCDR1) which comprises or consists of an amino acid sequence of SEQ ID NO: 13, optionally with one, two or three modification(s) selected from substitution(s), addition(s), deletion(s) and any combination thereof at any position but positions 5, 6, 10, 11 and 16 of SEQ ID NO: 13; or
the heavy chain CDR3 (HCDR3) which comprises or consists of an amino acid sequence of SEQ ID NO: 8 optionally with one, two or three modification(s) selected from substitution(s), addition(s), deletion(s) and any combination thereof at any position but positions 2, 3, 7 and 8 of SEQ ID NO: 8; and the light chain CDR1 (LCDR1) which comprises or consists of an amino acid sequence of SEQ ID NO: 13, optionally with one, two or three modification(s) selected from substitution(s), addition(s), deletion(s) and any combination thereof at any position but positions 5, 6, 10, 11 and 16 of SEQ ID NO: 13; or
the heavy chain CDR3 (HCDR3) which comprises or consists of an amino acid sequence of SEQ ID NO: 9 optionally with one, two or three modification(s) selected from substitution(s), addition(s), deletion(s) and any combination thereof at any position but positions 2, 3, 7 and 8 of SEQ ID NO: 9; and the light chain CDR1 (LCDR1) which comprises or consists of an amino acid sequence of SEQ ID NO: 13, optionally with one, two or three modification(s) selected from substitution(s), addition(s), deletion(s) and any combination thereof at any position but positions 5, 6, 10, 11 and 16 of SEQ ID NO: 13; or
the heavy chain CDR3 (HCDR3) which comprises or consists of an amino acid sequence of SEQ ID NO: 10 optionally with one, two or three modification(s) selected from substitution(s), addition(s), deletion(s) and any combination thereof at any position but positions 2, 3, 7 and 8 of SEQ ID NO: 10; and the light chain CDR1 (LCDR1) which comprises or consists of an amino acid sequence of SEQ ID NO: 13, optionally with one, two or three modification(s) selected from substitution(s), addition(s), deletion(s) and any combination thereof at any position but positions 5, 6, 10, 11 and 16 of SEQ ID NO: 13; or
the heavy chain CDR3 (HCDR3) which comprises or consists of an amino acid sequence of SEQ ID NO: 11 optionally with one, two or three modification(s) selected from substitution(s), addition(s), deletion(s) and any combination thereof at any position but positions 2, 3, 7 and 8 of SEQ ID NO: 11; and the light chain CDR1 (LCDR1) which comprises or consists of an amino acid sequence of SEQ ID NO: 13, optionally with one, two or three modification(s) selected from substitution(s), addition(s), deletion(s) and any combination thereof at any position but positions 5, 6, 10, 11 and 16 of SEQ ID NO: 13; or
the heavy chain CDR3 (HCDR3) which comprises or consists of an amino acid sequence of SEQ ID NO: 4, optionally with one, two or three modification(s) selected from substitution(s), addition(s), deletion(s) and any combination thereof at any position but positions 2, 3, 7 and 8 of SEQ ID NO: 4; and the light chain CDR1 (LCDR1) which comprises or consists of an amino acid sequence of SEQ ID NO: 14, optionally with one, two or three modification(s) selected from substitution(s), addition(s), deletion(s) and any combination thereof at any position but positions 5, 6, 10, 11 and 16 of SEQ ID NO: 14; or
the heavy chain CDR3 (HCDR3) which comprises or consists of an amino acid sequence of SEQ ID NO: 5, optionally with one, two or three modification(s) selected from substitution(s), addition(s), deletion(s) and any combination thereof at any position but positions 2, 3, 7 and 8 of SEQ ID NO: 5; and the light chain CDR1 (LCDR1) which comprises or consists of an amino acid sequence of SEQ ID NO: 14, optionally with one, two or three modification(s) selected from substitution(s), addition(s), deletion(s) and any combination thereof at any position but positions 5, 6, 10, 11 and 16 of SEQ ID NO: 14; or
the heavy chain CDR3 (HCDR3) which comprises or consists of an amino acid sequence of SEQ ID NO: 6, optionally with one, two or three modification(s) selected from substitution(s), addition(s), deletion(s) and any combination thereof at any position but positions 2, 3, 7 and 8 of SEQ ID NO: 6; and the light chain CDR1 (LCDR1) which comprises or consists of an amino acid sequence of SEQ ID NO: 14, optionally with one, two or three modification(s) selected from substitution(s), addition(s), deletion(s) and any combination thereof at any position but positions 5, 6, 10, 11 and 16 of SEQ ID NO: 14; or
the heavy chain CDR3 (HCDR3) which comprises or consists of an amino acid sequence of SEQ ID NO: 7, optionally with one, two or three modification(s) selected from substitution(s), addition(s), deletion(s) and any combination thereof at any position but positions 2, 3, 7 and 8 of SEQ ID NO:7; and the light chain CDR1 (LCDR1) which comprises or consists of an amino acid sequence of SEQ ID NO: 14, optionally with one, two or three modification(s) selected from substitution(s), addition(s), deletion(s) and any combination thereof at any position but positions 5, 6, 10, 11 and 16 of SEQ ID NO: 14; or
the heavy chain CDR3 (HCDR3) which comprises or consists of an amino acid sequence of SEQ ID NO: 8 optionally with one, two or three modification(s) selected from substitution(s), addition(s), deletion(s) and any combination thereof at any position but positions 2, 3, 7 and 8 of SEQ ID NO: 8; and the light chain CDR1 (LCDR1) which comprises or consists of an amino acid sequence of SEQ ID NO: 14, optionally with one, two or three modification(s) selected from substitution(s), addition(s), deletion(s) and any combination thereof at any position but positions 5, 6, 10, 11 and 16 of SEQ ID NO: 14; or
the heavy chain CDR3 (HCDR3) which comprises or consists of an amino acid sequence of SEQ ID NO: 9 optionally with one, two or three modification(s) selected from substitution(s), addition(s), deletion(s) and any combination thereof at any position but positions 2, 3, 7 and 8 of SEQ ID NO: 9; and the light chain CDR1 (LCDR1) which comprises or consists of an amino acid sequence of SEQ ID NO: 14, optionally with one, two or three modification(s) selected from substitution(s), addition(s), deletion(s) and any combination thereof at any position but positions 5, 6, 10, 11 and 16 of SEQ ID NO: 14; or
the heavy chain CDR3 (HCDR3) which comprises or consists of an amino acid sequence of SEQ ID NO: 10 optionally with one, two or three modification(s) selected from substitution(s), addition(s), deletion(s) and any combination thereof at any position but positions 2, 3, 7 and 8 of SEQ ID NO: 10; and the light chain CDR1 (LCDR1) which comprises or consists of an amino acid sequence of SEQ ID NO: 14, optionally with one, two or three modification(s) selected from substitution(s), addition(s), deletion(s) and any combination thereof at any position but positions 5, 6, 10, 11 and 16 of SEQ ID NO: 14; or
the heavy chain CDR3 (HCDR3) which comprises or consists of an amino acid sequence of SEQ ID NO: 11 optionally with one, two or three modification(s) selected from substitution(s), addition(s), deletion(s) and any combination thereof at any position but positions 2, 3, 7 and 8 of SEQ ID NO: 11; and the light chain CDR1 (LCDR1) which comprises or consists of an amino acid sequence of SEQ ID NO: 14, optionally with one, two or three modification(s) selected from substitution(s), addition(s), deletion(s) and any combination thereof at any position but positions 5, 6, 10, 11 and 16 of SEQ ID NO: 14.

In a particular aspect, the modifications are substitutions, in particular conservative substitutions.

In one embodiment, the anti-human-PD-1 antibody or antigen binding fragment thereof comprises (i) a heavy chain comprising a CDR1 of SEQ ID NO: 1, a CDR2 of SEQ ID NO: 2 and a CDR3 of SEQ ID NO: 3 wherein X1 is D or E and X2 is selected from the group consisting of T, H, A, Y, N, E and S, preferably in the group consisting of H, A, Y, N and E; and (ii) a light chain comprising a CDR1 of SEQ ID NO: 12 wherein X is G or T, a CDR2 of SEQ ID NO: 15 and a CDR3 of SEQ ID NO: 16.

In one embodiment, the anti-human-PD-1 antibody or antigen binding fragment thereof comprises (i) a heavy chain comprising a CDR1 of SEQ ID NO: 1, a CDR2 of SEQ ID NO: 2 and a CDR3 of SEQ ID NO: 3 wherein X1 is D and X2 is selected from the group consisting of T, H, A, Y, N and E, preferably in the group consisting of H, A, Y, N and E; or wherein X1 is E and X2 is selected from the group consisting of T, H, A, Y, N, E, and S, preferably in the group consisting of H, A, Y, N, E and 5; and (ii) a light chain comprising a CDR1 of SEQ ID NO: 12 wherein X is G or T, a CDR2 of SEQ ID NO: 15 and a CDR3 of SEQ ID NO: 16.

In one embodiment, the anti-human-PD-1 antibody or antigen binding fragment thereof comprises (i) a heavy chain comprising a CDR1 of SEQ ID NO: 1, a CDR2 of SEQ ID NO: 2 and a CDR3 of SEQ ID NO: 3 wherein X1 is D and X2 is selected from the group consisting of T, H, A, Y, N and E, preferably in the group consisting of H, A, Y, N and E; and (ii) a light chain comprising a CDR1 of SEQ ID NO: 12 wherein X is G or T, a CDR2 of SEQ ID NO: 15 and a CDR3 of SEQ ID NO: 16.

In one embodiment, the anti-human-PD-1 antibody or antigen binding fragment thereof comprises (i) a heavy chain comprising a CDR1 of SEQ ID NO: 1, a CDR2 of SEQ ID NO: 2 and a CDR3 of SEQ ID NO: 3 wherein X1 is E and X2 is selected from the group consisting of T, H, A, Y, N, E, and S, preferably in the group consisting of H, A, Y, N, E and S; and (ii) a light chain comprising a CDR1 of SEQ ID NO: 12 wherein X is G or T, a CDR2 of SEQ ID NO: 15 and a CDR3 of SEQ ID NO: 16.

In another embodiment, the anti-human-PD-1 antibody or antigen binding fragment thereof comprises or consists essentially of (i) a heavy chain comprising a CDR1 of SEQ ID NO: 1, a CDR2 of SEQ ID NO: 2 and a CDR3 of SEQ ID NO: 4, 5, 6, 7, 8, 9, 10 or 11; and (ii) a light chain comprising a CDR1 of SEQ ID NO: 13 or SEQ ID NO:14, a CDR2 of SEQ ID NO: 15 and a CDR3 of SEQ ID NO: 16.

In another embodiment, the anti-human-PD-1 antibody or antigen binding fragment thereof comprises or consists essentially of

(i) a heavy chain comprising a CDR1 of SEQ ID NO: 1, a CDR2 of SEQ ID NO: 2 and a CDR3 of SEQ ID NO: 4; and (ii) a light chain comprising a CDR1 of SEQ ID NO: 13, a CDR2 of SEQ ID NO: 15 and a CDR3 of SEQ ID NO: 16; or
(i) a heavy chain comprising a CDR1 of SEQ ID NO: 1, a CDR2 of SEQ ID NO: 2 and a CDR3 of SEQ ID NO: 5; and (ii) a light chain comprising a CDR1 of SEQ ID NO: 13, a CDR2 of SEQ ID NO: 15 and a CDR3 of SEQ ID NO: 16; or
(i) a heavy chain comprising a CDR1 of SEQ ID NO: 1, a CDR2 of SEQ ID NO: 2 and a CDR3 of SEQ ID NO: 6; and (ii) a light chain comprising a CDR1 of SEQ ID NO: 13, a CDR2 of SEQ ID NO: 15 and a CDR3 of SEQ ID NO: 16; or
(i) a heavy chain comprising a CDR1 of SEQ ID NO: 1, a CDR2 of SEQ ID NO: 2 and a CDR3 of SEQ ID NO: 7; and (ii) a light chain comprising a CDR1 of SEQ ID NO: 13, a CDR2 of SEQ ID NO: 15 and a CDR3 of SEQ ID NO: 16; or.
(i) a heavy chain comprising a CDR1 of SEQ ID NO: 1, a CDR2 of SEQ ID NO: 2 and a CDR3 of SEQ ID NO: 8; and (ii) a light chain comprising a CDR1 of SEQ ID NO: 13, a CDR2 of SEQ ID NO: 15 and a CDR3 of SEQ ID NO: 16; or
(i) a heavy chain comprising a CDR1 of SEQ ID NO: 1, a CDR2 of SEQ ID NO: 2 and a CDR3 of SEQ ID NO: 9; and (ii) a light chain comprising a CDR1 of SEQ ID NO: 13, a CDR2 of SEQ ID NO: 15 and a CDR3 of SEQ ID NO: 16; or
(i) a heavy chain comprising a CDR1 of SEQ ID NO: 1, a CDR2 of SEQ ID NO: 2 and a CDR3 of SEQ ID NO: 10; and (ii) a light chain comprising a CDR1 of SEQ ID NO: 13, a CDR2 of SEQ ID NO: 15 and a CDR3 of SEQ ID NO: 16; or
(i) a heavy chain comprising a CDR1 of SEQ ID NO: 1, a CDR2 of SEQ ID NO: 2 and a CDR3 of SEQ ID NO: 11; and (ii) a light chain comprising a CDR1 of SEQ ID NO: 13, a CDR2 of SEQ ID NO: 15 and a CDR3 of SEQ ID NO: 16; or
(i) a heavy chain comprising a CDR1 of SEQ ID NO: 1, a CDR2 of SEQ ID NO: 2 and a CDR3 of SEQ ID NO: 4; and (ii) a light chain comprising a CDR1 of SEQ ID NO: 14, a CDR2 of SEQ ID NO: 15 and a CDR3 of SEQ ID NO: 16; or
(i) a heavy chain comprising a CDR1 of SEQ ID NO: 1, a CDR2 of SEQ ID NO: 2 and a CDR3 of SEQ ID NO: 5; and (ii) a light chain comprising a CDR1 of SEQ ID NO: 14, a CDR2 of SEQ ID NO: 15 and a CDR3 of SEQ ID NO: 16; or
(i) a heavy chain comprising a CDR1 of SEQ ID NO: 1, a CDR2 of SEQ ID NO: 2 and a CDR3 of SEQ ID NO: 6; and (ii) a light chain comprising a CDR1 of SEQ ID NO: 14, a CDR2 of SEQ ID NO: 15 and a CDR3 of SEQ ID NO: 16; or
(i) a heavy chain comprising a CDR1 of SEQ ID NO: 1, a CDR2 of SEQ ID NO: 2 and a CDR3 of SEQ ID NO: 7; and (ii) a light chain comprising a CDR1 of SEQ ID NO: 14, a CDR2 of SEQ ID NO: 15 and a CDR3 of SEQ ID NO: 16; or
(i) a heavy chain comprising a CDR1 of SEQ ID NO: 1, a CDR2 of SEQ ID NO: 2 and a CDR3 of SEQ ID NO: 8; and (ii) a light chain comprising a CDR1 of SEQ ID NO: 14, a CDR2 of SEQ ID NO: 15 and a CDR3 of SEQ ID NO: 16; or
(i) a heavy chain comprising a CDR1 of SEQ ID NO: 1, a CDR2 of SEQ ID NO: 2 and a CDR3 of SEQ ID NO: 9; and (ii) a light chain comprising a CDR1 of SEQ ID NO: 14, a CDR2 of SEQ ID NO: 15 and a CDR3 of SEQ ID NO: 16; or
(i) a heavy chain comprising a CDR1 of SEQ ID NO: 1, a CDR2 of SEQ ID NO: 2 and a CDR3 of SEQ ID NO: 10; and (ii) a light chain comprising a CDR1 of SEQ ID NO: 14, a CDR2 of SEQ ID NO: 15 and a CDR3 of SEQ ID NO: 16; or
(i) a heavy chain comprising a CDR1 of SEQ ID NO: 1, a CDR2 of SEQ ID NO: 2 and a CDR3 of SEQ ID NO: 11; and (ii) a light chain comprising a CDR1 of SEQ ID NO: 14, a CDR2 of SEQ ID NO: 15 and a CDR3 of SEQ ID NO: 16.

### Framework

In one embodiment, the anti-PD1 antibody or antigen binding fragment according to the invention comprises framework regions, in particular heavy chain variable region framework regions (HFR) HFR1, HFR2, HFR3 and HFR4 and light chain variable region framework regions (LFR) LFR1, LFR2, LFR3 and LFR4. Preferably, the anti-PD1 antibody or antigen binding fragment according to the invention comprises human or humanized framework regions. A “human acceptor framework” for the purposes herein is a framework comprising the amino acid sequence of a light chain variable domain (VL) framework or a heavy chain variable domain (VH) framework derived from a human immunoglobulin framework or a human consensus framework, as defined below. A human acceptor framework derived from a human immunoglobulin framework or a human consensus framework may comprise the same amino acid sequence thereof, or it may contain amino acid sequence changes. In some embodiments, the number of amino acid changes are 10 or less, 9 or less, 8 or less, 7 or less, 6 or less, 5 or less, 4 or less, 3 or less, or 2 or less. In some embodiments, the VL acceptor human framework is identical in sequence to the VL human immunoglobulin framework sequence or human consensus framework sequence. A “human consensus framework” is a framework which represents the most commonly occurring amino acid residues in a selection of human immunoglobulin VL or VH framework sequences.

Particularly, the anti-PD1 antibody or antigen binding fragment comprises heavy chain variable region framework regions (HFR) HFR1, HFR2, HFR3 and HFR4 comprising an amino acid sequence of SEQ ID NOs: 41, 42, 43 and 44, respectively, optionally with one, two or three modification(s) selected from substitution(s), addition(s), deletion(s) and any combination thereof at any position but positions 27, 29 and 32 of HFR3, i.e., of SEQ ID NO: 43. Preferably, the anti-PD1 antibody or antigen binding fragment comprises HFR1 of SEQ ID NO: 41, HFR2 of SEQ ID NO: 42, HFR3 of SEQ ID NO: 43 and HFR4 of SEQ ID NO: 44.

Alternatively or additionally, the anti-PD1 antibody or antigen binding fragment comprises light chain variable region framework regions (LFR) LFR1, LFR2, LFR3 and LFR4 comprising an amino acid sequence of SEQ ID NOs: 45, 46, 47 and 48, respectively, optionally with one, two or three modification(s) selected from substitution(s), addition(s), deletion(s) and any combination thereof. Preferably, the humanized anti-PD1 antibody or antigen binding fragment comprises LFR1 of SEQ ID NO: 45, LFR2 of SEQ ID NO: 46, LFR3 of SEQ ID NO: 47 and LFR4 of SEQ ID NO: 48.

### VH-VL

The VL and VH domain of the anti hPD1 antibody comprised in the bifunctional molecule according to the invention may comprise four framework regions interrupted by three complementary determining regions preferably operably linked in the following order: FR1-CDR1-FR2-CDR2-FR3-CDR3-FR4 (from amino terminus to carboxy terminus).

In a first embodiment, the anti-human-PD-1 humanized antibody or antigen binding fragment thereof comprised in the bifunctional molecule comprises:

(a) a heavy chain variable region (VH) comprising or consisting of an amino acid sequence of SEQ ID NO: 17, wherein X1 is D or E and X2 is selected from the group consisting of T, H, A, Y, N, E and S preferably in the group consisting of H, A, Y, N, E; optionally with one, two or three modification(s) selected from substitution(s), addition(s), deletion(s) and any combination thereof at any position but positions 7, 16, 17, 20, 33, 38, 43, 46, 62, 63, 65, 69, 73, 76, 78, 80, 84, 85, 88, 93, 95, 96, 97, 98, 100, 101, 105, 106 and 112 of SEQ ID NO: 17;
(b) a light chain variable region (VL) comprising or consisting of an amino acid sequence of SEQ ID NO: 26, wherein X is G or T, optionally with one, two or three modification(s) selected from substitution(s), addition(s), deletion(s) and any combination thereof at any position but positions 3, 4, 7, 14, 17, 18, 28, 29, 33, 34, 39, 42, 44, 50, 81, 88, 94, 97, 99 and 105 of SEQ ID NO: 26.

In a second embodiment, the anti-human-PD-1 humanized antibody or antigen binding fragment thereof comprised in the bifunctional molecule comprises:

(a) a heavy chain variable region (VH) comprising or consisting of an amino acid sequence of SEQ ID NO: 17, wherein either X1 is D and X2 is selected from the group consisting of T, H, A, Y, N, E, preferably in the group consisting of H, A, Y, N, E; or X1 is E and X2 is selected from the group consisting of T, H, A, Y, N, E and S preferably in the group consisting of H, A, Y, N, E and S; optionally with one, two or three modification(s) selected from substitution(s), addition(s), deletion(s) and any combination thereof at any position but positions 7, 16, 17, 20, 33, 38, 43, 46, 62, 63, 65, 69, 73, 76, 78, 80, 84, 85, 88, 93, 95, 96, 97, 98, 100, 101, 105, 106 and 112 of SEQ ID NO: 17;
(b) a light chain variable region (VL) comprising or consisting of an amino acid sequence of SEQ ID NO: 26, wherein X is G or T, optionally with one, two or three modification(s) selected from substitution(s), addition(s), deletion(s) and any combination thereof at any position but positions 3, 4, 7, 14, 17, 18, 28, 29, 33, 34, 39, 42, 44, 50, 81, 88, 94, 97, 99 and 105 of SEQ ID NO: 26.

In a third embodiment, the anti-human-PD-1 humanized antibody or antigen binding fragment thereof comprised in the bifunctional molecule comprises:

(a) a heavy chain variable region (VH) comprising or consisting of an amino acid sequence of SEQ ID NO: 17, wherein X1 is E and X2 is selected from the group consisting of T, H, A, Y, N, E and S preferably in the group consisting of H, A, Y, N, E and 5; optionally with one, two or three modification(s) selected from substitution(s), addition(s), deletion(s) and any combination thereof at any position but positions 7, 16, 17, 20, 33, 38, 43, 46, 62, 63, 65, 69, 73, 76, 78, 80, 84, 85, 88, 93, 95, 96, 97, 98, 100, 101, 105, 106 and 112 of SEQ ID NO: 17;
(b) a light chain variable region (VL) comprising or consisting of an amino acid sequence of SEQ ID NO: 26, wherein X is G or T, optionally with one, two or three modification(s) selected from substitution(s), addition(s), deletion(s) and any combination thereof at any position but positions 3, 4, 7, 14, 17, 18, 28, 29, 33, 34, 39, 42, 44, 50, 81, 88, 94, 97, 99 and 105 of SEQ ID NO: 26.

In another embodiment, the anti-human-PD-1 humanized antibody or antigen binding fragment thereof comprised in the bifunctional molecule comprises:

(a) a heavy chain variable region (VH) comprising or consisting of an amino acid sequence of SEQ ID NO: 18, 19, 20, 21, 22, 23, 24 or 25, optionally with one, two or three modification(s) selected from substitution(s), addition(s), deletion(s) and any combination thereof at any position but positions 7, 16, 17, 20, 33, 38, 43, 46, 62, 63, 65, 69, 73, 76, 78, 80, 84, 85, 88, 93, 95, 96, 97, 98, 100, 101, 105, 106 and 112 of SEQ ID NO: 18, 19, 20, 21, 22, 23, 24 or 25, respectively;
(b) a light chain variable region (VL) comprising or consisting of an amino acid sequence of SEQ ID NO: 27 or SEQ ID NO: 28, optionally with one, two or three modification(s) selected from substitution(s), addition(s), deletion(s) and any combination thereof at any position positions 3, 4, 7, 14, 17, 18, 28, 29, 33, 34, 39, 42, 44, 50, 81, 88, 94, 97, 99 and 105 of SEQ ID NO: 27 or SEQ ID NO: 28.

In another embodiment, the anti-human-PD-1 humanized antibody or antigen binding fragment thereof comprised in the bifunctional molecule comprises:

(a) a heavy chain variable region (VH) comprising or consisting of an amino acid sequence of SEQ ID NO: 18 optionally with one, two or three modification(s) selected from substitution(s), addition(s), deletion(s) and any combination thereof at any position but positions 7, 16, 17, 20, 33, 38, 43, 46, 62, 63, 65, 69, 73, 76, 78, 80, 84, 85, 88, 93, 95, 96, 97, 98, 100, 101, 105, 106 and 112 of SEQ ID NO: 18; and (b) a light chain variable region (VL) comprising or consisting of an amino acid sequence of SEQ ID NO: 27, optionally with one, two or three modification(s) selected from substitution(s), addition(s), deletion(s) and any combination thereof at any position but positions 3, 4, 7, 14, 17, 18, 28, 29, 33, 34, 39, 42, 44, 50, 81, 88, 94, 97, 99 and 105 of SEQ ID NO: 27; or
(a) a heavy chain variable region (VH) comprising or consisting of an amino acid sequence of SEQ ID NO: 19 optionally with one, two or three modification(s) selected from substitution(s), addition(s), deletion(s) and any combination thereof at any position but positions 7, 16, 17, 20, 33, 38, 43, 46, 62, 63, 65, 69, 73, 76, 78, 80, 84, 85, 88, 93, 95, 96, 97, 98, 100, 101, 105, 106 and 112 of SEQ ID NO: 19; and (b) a light chain variable region (VL) comprising or consisting of an amino acid sequence of SEQ ID NO: 27, optionally with one, two or three modification(s) selected from substitution(s), addition(s), deletion(s) and any combination thereof at any position but positions 3, 4, 7, 14, 17, 18, 28, 29, 33, 34, 39, 42, 44, 50, 81, 88, 94, 97, 99 and 105 of SEQ ID NO: 27; or
(a) a heavy chain variable region (VH) comprising or consisting of an amino acid sequence of SEQ ID NO: 20 optionally with one, two or three modification(s) selected from substitution(s), addition(s), deletion(s) and any combination thereof at any position but positions 7, 16, 17, 20, 33, 38, 43, 46, 62, 63, 65, 69, 73, 76, 78, 80, 84, 85, 88, 93, 95, 96, 97, 98, 100, 101, 105, 106 and 112 of SEQ ID NO: 20; and (b) a light chain variable region (VL) comprising or consisting of an amino acid sequence of SEQ ID NO: 27, optionally with one, two or three modification(s) selected from substitution(s), addition(s), deletion(s) and any combination thereof at any position but positions 3, 4, 7, 14, 17, 18, 28, 29, 33, 34, 39, 42, 44, 50, 81, 88, 94, 97, 99 and 105 of SEQ ID NO: 27; or
(a) a heavy chain variable region (VH) comprising or consisting of an amino acid sequence of SEQ ID NO: 21 optionally with one, two or three modification(s) selected from substitution(s), addition(s), deletion(s) and any combination thereof at any position but positions 7, 16, 17, 20, 33, 38, 43, 46, 62, 63, 65, 69, 73, 76, 78, 80, 84, 85, 88, 93, 95, 96, 97, 98, 100, 101, 105, 106 and 112 of SEQ ID NO: 21; and (b) a light chain variable region (VL) comprising or consisting of an amino acid sequence of SEQ ID NO: 27, optionally with one, two or three modification(s) selected from substitution(s), addition(s), deletion(s) and any combination thereof at any position but positions 3, 4, 7, 14, 17, 18, 28, 29, 33, 34, 39, 42, 44, 50, 81, 88, 94, 97, 99 and 105 of SEQ ID NO: 27; or
(a) a heavy chain variable region (VH) comprising or consisting of an amino acid sequence of SEQ ID NO: 22 optionally with one, two or three modification(s) selected from substitution(s), addition(s), deletion(s) and any combination thereof at any position but positions 7, 16, 17, 20, 33, 38, 43, 46, 62, 63, 65, 69, 73, 76, 78, 80, 84, 85, 88, 93, 95, 96, 97, 98, 100, 101, 105, 106 and 112 of SEQ ID NO: 22; and (b) a light chain variable region (VL) comprising or consisting of an amino acid sequence of SEQ ID NO: 27, optionally with one, two or three modification(s) selected from substitution(s), addition(s), deletion(s) and any combination thereof at any position but positions 3, 4, 7, 14, 17, 18, 28, 29, 33, 34, 39, 42, 44, 50, 81, 88, 94, 97, 99 and 105 of SEQ ID NO: 27; or
(a) a heavy chain variable region (VH) comprising or consisting of an amino acid sequence of SEQ ID NO: 23 optionally with one, two or three modification(s) selected from substitution(s), addition(s), deletion(s) and any combination thereof at any position but positions 7, 16, 17, 20, 33, 38, 43, 46, 62, 63, 65, 69, 73, 76, 78, 80, 84, 85, 88, 93, 95, 96, 97, 98, 100, 101, 105, 106 and 112 of SEQ ID NO: 23; and (b) a light chain variable region (VL) comprising or consisting of an amino acid sequence of SEQ ID NO: 27, optionally with one, two or three modification(s) selected from substitution(s), addition(s), deletion(s) and any combination thereof at any position but positions 3, 4, 7, 14, 17, 18, 28, 29, 33, 34, 39, 42, 44, 50, 81, 88, 94, 97, 99 and 105 of SEQ ID NO: 27; or
(a) a heavy chain variable region (VH) comprising or consisting of an amino acid sequence of SEQ ID NO: 24 optionally with one, two or three modification(s) selected from substitution(s), addition(s), deletion(s) and any combination thereof at any position but positions 7, 16, 17, 20, 33, 38, 43, 46, 62, 63, 65, 69, 73, 76, 78, 80, 84, 85, 88, 93, 95, 96, 97, 98, 100, 101, 105, 106 and 112 of SEQ ID NO: 24; and (b) a light chain variable region (VL) comprising or consisting of an amino acid sequence of SEQ ID NO: 27, optionally with one, two or three modification(s) selected from substitution(s), addition(s), deletion(s) and any combination thereof at any position but positions 3, 4, 7, 14, 17, 18, 28, 29, 33, 34, 39, 42, 44, 50, 81, 88, 94, 97, 99 and 105 of SEQ ID NO: 27; or
(a) a heavy chain variable region (VH) comprising or consisting of an amino acid sequence of SEQ ID NO: 25 optionally with one, two or three modification(s) selected from substitution(s), addition(s), deletion(s) and any combination thereof at any position but positions 7, 16, 17, 20, 33, 38, 43, 46, 62, 63, 65, 69, 73, 76, 78, 80, 84, 85, 88, 93, 95, 96, 97, 98, 100, 101, 105, 106 and 112 of SEQ ID NO: 25; and (b) a light chain variable region (VL) comprising or consisting of an amino acid sequence of SEQ ID NO: 27, optionally with one, two or three modification(s) selected from substitution(s), addition(s), deletion(s) and any combination thereof at any position but positions 3, 4, 7, 14, 17, 18, 28, 29, 33, 34, 39, 42, 44, 50, 81, 88, 94, 97, 99 and 105 of SEQ ID NO: 27; or
(a) a heavy chain variable region (VH) comprising or consisting of an amino acid sequence of SEQ ID NO: 18 optionally with one, two or three modification(s) selected from substitution(s), addition(s), deletion(s) and any combination thereof at any position but positions 7, 16, 17, 20, 33, 38, 43, 46, 62, 63, 65, 69, 73, 76, 78, 80, 84, 85, 88, 93, 95, 96, 97, 98, 100, 101, 105, 106 and 112 of SEQ ID NO: 18; and (b) a light chain variable region (VL) comprising or consisting of an amino acid sequence of SEQ ID NO: 28, optionally with one, two or three modification(s) selected from substitution(s), addition(s), deletion(s) and any combination thereof at any position but positions 3, 4, 7, 14, 17, 18, 28, 29, 33, 34, 39, 42, 44, 50, 81, 88, 94, 97, 99 and 105 of SEQ ID NO: 28; or
(a) a heavy chain variable region (VH) comprising or consisting of an amino acid sequence of SEQ ID NO: 19 optionally with one, two or three modification(s) selected from substitution(s), addition(s), deletion(s) and any combination thereof at any position but positions 7, 16, 17, 20, 33, 38, 43, 46, 62, 63, 65, 69, 73, 76, 78, 80, 84, 85, 88, 93, 95, 96, 97, 98, 100, 101, 105, 106 and 112 of SEQ ID NO: 19; and (b) a light chain variable region (VL) comprising or consisting of an amino acid sequence of SEQ ID NO: 28, optionally with one, two or three modification(s) selected from substitution(s), addition(s), deletion(s) and any combination thereof at any position but positions 3, 4, 7, 14, 17, 18, 28, 29, 33, 34, 39, 42, 44, 50, 81, 88, 94, 97, 99 and 105 of SEQ ID NO: 28; or
(a) a heavy chain variable region (VH) comprising or consisting of an amino acid sequence of SEQ ID NO: 20 optionally with one, two or three modification(s) selected from substitution(s), addition(s), deletion(s) and any combination thereof at any position but positions 7, 16, 17, 20, 33, 38, 43, 46, 62, 63, 65, 69, 73, 76, 78, 80, 84, 85, 88, 93, 95, 96, 97, 98, 100, 101, 105, 106 and 112 of SEQ ID NO: 20; and (b) a light chain variable region (VL) comprising or consisting of an amino acid sequence of SEQ ID NO: 28, optionally with one, two or three modification(s) selected from substitution(s), addition(s), deletion(s) and any combination thereof at any position but positions 3, 4, 7, 14, 17, 18, 28, 29, 33, 34, 39, 42, 44, 50, 81, 88, 94, 97, 99 and 105 of SEQ ID NO: 28; or
(a) a heavy chain variable region (VH) comprising or consisting of an amino acid sequence of SEQ ID NO: 21 optionally with one, two or three modification(s) selected from substitution(s), addition(s), deletion(s) and any combination thereof at any position but positions 7, 16, 17, 20, 33, 38, 43, 46, 62, 63, 65, 69, 73, 76, 78, 80, 84, 85, 88, 93, 95, 96, 97, 98, 100, 101, 105, 106 and 112 of SEQ ID NO: 21; and (b) a light chain variable region (VL) comprising or consisting of an amino acid sequence of SEQ ID NO: 28, optionally with one, two or three modification(s) selected from substitution(s), addition(s), deletion(s) and any combination thereof at any position but positions 3, 4, 7, 14, 17, 18, 28, 29, 33, 34, 39, 42, 44, 50, 81, 88, 94, 97, 99 and 105 of SEQ ID NO: 28; or
(a) a heavy chain variable region (VH) comprising or consisting of an amino acid sequence of SEQ ID NO: 22 optionally with one, two or three modification(s) selected from substitution(s), addition(s), deletion(s) and any combination thereof at any position but positions 7, 16, 17, 20, 33, 38, 43, 46, 62, 63, 65, 69, 73, 76, 78, 80, 84, 85, 88, 93, 95, 96, 97, 98, 100, 101, 105, 106 and 112 of SEQ ID NO: 22; and (b) a light chain variable region (VL) comprising or consisting of an amino acid sequence of SEQ ID NO: 28, optionally with one, two or three modification(s) selected from substitution(s), addition(s), deletion(s) and any combination thereof at any position but positions 3, 4, 7, 14, 17, 18, 28, 29, 33, 34, 39, 42, 44, 50, 81, 88, 94, 97, 99 and 105 of SEQ ID NO: 28; or
(a) a heavy chain variable region (VH) comprising or consisting of an amino acid sequence of SEQ ID NO: 23 optionally with one, two or three modification(s) selected from substitution(s), addition(s), deletion(s) and any combination thereof at any position but positions 7, 16, 17, 20, 33, 38, 43, 46, 62, 63, 65, 69, 73, 76, 78, 80, 84, 85, 88, 93, 95, 96, 97, 98, 100, 101, 105, 106 and 112 of SEQ ID NO: 23; and (b) a light chain variable region (VL) comprising or consisting of an amino acid sequence of SEQ ID NO: 28, optionally with one, two or three modification(s) selected from substitution(s), addition(s), deletion(s) and any combination thereof at any position but positions 3, 4, 7, 14, 17, 18, 28, 29, 33, 34, 39, 42, 44, 50, 81, 88, 94, 97, 99 and 105 of SEQ ID NO: 28; or
(a) a heavy chain variable region (VH) comprising or consisting of an amino acid sequence of SEQ ID NO: 24 optionally with one, two or three modification(s) selected from substitution(s), addition(s), deletion(s) and any combination thereof at any position but positions 7, 16, 17, 20, 33, 38, 43, 46, 62, 63, 65, 69, 73, 76, 78, 80, 84, 85, 88, 93, 95, 96, 97, 98, 100, 101, 105, 106 and 112 of SEQ ID NO: 24; and (b) a light chain variable region (VL) comprising or consisting of an amino acid sequence of SEQ ID NO: 28, optionally with one, two or three modification(s) selected from substitution(s), addition(s), deletion(s) and any combination thereof at any position but positions 3, 4, 7, 14, 17, 18, 28, 29, 33, 34, 39, 42, 44, 50, 81, 88, 94, 97, 99 and 105 of SEQ ID NO: 28; or
(a) a heavy chain variable region (VH) comprising or consisting of an amino acid sequence of SEQ ID NO: 25 optionally with one, two or three modification(s) selected from substitution(s), addition(s), deletion(s) and any combination thereof at any position but positions 7, 16, 17, 20, 33, 38, 43, 46, 62, 63, 65, 69, 73, 76, 78, 80, 84, 85, 88, 93, 95, 96, 97, 98, 100, 101, 105, 106 and 112 of SEQ ID NO: 25; and (b) a light chain variable region (VL) comprising or consisting of an amino acid sequence of SEQ ID NO: 28, optionally with one, two or three modification(s) selected from substitution(s), addition(s), deletion(s) and any combination thereof at any position but positions 3, 4, 7, 14, 17, 18, 28, 29, 33, 34, 39, 42, 44, 50, 81, 88, 94, 97, 99 and 105 of SEQ ID NO: 28.

In a particular aspect, the modifications are substitutions, in particular conservative substitutions.

### CH-CL

In one embodiment, the heavy chain (CH) and the light chain (CL) comprises the VL and VH sequences as described hereabove.

In a particular embodiment, the anti-human-PD-1 antibody or antigen binding fragment thereof comprised in the bifunctional molecule comprises:

(a) a heavy chain comprising or consisting of an amino acid sequence selected from the group consisting of SEQ ID NO: 29, 30, 31, 32, 33, 34, 35 or 36, optionally with one, two or three modification(s) selected from substitution(s), addition(s), deletion(s) and any combination thereof at any position but positions 7, 16, 17, 20, 33, 38, 43, 46, 62, 63, 65, 69, 73, 76, 78, 80, 84, 85, 88, 93, 95, 96, 97, 98, 100, 101, 105, 106 and 112 of SEQ ID NO: 29, 30, 31, 32, 33, 34, 35 or 36, respectively, and
(b) a light chain comprising or consisting of an amino acid sequence of SEQ ID NO: 37 or SEQ ID NO: 38, optionally with one, two or three modification(s) selected from substitution(s), addition(s), deletion(s) and any combination thereof at any position but positions 3, 4, 7, 14, 17, 18, 28, 29, 33, 34, 39, 42, 44, 50, 81, 88, 94, 97, 99 and 105 of SEQ ID NO: 37 or SEQ ID NO: 38.

In another embodiment, the anti-human-PD-1 humanized antibody or antigen binding fragment thereof comprised in the bifunctional molecule comprises:

(a) a heavy chain comprising or consisting of an amino acid sequence selected from the group consisting of SEQ ID NO: 29, optionally with one, two or three modification(s) selected from substitution(s), addition(s), deletion(s) and any combination thereof at any position but positions 7, 16, 17, 20, 33, 38, 43, 46, 62, 63, 65, 69, 73, 76, 78, 80, 84, 85, 88, 93, 95, 96, 97, 98, 100, 101, 105, 106 and 112 of SEQ ID NO: 29, and (b) a light chain comprising or consisting of an amino acid sequence of SEQ ID NO: 37, optionally with one, two or three modification(s) selected from substitution(s), addition(s), deletion(s) and any combination thereof at any position but positions 3, 4, 7, 14, 17, 18, 28, 29, 33, 34, 39, 42, 44, 50, 81, 88, 94, 97, 99 and 105 of SEQ ID NO: 37; or
(a) a heavy chain comprising or consisting of an amino acid sequence selected from the group consisting of SEQ ID NO: 30, optionally with one, two or three modification(s) selected from substitution(s), addition(s), deletion(s) and any combination thereof at any position but positions 7, 16, 17, 20, 33, 38, 43, 46, 62, 63, 65, 69, 73, 76, 78, 80, 84, 85, 88, 93, 95, 96, 97, 98, 100, 101, 105, 106 and 112 of SEQ ID NO: 30, and (b) a light chain comprising or consisting of an amino acid sequence of SEQ ID NO: 37, optionally with one, two or three modification(s) selected from substitution(s), addition(s), deletion(s) and any combination thereof at any position but positions 3, 4, 7, 14, 17, 18, 28, 29, 33, 34, 39, 42, 44, 50, 81, 88, 94, 97, 99 and 105 of SEQ ID NO: 37; or
(a) a heavy chain comprising or consisting of an amino acid sequence selected from the group consisting of SEQ ID NO: 31, optionally with one, two or three modification(s) selected from substitution(s), addition(s), deletion(s) and any combination thereof at any position but positions 7, 16, 17, 20, 33, 38, 43, 46, 62, 63, 65, 69, 73, 76, 78, 80, 84, 85, 88, 93, 95, 96, 97, 98, 100, 101, 105, 106 and 112 of SEQ ID NO: 31, and (b) a light chain comprising or consisting of an amino acid sequence of SEQ ID NO: 37, optionally with one, two or three modification(s) selected from substitution(s), addition(s), deletion(s) and any combination thereof at any position but positions 3, 4, 7, 14, 17, 18, 28, 29, 33, 34, 39, 42, 44, 50, 81, 88, 94, 97, 99 and 105 of SEQ ID NO: 37; or
(a) a heavy chain comprising or consisting of an amino acid sequence selected from the group consisting of SEQ ID NO: 32, optionally with one, two or three modification(s) selected from substitution(s), addition(s), deletion(s) and any combination thereof at any position but positions 7, 16, 17, 20, 33, 38, 43, 46, 62, 63, 65, 69, 73, 76, 78, 80, 84, 85, 88, 93, 95, 96, 97, 98, 100, 101, 105, 106 and 112 of SEQ ID NO: 32, and (b) a light chain comprising or consisting of an amino acid sequence of SEQ ID NO: 37, optionally with one, two or three modification(s) selected from substitution(s), addition(s), deletion(s) and any combination thereof at any position but positions 3, 4, 7, 14, 17, 18, 28, 29, 33, 34, 39, 42, 44, 50, 81, 88, 94, 97, 99 and 105 of SEQ ID NO: 37; or
(a) a heavy chain comprising or consisting of an amino acid sequence selected from the group consisting of SEQ ID NO: 33, optionally with one, two or three modification(s) selected from substitution(s), addition(s), deletion(s) and any combination thereof at any position but positions 7, 16, 17, 20, 33, 38, 43, 46, 62, 63, 65, 69, 73, 76, 78, 80, 84, 85, 88, 93, 95, 96, 97, 98, 100, 101, 105, 106 and 112 of SEQ ID NO: 33, and (b) a light chain comprising or consisting of an amino acid sequence of SEQ ID NO: 37, optionally with one, two or three modification(s) selected from substitution(s), addition(s), deletion(s) and any combination thereof at any position but positions 3, 4, 7, 14, 17, 18, 28, 29, 33, 34, 39, 42, 44, 50, 81, 88, 94, 97, 99 and 105 of SEQ ID NO: 37; or
(a) a heavy chain comprising or consisting of an amino acid sequence selected from the group consisting of SEQ ID NO: 34, optionally with one, two or three modification(s) selected from substitution(s), addition(s), deletion(s) and any combination thereof at any position but positions 7, 16, 17, 20, 33, 38, 43, 46, 62, 63, 65, 69, 73, 76, 78, 80, 84, 85, 88, 93, 95, 96, 97, 98, 100, 101, 105, 106 and 112 of SEQ ID NO: 34, and (b) a light chain comprising or consisting of an amino acid sequence of SEQ ID NO: 37, optionally with one, two or three modification(s) selected from substitution(s), addition(s), deletion(s) and any combination thereof at any position but positions 3, 4, 7, 14, 17, 18, 28, 29, 33, 34, 39, 42, 44, 50, 81, 88, 94, 97, 99 and 105 of SEQ ID NO: 37; or
(a) a heavy chain comprising or consisting of an amino acid sequence selected from the group consisting of SEQ ID NO: 35, optionally with one, two or three modification(s) selected from substitution(s), addition(s), deletion(s) and any combination thereof at any position but positions 7, 16, 17, 20, 33, 38, 43, 46, 62, 63, 65, 69, 73, 76, 78, 80, 84, 85, 88, 93, 95, 96, 97, 98, 100, 101, 105, 106 and 112 of SEQ ID NO: 35, and (b) a light chain comprising or consisting of an amino acid sequence of SEQ ID NO: 37, optionally with one, two or three modification(s) selected from substitution(s), addition(s), deletion(s) and any combination thereof at any position but positions 3, 4, 7, 14, 17, 18, 28, 29, 33, 34, 39, 42, 44, 50, 81, 88, 94, 97, 99 and 105 of SEQ ID NO: 37; or
(a) a heavy chain comprising or consisting of an amino acid sequence selected from the group consisting of SEQ ID NO: 36, optionally with one, two or three modification(s) selected from substitution(s), addition(s), deletion(s) and any combination thereof at any position but positions 7, 16, 17, 20, 33, 38, 43, 46, 62, 63, 65, 69, 73, 76, 78, 80, 84, 85, 88, 93, 95, 96, 97, 98, 100, 101, 105, 106 and 112 of SEQ ID NO: 36, and (b) a light chain comprising or consisting of an amino acid sequence of SEQ ID NO: 37, optionally with one, two or three modification(s) selected from substitution(s), addition(s), deletion(s) and any combination thereof at any position but positions 3, 4, 7, 14, 17, 18, 28, 29, 33, 34, 39, 42, 44, 50, 81, 88, 94, 97, 99 and 105 of SEQ ID NO: 37; or
(a) a heavy chain comprising or consisting of an amino acid sequence selected from the group consisting of SEQ ID NO: 29, optionally with one, two or three modification(s) selected from substitution(s), addition(s), deletion(s) and any combination thereof at any position but positions 7, 16, 17, 20, 33, 38, 43, 46, 62, 63, 65, 69, 73, 76, 78, 80, 84, 85, 88, 93, 95, 96, 97, 98, 100, 101, 105, 106 and 112 of SEQ ID NO: 29, and (b) a light chain comprising or consisting of an amino acid sequence of SEQ ID NO: 38, optionally with one, two or three modification(s) selected from substitution(s), addition(s), deletion(s) and any combination thereof at any position but positions 3, 4, 7, 14, 17, 18, 28, 29, 33, 34, 39, 42, 44, 50, 81, 88, 94, 97, 99 and 105 of SEQ ID NO: 38; or
(a) a heavy chain comprising or consisting of an amino acid sequence selected from the group consisting of SEQ ID NO: 30, optionally with one, two or three modification(s) selected from substitution(s), addition(s), deletion(s) and any combination thereof at any position but positions 7, 16, 17, 20, 33, 38, 43, 46, 62, 63, 65, 69, 73, 76, 78, 80, 84, 85, 88, 93, 95, 96, 97, 98, 100, 101, 105, 106 and 112 of SEQ ID NO: 30, and (b) a light chain comprising or consisting of an amino acid sequence of SEQ ID NO: 38, optionally with one, two or three modification(s) selected from substitution(s), addition(s), deletion(s) and any combination thereof at any position but positions 3, 4, 7, 14, 17, 18, 28, 29, 33, 34, 39, 42, 44, 50, 81, 88, 94, 97, 99 and 105 of SEQ ID NO: 38; or
(a) a heavy chain comprising or consisting of an amino acid sequence selected from the group consisting of SEQ ID NO: 31, optionally with one, two or three modification(s) selected from substitution(s), addition(s), deletion(s) and any combination thereof at any position but positions 7, 16, 17, 20, 33, 38, 43, 46, 62, 63, 65, 69, 73, 76, 78, 80, 84, 85, 88, 93, 95, 96, 97, 98, 100, 101, 105, 106 and 112 of SEQ ID NO: 31, and (b) a light chain comprising or consisting of an amino acid sequence of SEQ ID NO: 38, optionally with one, two or three modification(s) selected from substitution(s), addition(s), deletion(s) and any combination thereof at any position but positions 3, 4, 7, 14, 17, 18, 28, 29, 33, 34, 39, 42, 44, 50, 81, 88, 94, 97, 99 and 105 of SEQ ID NO: 38; or
(a) a heavy chain comprising or consisting of an amino acid sequence selected from the group consisting of SEQ ID NO: 32, optionally with one, two or three modification(s) selected from substitution(s), addition(s), deletion(s) and any combination thereof at any position but positions 7, 16, 17, 20, 33, 38, 43, 46, 62, 63, 65, 69, 73, 76, 78, 80, 84, 85, 88, 93, 95, 96, 97, 98, 100, 101, 105, 106 and 112 of SEQ ID NO: 32, and (b) a light chain comprising or consisting of an amino acid sequence of SEQ ID NO: 38, optionally with one, two or three modification(s) selected from substitution(s), addition(s), deletion(s) and any combination thereof at any position but positions 3, 4, 7, 14, 17, 18, 28, 29, 33, 34, 39, 42, 44, 50, 81, 88, 94, 97, 99 and 105 of SEQ ID NO: 38; or
(a) a heavy chain comprising or consisting of an amino acid sequence selected from the group consisting of SEQ ID NO: 33, optionally with one, two or three modification(s) selected from substitution(s), addition(s), deletion(s) and any combination thereof at any position but positions 7, 16, 17, 20, 33, 38, 43, 46, 62, 63, 65, 69, 73, 76, 78, 80, 84, 85, 88, 93, 95, 96, 97, 98, 100, 101, 105, 106 and 112 of SEQ ID NO: 33, and (b) a light chain comprising or consisting of an amino acid sequence of SEQ ID NO: 38, optionally with one, two or three modification(s) selected from substitution(s), addition(s), deletion(s) and any combination thereof at any position but positions 3, 4, 7, 14, 17, 18, 28, 29, 33, 34, 39, 42, 44, 50, 81, 88, 94, 97, 99 and 105 of SEQ ID NO: 38; or
(a) a heavy chain comprising or consisting of an amino acid sequence selected from the group consisting of SEQ ID NO: 34, optionally with one, two or three modification(s) selected from substitution(s), addition(s), deletion(s) and any combination thereof at any position but positions 7, 16, 17, 20, 33, 38, 43, 46, 62, 63, 65, 69, 73, 76, 78, 80, 84, 85, 88, 93, 95, 96, 97, 98, 100, 101, 105, 106 and 112 of SEQ ID NO: 34, and (b) a light chain comprising or consisting of an amino acid sequence of SEQ ID NO: 38, optionally with one, two or three modification(s) selected from substitution(s), addition(s), deletion(s) and any combination thereof at any position but positions 3, 4, 7, 14, 17, 18, 28, 29, 33, 34, 39, 42, 44, 50, 81, 88, 94, 97, 99 and 105 of SEQ ID NO: 38; or
(a) a heavy chain comprising or consisting of an amino acid sequence selected from the group consisting of SEQ ID NO: 35, optionally with one, two or three modification(s) selected from substitution(s), addition(s), deletion(s) and any combination thereof at any position but positions 7, 16, 17, 20, 33, 38, 43, 46, 62, 63, 65, 69, 73, 76, 78, 80, 84, 85, 88, 93, 95, 96, 97, 98, 100, 101, 105, 106 and 112 of SEQ ID NO: 35, and (b) a light chain comprising or consisting of an amino acid sequence of SEQ ID NO: 38, optionally with one, two or three modification(s) selected from substitution(s), addition(s), deletion(s) and any combination thereof at any position but positions 3, 4, 7, 14, 17, 18, 28, 29, 33, 34, 39, 42, 44, 50, 81, 88, 94, 97, 99 and 105 of SEQ ID NO: 38; or
(a) a heavy chain comprising or consisting of an amino acid sequence selected from the group consisting of SEQ ID NO: 36, optionally with one, two or three modification(s) selected from substitution(s), addition(s), deletion(s) and any combination thereof at any position but positions 7, 16, 17, 20, 33, 38, 43, 46, 62, 63, 65, 69, 73, 76, 78, 80, 84, 85, 88, 93, 95, 96, 97, 98, 100, 101, 105, 106 and 112 of SEQ ID NO: 36, and (b) a light chain comprising or consisting of an amino acid sequence of SEQ ID NO: 38, optionally with one, two or three modification(s) selected from substitution(s), addition(s), deletion(s) and any combination thereof at any position but positions 3, 4, 7, 14, 17, 18, 28, 29, 33, 34, 39, 42, 44, 50, 81, 88, 94, 97, 99 and 105 of SEQ ID NO: 38.

Preferably, the modifications are substitutions, in particular conservative substitutions.

### Fc and Hinge Region

Several researches to develop therapeutic antibodies had led to engineer the Fc regions to optimize antibody properties allowing the generation of molecules that are better suited to the pharmacology activity required of them. The Fc region of an antibody mediates its serum half-life and effector functions, such as complement-dependent cytotoxicity (CDC), antibody-dependent cellular cytotoxicity (ADCC) and antibody-dependent cell phagocytosis (ADCP). Several mutations located at the interface between the CH2 and CH3 domains, such as T250Q/M428L and M252Y/S254T/T256E+H433K/N434F, have been shown to increase the binding affinity to FcRn and the half-life of IgG1 in vivo. However, there is not always a direct relationship between increased FcRn binding and improved half-life. One approach to improve the efficacy of a therapeutic antibody is to increase its serum persistence, thereby allowing higher circulating levels, less frequent administration and reduced doses. Engineering Fc regions may be desired to either reduce or increase the effector function of the antibody. For antibodies that target cell-surface molecules, especially those on immune cells, abrogating effector functions is required. Conversely, for antibodies intended for oncology use, increasing effector functions may improve the therapeutic activity. The four human IgG isotypes bind the activating Fcγ receptors (FcγRI, FcγRIIa, FcγRIIIa), the inhibitory FcγRIIb receptor, and the first component of complement (C1q) with different affinities, yielding very different effector functions. Binding of IgG to the FcγRs or C1q depends on residues located in the hinge region and the CH2 domain. Two regions of the CH2 domain are critical for FcγRs and C1q binding, and have unique sequences in IgG2 and IgG4.

The antibody according to the invention optionally comprises at least a portion of an immunoglobulin constant region (Fc), typically that of mammalian immunoglobulin, even more preferably a human or humanized immunoglobulin. Preferably, the Fc region is a part of the anti-hPD-1 antibody described herein. The anti-hPD1 antibody or antigen binding fragment thereof comprised in the bifunctional molecule of the invention can include a constant region of an immunoglobulin or a fragment, analog, variant, mutant, or derivative of the constant region. As well known by one skilled in the art, the choice of IgG isotypes of the heavy chain constant domain centers on whether specific functions are required and the need for a suitable in vivo half-life. For example, antibodies designed for selective eradication of cancer cells typically require an active isotype that permits complement activation and effector-mediated cell killing by antibody-dependent cell-mediated cytotoxicity. Both human IgG1 and IgG3 (shorter half-life) isotypes meet these criteria, particularly human IgG1 isotype (wild type and variants). In particular, depending of the IgG isotype of the heavy chain constant domain (particularly human wild type and variants IgG1 isotype), the anti-hPD1 antibody of the invention can be cytotoxic towards cells expressing PD-1 via a CDC, ADCC and/or ADCP mechanism. In fact, the fragment crystallisable (Fc) region interacts with a variety of accessory molecules to mediate indirect effector functions such as antibody-dependent cellular cytotoxicity (ADCC), antibody-dependent cellular phagocytosis (ADCP) and complement-dependent cytotoxicity (CDC).

In preferred embodiments, the constant region is derived from a human immunoglobulin heavy chain, for example, IgG1, IgG2, IgG3, IgG4, or other classes. In a further aspect, the human constant region is selected from the group consisting of IgG1, IgG2, IgG2, IgG3 and IgG4. Preferably, the anti-hPD1 antibody comprised in the bifunctional molecule according to the invention comprises an IgG1 or an IgG4 Fc-region. In a particular aspect, the humanized anti-PD1 antibody comprises a human IgG1 Fc region, optionally with a substitution or a combination of substitutions selected from the group consisting of T250Q/M428L; M252Y/S254T/T256E+H433K/N434F; E233P/L234V/L235A/G236A+A327G/A330S/P331S; E333A; S239D/A330L/1332E; P257I/Q311; K326W/E333S; S239D/1332E/G236A; N297A; L234A/L235A; N297A+M252Y/S254T/T256E; K322A and K444A, preferably selected from the group consisting of N297A optionally in combination with M252Y/S254T/T256E, and L234A/L235A.

More preferably, the humanized anti-hPD1 antibody comprises an IgG4 Fc-region, optionally with a substitution or a combination of substitutions selected from the group consisting of S228P; L234A/L235A, S228P+M252Y/S254T/T256E and K444A. Even more preferably, the anti-hPD1 antibody comprises an IgG4 Fc-region with a S228P that stabilizes the IgG4.

In one embodiment, the anti-PD1 antibody comprises a truncated Fc region or a fragment of the Fc region. In one embodiment, the constant region includes a CH2 domain. In another embodiment, the constant region includes CH2 and CH3 domains or includes hinge-CH2-CH3. Alternatively, the constant region can include all or a portion of the hinge region, the CH2 domain and/or the CH3 domain. Preferably, the constant region contains a CH2 and/or a CH3 domain derived from a human IgG4 heavy chain.

In some embodiments, the constant region contains a CH2 and/or a CH3 domain derived from a human IgG4 heavy chain.

In another embodiment, the constant region includes a CH2 domain and at least a portion of a hinge region. The hinge region can be derived from an immunoglobulin heavy chain, e.g., IgG1, IgG2, IgG3, IgG4, or other classes. Preferably, the hinge region is derived from human IgG1, IgG2, IgG3, IgG4, or other suitable classes, mutated or not. More preferably the hinge region is derived from a human IgG1 heavy chain. In one embodiment, the constant region includes a CH2 domain derived from a first antibody isotype and a hinge region derived from a second antibody isotype. In a specific embodiment, the CH2 domain is derived from a human IgG2 or IgG4 heavy chain, while the hinge region is derived from an altered human IgG1 heavy chain.

In one embodiment, the constant region contains a mutation that reduces affinity for an Fc receptor or reduces Fc effector function. For example, the constant region can contain a mutation that eliminates the glycosylation site within the constant region of an IgG heavy chain.

In another embodiment, the constant region includes a CH2 domain and at least a portion of a hinge region. The hinge region can be derived from an immunoglobulin heavy chain, e.g., IgG1, IgG2, IgG3, IgG4, or other classes. Preferably, the hinge region is derived from human IgG1, IgG2, IgG3, IgG4, or other suitable classes. The IgG1 hinge region has three cysteines, two of which are involved in disulfide bonds between the two heavy chains of the immunoglobulin. These same cysteines permit efficient and consistent disulfide bonding formation between Fc portions. Therefore, a preferred hinge region of the present invention is derived from IgG1, more preferably from human IgG1. In some embodiments, the first cysteine within the human IgG1 hinge region is mutated to another amino acid, preferably serine. The IgG2 isotype hinge region has four disulfide bonds that tend to promote oligomerization and possibly incorrect disulfide bonding during secretion in recombinant systems. A suitable hinge region can be derived from an IgG2 hinge; the first two cysteines are each preferably mutated to another amino acid. The hinge region of IgG4 is known to form interchain disulfide bonds inefficiently. However, a suitable hinge region for the present invention can be derived from the IgG4 hinge region, preferably containing a mutation that enhances correct formation of disulfide bonds between heavy chain-derived moieties (Angal S, et al. (1993) Mol. Immunol., 30:105-8). More preferably the hinge region is derived from a human IgG4 heavy chain.

In one embodiment, the constant region includes a CH2 domain derived from a first antibody isotype and a hinge region derived from a second antibody isotype. In a specific embodiment, the CH2 domain is derived from a human IgG4 heavy chain, while the hinge region is derived from an altered human IgG1 heavy chain.

In accordance with the present invention, the constant region can contain CH2 and/or CH3 domains and a hinge region that are derived from different antibody isotypes, i.e., a hybrid constant region. For example, in one embodiment, the constant region contains CH2 and/or CH3 domains derived from IgG2 or IgG4 and a mutant hinge region derived from IgG1. Alternatively, a mutant hinge region from another IgG subclass is used in a hybrid constant region. For example, a mutant form of the IgG4 hinge that allows efficient disulfide bonding between the two heavy chains can be used. A mutant hinge can also be derived from an IgG2 hinge in which the first two cysteines are each mutated to another amino acid. Assembly of such hybrid constant regions has been described in U.S. Patent Publication No. 20030044423, the disclosure of which is hereby incorporated by reference.

In one embodiment, the constant region can contain CH2 and/or CH3 has one of the mutations described in the Table D below, or any combination thereof.

TABLE D
Suitable human engineered Fc domain of an antibody. Numbering of residues in the heavy chain
constant region is according to EU numbering (Edelman, G.M. et al., Proc. Natl. Acad. USA, 63, 78-85
(1969); www.imgt.org/IMGTScientificChart/Numbering/Hu_IGHGnber.html#refs).

| Engineered Fc | Isotype | Mutations | FcR/C1q Binding | Effector Function |
|---|---|---|---|---|
| hlgG1e1-Fc | IgG1 | T250Q/M428L | Increased binding | Increased half-life |
| | | | to FcRn | |
| hlgG1e2-Fc | IgG1 | M252Y/S254T/T256E + | Increased binding | Increased half-life |
| | | H433K/N434F | to FcRn | |
| hlgG1e3-Fc | IgG1 | E233P/L234V/L235A/ | Reduced binding to | Reduced ADCC |
| | | G236A + | FcγRI | and CDC |
| | | A327G/A330S/P331S | | |
| hlgG1e4-Fc | IgG1 | E333A | Increased binding | Increased ADCC |
| | | | to FcγRIIIa | and CDC |
| hlgG1e5-Fc | IgG1 | S239D/A330L/I332E | Increased binding | Increased ADCC |
| | | | to FcyRIIIa | |
| hlgG1e6-Fc | IgG1 | P257I/Q311 | Increased binding | Unchanged half-life |
| | | | to FcRn | |
| hlgG1e7-Fc | IgG1 | K326W/E333S | Increased binding | Increased CDC |
| | | | to C1q | |
| hlgG1e9-Fc | IgG1 | S239D/I332E/G236A | Increased | Increased macrophage |
| | | | FcγRIIa/FcγRIIb | phagocytosis |
| | | | ratio | |
| hlgG1e9-Fc | IgG1 | N297A | Reduced binding to | Reduced ADCC |
| | | | FcγRI | and CDC |
| hlgG1e9-Fc | IgG1 | LALA (L234A/L235A) | Reduced binding to | Reduced ADCC |
| | | | FcγRI | and CDC |
| hlgG1e10-Fc | IgG1 | N297A + YTE | Reduced binding to | Reduced ADCC |
| | | (N298A + | FcγRI | and CDC |
| | | M252Y/S254T/T256E) | Increased binding | Increased half-life |
| | | | to FcRn | |
| hlgG1e11-Fc | IgG1 | K322A | Reduced binding | Reduced CDC |
| | | | to C1q | |
| hlgG1e112-Fc | IgG4 | K444A | | Abolish cleavage of |
| | | | | the C-terminal lysine |
| | | | | of the antibody |
| hIgG4e1-Fc | IgG4 | S228P | — | Reduced Fab-arm |
| | | | | exchange |
| hIgG4e1-Fc | IgG4 | LALA (L234A/L235A) | Increased binding | Increased half-life |
| | | | to FcRn | |
| hIgG4e2-Fc | IgG4 | S228P + YTE (S228P + | Increased binding | Reduced Fab-arm |
| | | M252Y/S254T/T256E) | to FcRn | exchange |
| | | | | Increased half-life |
| hIgG4e3-Fc | IgG4 | K444A | | Abolish cleavage of |
| | | | | the C-terminal lysine |
| | | | | of the antibody |

In certain embodiments, amino acid modifications may be introduced into the Fc region of an antibody provided herein to generate an Fc region variant. In certain embodiments, the Fc region variant possesses some, but not all, effector functions. Such antibodies may be useful, for example, in applications in which the half-life of the antibody in vivo is important, yet certain effector functions are unnecessary or deleterious. Examples of effector functions include complement-dependent cytotoxicity (CDC) and antibody-directed complement-mediated cytotoxicity (ADCC). Numerous substitutions or substitutions or deletions with altered effector function are known in the art.

In one embodiment, the constant region contains a mutation that reduces affinity for an Fc receptor or reduces Fc effector function. For example, the constant region can contain a mutation that eliminates the glycosylation site within the constant region of an IgG heavy chain. Preferably, the CH2 domain contains a mutation that eliminates the glycosylation site within the CH2 domain.

The alteration of amino acids near the junction of the Fc portion and the non-Fc portion can dramatically increase the serum half-life of the Fc molecule (PCT publication WO 01/58957, the disclosure of which is hereby incorporated by reference). Accordingly, the junction region of a protein or polypeptide of the present invention can contain alterations that, relative to the naturally-occurring sequences of an immunoglobulin heavy chain and erythropoietin, preferably lie within about 10 amino acids of the junction point. These amino acid changes can cause an increase in hydrophobicity. In one embodiment, the constant region is derived from an IgG sequence in which the C-terminal lysine residue is replaced. Preferably, the C-terminal lysine of an IgG sequence is replaced with a non-lysine amino acid, such as alanine or leucine, to further increase serum half-life.

In certain embodiments, an antibody may be altered to increase, decrease or eliminate the extent to which it is glycosylated.

In one embodiment, the anti-hPD1 according to the invention has a heavy chain constant domain of SEQ ID NO. 39 or 52 and/or a light chain constant domain of SEQ ID. 40, particularly a heavy chain constant domain of SEQ ID NO. 39 or 52 and a light chain constant domain of SEQ ID. 40.

In another embodiment, the anti-hPD1 according to the invention has a heavy chain constant domain of SEQ ID NO: 52 and/or a light chain constant domain of SEQ ID. 40, particularly a heavy chain constant domain of SEQ ID NO:52 and a light chain constant domain of SEQ ID. 40.

TABLE E
Example of a heavy chain constant domain and a light chain constant domain
suitable for the humanized antibodies according to the invention.

| | |
|---|---|
| Heavy chain constant | ASTKGPSVFPLAPCSRSTSESTAALGCLVKDYFPEPVTVSWNSGALTSGVHTFPAVL |
| domain (IgG4m-S228P) | QSSGLYSLSSVVTVPSSSLGTKTYTCNVDHKPSNTKVDKRVESKYGPPCPPCPAPEFL |
| SEQ ID NO: 39 | GGPSVFLFPPKPKDTLMISRTPEVTCVVVDVSQEDPEVQFNWYVDGVEVHNAKTK |
| | PREEQFNSTYRVVSVLTVLHQDWLNGKEYKCKVSNKGLPSSIEKTISKAKGQPREPQ |
| | VYTLPPSQEEMTKNQVSLTCLVKGFYPSDIAVEWESNGQPENNYKTTPPVLDSDGS |
| | FFLYSRLTVDKSRWQEGNVFSCSVMHEALHNHYTQKSLSLSPGK |
| Light chain constant | RTVAAPSVFIFPPSDEQLKSGTASVVCLLNNFYPREAKVQWKVDNALQSGNSQESV |
| domain (CLkappa) | TEQDSKDSTYSLSSTLTLSKADYEKHKVYACEVTHQGLSSPVTKSFNRGEC |
| SEQ ID NO: 40 | |
| Heavy chain constant | ASTKGPSVFPLAPSSKSTSGGTAALGCLVKDYFPEPVTVSWNSGALTSGVHTFPAVL |
| domain (IgGlm- | QSSGLYSLSSVVTVPSSSLGTQTYICNVNHKPSNTKVDKKVEPKSCDKTHTCPPCPAP |
| N298A) | ELLGGPSVFLFPPKPKDTLMISRTPEVTCVVVDVSHEDPEVKFNWYVDGVEVHNAK |
| SEQ ID NO: 52 | TKPREEQYASTYRVVSVLTVLHQDWLNGKEYKCKVSNKALPAPIEKTISKAKGQPRE |
| | PQVYTLPPSRDELTKNQVSLTCLVKGFYPSDIAVEWESNGQPENNYKTTPPVLDSD |
| | GSFFLYSKLTVDKSRWQQGNVFSCSVMHEALHNHYTQKSLSLSPGK |

All subclass of Human IgG carries a C-terminal lysine residue of the antibody heavy chain (K444) that are cleaved off in circulation. This cleavage in the blood may compromise the bioactivity of the bifunctional molecule by releasing SIRPa. To circumvent this issue, K444 amino acid in the IgG1 or IgG4 domain may be substituted by an alanine to reduce proteolytic cleavage, a mutation commonly used for antibodies. Then, in one embodiment, the anti-PD1 antibody comprises at least one further amino acid substitution consisting of K444A.

In one embodiment, the anti-PD1 antibody comprises an additional cysteine residue at the C-terminal domain of the IgG to create an additional disulfide bond and potentially restrict the flexibility of the bifunctional molecule.

### Peptide Linker

This invention includes a bifunctional molecule which may comprise a peptide linker between the anti-PD-1 antibody or fragment thereof and SIRPa. The peptide linker usually has a length and flexibility enough to ensure that the two protein elements connected with the linker in between have enough freedom in space to exert their functions and avoid influences of the formation of a-helix and β-fold on the stability of the recombinant bifunctional molecule.

In an aspect of the disclosure, the anti-hPD1 antibody is preferably linked to SIRPa by a peptide linker. In other words, the invention relates to bifunctional molecule comprising an anti-PD1 antibody as detailed herein or an antigen binding fragment thereof, with a chain, e.g., the light or heavy chain or a fragment thereof, preferably the heavy chain or a fragment thereof, is linked to SIRPa through a peptide linker. As used herein, the term “linker” refers to a sequence of at least one amino acid that links SIRPa and the anti-PD-1 immunoglobulin sequence portion. Such a linker may be useful to prevent steric hindrances. The linker is usually 3-44 amino acid residues in length. Preferably, the linker has 3-30 amino acid residues. In some embodiments, the linker has 3, 4, 5, 6, 7, 8, 9, 10, 11, 12, 13, 14, 15, 16, 17, 18, 19, 20, 21, 22, 23, 24, 25, 26, 27, 28, 29 or 30 amino acid residues.

In an embodiment, the invention relates to a bifunctional molecule comprising an anti-PD-1 antibody or antigen-binding fragment thereof as defined above and SIRPa, wherein a chain of the antibody, e.g., the light or heavy chain, preferably the heavy chain, even more preferably the C-terminus of the heavy or light chain is linked to SIRPa, preferably to the N-terminus of SIRPa, by a peptide linker.

In a particular aspect, the invention relates to a bifunctional molecule comprising an anti-hPD-1 antibody or antigen-binding fragment thereof as defined above, wherein SIRPa is linked to the C-terminal end of the heavy chain of said antibody (e.g., the C-terminal end of the heavy chain constant domain), preferably by a peptide linker.

In an embodiment, the invention relates to bifunctional molecule comprising an anti-PD-1 antibody or antigen-binding fragment thereof as defined above, wherein SIRPa is linked to the C-terminal end of the light chain of said antibody (e.g., the C-terminal end of the light chain constant domain), preferably by a peptide linker.

The linker sequence may be a naturally occurring sequence or a non-naturally occurring sequence. If used for therapeutic purposes, the linker is preferably non-immunogenic in the subject to which the bifunctional molecule is administered. One useful group of linker sequences are linkers derived from the hinge region of heavy chain antibodies as described in WO 96/34103 and WO 94/04678. Other examples are poly-alanine linker sequences. Further preferred examples of linker sequences are Gly/Ser linkers of different length including (Gly4Ser)₄, (Gly4Ser)₃, (Gly4Ser)₂, Gly4Ser, Gly3Ser, Gly3, Gly2ser and (Gly3Ser2)₃, in particular (Gly4Ser)₃.

In one embodiment, the linker comprised in the bifunctional molecule is selected in the group consisting of (Gly4Ser)₄, (Gly4Ser)₃, (Gly4Ser)₂, Gly4Ser, Gly3Ser, Gly3, Gly2ser and (Gly3Ser2)₃, preferably is (Gly4Ser)₃.

In an embodiment, the invention relates to a bifunctional molecule that comprises an anti-PD-1 antibody or a fragment thereof as defined above wherein the antibody or a fragment thereof is linked to SIRPa by a linker sequence, preferably selected from the group consisting of (GGGGS)₃, (GGGGS)₄, (GGGGS)₂, GGGGS, GGGS, GGG, GGS and (GGGS)₃, even more preferably by (GGGGS)₃.

Preferably, the heavy chain, preferably the C terminus of the heavy chain of the anti-PD-1 antibody is genetically fused via a flexible (Gly4Ser)₃ linker to the N-terminus of SIRPa. At the fusion junction, the C-terminal lysine residue of the antibody heavy chain can be mutated to alanine to reduce proteolytic cleavage.

Preferably, the heavy chain, preferably the C terminus of the light chain of the anti-PD-1 antibody is genetically fused via a flexible (Gly4Ser)₃ linker to the N-terminus of SIRPa. At the fusion junction, the C-terminal lysine residue of the antibody light chain can be mutated to alanine to reduce proteolytic cleavage.

### SIRPa Molecule

The bifunctional molecule according to the invention comprises an additional or second entity that comprises a SIRPa molecule, a fragment or a variant thereof.

Preferably, the SIRPa protein is preferably a human SIRPa or fragments and variants thereof. In one embodiment, the bifunctional molecule comprises the typical wild-type SIRPa human protein of about 504 amino acids, preferably the extracellular domain of the wild-type human SIRPa protein (e.g. consisting of amino acids from positions 31 to 373 of the wild-type human SIRPa), optionally, with an additional N-terminal methionine residue (SEQ ID NO:51). Preferably the SIRPa protein is the protein of SEQ ID No: 51. The SIRPa is preferably devoid of its transmembrane domain and/or cytoplasmic domain. Preferably, the SIRPa protein consists of its extracellular domain, even more preferably consists essentially of the 31 to 373 amino acids of the wild-type human SIRPa.

A “variant” of a SIRPa protein is defined as an amino acid sequence that is altered by one or more amino acids. The variant can have “conservative” modifications or “non-conservative” modifications. Such modifications can include amino acid substitution, deletions and/or insertions. Guidance in determining which and how many amino acid residues may be substituted, inserted or deleted without abolishing biological properties (e.g. activity, binding capacity and/or structure) can be found using computer programs well known in the art, for example software for molecular modeling or for producing alignments. The variant SIRPa proteins included within the invention specifically include SIRPa proteins that retain substantially equivalent biological properties in comparison to a wild-type SIRPa. A variant of SIRPa also include altered polypeptides sequence of SIRPa (e.g. oxidized, reduced, deaminated or truncated forms). Particularly, truncations or fragment of SIRPa which retain comparable biological properties as the full-length SIRPa protein are included within the scope of the invention. In one embodiment, the SIRPa is any biological active fragment thereof. Variants of SIRPa include, more preferably, natural allelic variants resulting from natural polymorphism, including SNPs, splicing variants, etc.

The most common human SIRPa variants are SIRPa v1 and SIRPa v2 (accession number NP_542970 (P78324) and CAA71403). The SIRPa family may be divided into these two subsets; namely the SIRPa v1 isoform family and the SIRPa v2 isoform family. These families include the SIRPa Isoform 2 (identifier: P78324-2) and the SIRPa Isoform 4 (identifier: P78324-4), respectively. In one embodiment, the SIRPa variant is selected from the group consisting of the SIRPa isoform 2 (P78324-2) and the SIRPa isoform 4 (P78324-4).

Variant SIRPa proteins also include polypeptides that have at least about 55%, 60%, 65%, 70%, 75%, 80%, 85%, 90%, 92%, 95%, 96%, 97%, 98%, 99%, or more sequence identity with wild-type SIRPa. In one embodiment, the variant of SIRPalpha is SIRPgamma, which presents typically 55% sequence identity to wild-type SIRPa. As used herein, the terms “SIRP gamma”, “SIRPg” and “SIRPγ” are used interchangeably. For example, human SIRPg amino acid sequence is described in UniProtKB—Q9P1W8. SIRPγ has similar extracellular structure but different cytoplasmic regions giving contrasting types of signals. Indeed, SIRPalpha and SIRPgamma comprises 3 Ig-like extracellular domains: an Ig-like V-type, encoded by amino acids 32-137 (domain D1), an Ig-like C1-type 1 encoded by amino acids at positions 148-247 (domain D2), Ig-like C1-type 2 encoded by amino acids at positions 254-348 (domain D3). Then, in one embodiment, the SIRPa variant comprises i) D1 domain of SIRPg, D2 and D3 domains of SIRPa ii) D1 and D2 domains of SIRPg and D3 domain of SIRPa iii) D1 domain of SIRPg, D2 domain of SIRPa and D3 domain of SIRPg, iv) D1 domain of SIRPa, D2 and D3 domains of SIRPg, v) D1 and D2 domains of SIRPa and D3 domain of SIRPg or vi) D1 domain of SIRPa, D2 domain of SIRPg and D3 domain of SIRPa.

Preferred SIRPa according to the invention are human SIRPa polypeptides comprising or consisting of an amino acid sequence as described in SEQ ID No:51, US20160319256, WO 2013109752 or WO2016024021, as well as any natural variants and homologs thereof.

In some embodiments, the SIRP-α variant constructs have preferential activity at a diseased site (e.g., at the site of a tumor than at a non-diseased site). In some embodiments, the SIRP-α variants contain one or more substitutions of amino acids with histidine residues or with other amino acids that allow preferential binding of SIRP-α variant constructs at a diseased site.

In a particular embodiment, the SIRPa consists of truncations or fragment of the extracellular domain of SIRPa, specifically comprising or consisting of binding regions or of the amino acids within the set of contact residues that interact with CD47.

In one embodiment, the affinity of SIRPa protein can be measured using in vitro assays. Preferably, the SIRPa variants according to the invention maintain the affinity to CD47 of at least 10%, 20%, 30%, 40%, 50%, 60% in comparison with the wild type human SIRPa, preferably at least 80%, 90%, 95% and even more preferably 99% in comparison with the wild type SIRPa.

The invention also provides bifunctional molecules that comprises SIRPa proteins that have an enhanced affinity to CD47 compared to wild-type SIRPa proteins, for example, as described in WO 2013109752. In certain embodiments, the SIRP-α variant constructs have higher binding affinity to CD47 on diseased cells (e.g., tumor cells). In some embodiments, the SIRP-α variants bind with higher affinity to CD47 under acidic pH (e.g., less than around pH 7) and/or under hypoxic condition than under physiological conditions, for example as described in US 20160319256.

In one aspect, the SIRPa polypeptide used in the present invention is a recombinant SIRPa. The term “recombinant”, as used herein, means that the polypeptide is obtained or derived from a recombinant expression system, i.e., from a culture of host cells (e.g., microbial or insect or plant or mammalian) or from transgenic plants or animals engineered to contain a nucleic acid molecule encoding an SIRPa polypeptide. Preferably, the recombinant SIRPa is a human recombinant SIRPa, (e.g. a human SIRPa produced in recombinant expression system).

### Bifunctional Molecule or “Bicki”

The invention particularly provides a bifunctional molecule that comprises or consists in an anti-hPD1 antibody or antibody fragment thereof and SIRPa as disclosed hereabove, the anti-hPD1 antibody or antibody fragment thereof being covalently linked to SIRPa, preferably by a peptide linker as disclosed hereabove, particularly as a fusion protein.

Particularly, the bifunctional molecule according to the invention comprises two entities: a first entity comprising or consisting essentially of an anti-hPD1 antibody or fragment thereof; a second entity comprising or consisting essentially of SIRPa, preferably human SIRPa, even more preferably the extracellular domain of human SIRPa isoform 1, these two entities being optionally linked by a peptide linker.

Particularly, the bifunctional molecule according to the invention comprises one, two, three or four molecules of SIRPa. Particularly, the bifunctional molecule may comprise only one molecule of SIRPa, linked to only one light chain or heavy chain of the anti-PD-1 antibody. The bifunctional molecule may also comprise two molecules of SIRPa, linked to either the light or heavy chains of the anti-PD-1 antibody. The bifunctional molecule may also comprise two molecules of SIRPa, a first one linked to the light chain of the anti-PD-1 antibody and a second one linked to the heavy chain of the anti-PD-1 antibody. The bifunctional molecule may also comprise three molecules of SIRPa, two of them being linked to either the light or heavy chains of the anti-PD-1 antibody and the last one linked to the other chain of the anti-PD-1 antibody. Finally, the bifunctional molecule may also comprise four molecules of SIRPa, two molecules linked to the light chains of the anti-PD-1 antibody and two molecules linked to the heavy chains of the anti-PD-1 antibody. Accordingly, the bifunctional molecule comprises between one to four molecules of an immunotherapeutic agent as disclosed herein.

In one embodiment, only one of the light chains comprises one molecule of SIRPa (e.g. the bifunctional molecule comprises one molecule of SIRPa), only one of the heavy chains comprises one molecule of SIRPa (e.g. the bifunctional molecule comprises one molecule of SIRPa), each light chain comprises one molecule of SIRPa (e.g. the bifunctional molecule comprises two molecules of SIRPa), each heavy chain comprises one molecule of SIRPa (e.g. the bifunctional molecule comprises two molecules of SIRPa), only one of the light chain and only one of the heavy chain comprises one molecule of immunotherapeutic agent (e.g. the bifunctional molecule comprises two molecules of each light chain comprises one molecule of SIRPa and only one of the heavy chains comprises one molecule of SIRPa (e.g. the bifunctional molecule comprises three molecule of SIRPa), each heavy chain comprises one molecule of SIRPa and only one of the light chains comprises one molecule of SIRPa (e.g. the bifunctional molecule comprises three molecule of SIRPa), or both light chains and heavy chains comprises one molecule of SIRPa (e.g. the bifunctional molecule comprises four molecules of SIRPa).

In one embodiment, the bifunctional molecule according to the invention comprises or consists of:

(a) an anti-human PD-1 antibody or antigen-binding fragment thereof, which comprises (i) a heavy chain, and (ii) a light chain; and
(b) a human SIRPa or a fragment or variant thereof,
wherein the antibody heavy chain and/or light chain or a fragment thereof is covalently linked to SIRPa as a fusion protein, preferably by a peptide linker.

Preferably, the bifunctional molecule according to the invention comprises or consists of:

(a) a humanized anti-human PD-1 antibody or antigen-binding fragment thereof, which comprises (i) a heavy chain, and (ii) a light chain; and
(b) a human SIRPa or a fragment or variant thereof,
wherein the antibody heavy chain or light chain or a fragment thereof is covalently linked to SIRPa by a peptide linker.

Preferably, such bifunctional molecule comprises at least one peptide linker connecting the N-terminus of SIRPa to the C-terminus of the heavy chain or of the light chain or both of the anti-human PD-1 antibody, the peptide linker being preferably selected from the group consisting of (GGGGS)₃, (GGGGS)₄, (GGGGS)₂, GGGGS, GGGS, GGG, GGS and (GGGS)₃, even more preferably is (GGGGS)₃.

Preferably, the N-terminal end of SIRPa is connected to the C-terminal end of the heavy chain or of the light chain or both of the anti-human PD-1 antibody, though at least one peptide linker. Alternatively, the C-terminal end of SIRPa is connected to the N-terminal end of the heavy chain or of the light chain or both of the anti-human PD-1 antibody, though at least one peptide linker.

In one embodiment, the bifunctional molecule according to the invention comprises or consists of:

(a) an anti-human PD-1 antibody or antigen-binding fragment thereof, which comprises (i) a heavy chain, and (ii) a light chain,
(b) a human SIRPa or a fragment or variant thereof, and
(c) a peptide linker that connect the N-terminal end of SIRPa to the C-terminal end of the heavy chain or of the light chain or both of the anti-human PD-1 antibody, the peptide linker being preferably selected from the group consisting of (GGGGS)₃, (GGGGS)₄, (GGGGS)₂, GGGGS, GGGS, GGG, GGS and (GGGS)₃, even more preferably is (GGGGS)₃.

More specifically, the anti-human PD-1 or antigen binding fragment thereof can be any antibody as disclosed before in the section “Anti-PD-1” and the human SIRPa, fragment or variant thereof can be any SIPRa as disclosed before in the section “SIRPa molecule”.

In a particular embodiment, the bifunctional molecule according to the invention comprises or consists of:

(a) an anti-human PD-1 antibody or antigen-binding fragment thereof, which comprises:
(i) a heavy chain variable domain comprising HCDR1, HCDR2 and HCDR3, and
(ii) a light chain variable domain comprising LCDR1, LCDR2 and LCDR3,
   wherein:
the heavy chain CDR1 (HCDR1) comprises or consists of an amino acid sequence of SEQ ID NO: 1, optionally with one, two or three modification(s) selected from substitution(s), addition(s), deletion(s) and any combination thereof at any position but position 3 of SEQ ID NO: 1;
the heavy chain CDR2 (HCDR2) comprises or consists of an amino acid sequence of SEQ ID NO: 2, optionally with one, two or three modification(s) selected from substitution(s), addition(s), deletion(s) and any combination thereof at any position but positions 13, 14 and 16 of SEQ ID NO: 2;
the heavy chain CDR3 (HCDR3) comprises or consists of an amino acid sequence of SEQ ID NO: 3 wherein either X1 is D or E and X2 is selected from the group consisting of T, H, A, Y, N, E and S, preferably in the group consisting of H, A, Y, N and E; optionally with one, two or three modification(s) selected from substitution(s), addition(s), deletion(s) and any combination thereof at any position but positions 2, 3, 7 and 8 of SEQ ID NO: 3;
the light chain CDR1 (LCDR1) comprises or consists of an amino acid sequence of SEQ ID NO: 12 wherein X is G or T, optionally with one, two or three modification(s) selected from substitution(s), addition(s), deletion(s) and any combination thereof at any position but positions 5, 6, 10, 11 and 16 of SEQ ID NO: 12;
the light chain CDR2 (LCDR2) comprises or consists of an amino acid sequence of SEQ ID NO: 15, optionally with one, two or three modification(s) selected from substitution(s), addition(s), deletion(s) and any combination thereof; and
the light chain CDR3 (LCDR3) comprises or consists of an amino acid sequence of SEQ ID NO:16, optionally with one, two or three modification(s) selected from substitution(s), addition(s), deletion(s) and any combination thereof at any position but positions 1, 4 and 6 of SEQ ID NO: 16; and
   (b) a human SIRPa of SEQ ID No: 51 or a fragment or variant thereof, wherein the antibody heavy chain and/or light chain or a fragment thereof is covalently linked to SIRPa as a fusion protein, preferably by a peptide linker.

Preferably, the peptide linker is selected from the group consisting of (GGGGS)₃, (GGGGS)₄, (GGGGS)₂, GGGGS, GGGS, GGG, GGS and (GGGS)₃, even more preferably is (GGGGS)₃.

In another embodiment, the invention relates to a bifunctional molecule that comprises or consists of:

(a) a humanized anti-hPD1 antibody that comprises:
(i) a heavy chain variable region (VH) comprising or consisting of an amino acid sequence of SEQ ID NO: 17, wherein X1 is D or E and X2 is selected from the group consisting of T, H, A, Y, N, E and S preferably in the group consisting of H, A, Y, N, E; optionally with one, two or three modification(s) selected from substitution(s), addition(s), deletion(s) and any combination thereof at any position but positions 7, 16, 17, 20, 33, 38, 43, 46, 62, 63, 65, 69, 73, 76, 78, 80, 84, 85, 88, 93, 95, 96, 97, 98, 100, 101, 105, 106 and 112 of SEQ ID NO: 17;
(ii) a light chain variable region (VL) comprising or consisting of an amino acid sequence of SEQ ID NO: 26, wherein X is G or T, optionally with one, two or three modification(s) selected from substitution(s), addition(s), deletion(s) and any combination thereof at any position but positions 3, 4, 7, 14, 17, 18, 28, 29, 33, 34, 39, 42, 44, 50, 81, 88, 94, 97, 99 and 105 of SEQ ID NO: 26, and
(b) a human SIRPa of SEQ ID No: 51 or a variant thereof,
(c) a peptide linker selected from the group consisting of (GGGGS)₃, (GGGGS)₄, (GGGGS)₂, GGGGS, GGGS, GGG, GGS and (GGGS)₃, even more preferably is (GGGGS)₃, between the light chain and/or the heavy chain of the anti-hPD1 antibody and the human SIRPa or variant thereof.

Preferably, the N-terminal end of SIRPa is connected to the C-terminal end of the heavy chain or of the light chain or both of the anti-human PD-1 antibody, though at least one peptide linker. Alternatively, the C-terminal end of SIRPa is connected to the N-terminal end of the heavy chain or of the light chain or both of the anti-human PD-1 antibody, though at least one peptide linker.

In another embodiment, the invention relates to a bifunctional molecule that comprises or consists of:

(a) a humanized anti-hPD1 antibody that comprises:
(i) a heavy chain variable region (VH) comprising or consisting of an amino acid sequence of SEQ ID NO: 17, wherein X1 is D or E and X2 is selected from the group consisting of T, H, A, Y, N, E and S preferably in the group consisting of H, A, Y, N, E; optionally with one, two or three modification(s) selected from substitution(s), addition(s), deletion(s) and any combination thereof at any position but positions 7, 16, 17, 20, 33, 38, 43, 46, 62, 63, 65, 69, 73, 76, 78, 80, 84, 85, 88, 93, 95, 96, 97, 98, 100, 101, 105, 106 and 112 of SEQ ID NO: 17;
(ii) a light chain variable region (VL) comprising or consisting of an amino acid sequence of SEQ ID NO: 26, wherein X is G or T, optionally with one, two or three modification(s) selected from substitution(s), addition(s), deletion(s) and any combination thereof at any position but positions 3, 4, 7, 14, 17, 18, 28, 29, 33, 34, 39, 42, 44, 50, 81, 88, 94, 97, 99 and 105 of SEQ ID NO: 26, and
(b) a human SIRPa of SEQ ID No: 51 or a variant thereof,
wherein the C-terminal end of the heavy and/or light chain(s) of the antibody or antigen-binding fragment thereof is covalently linked to the N-terminal end of SIRPa to form a fusion protein, preferably by a (GGGGS)₃ peptide linker.

In a preferred embodiment, the C-terminal end of the heavy chain of the antibody or antigen-binding fragment thereof is covalently linked to the N-terminal end of SIRPa to form a fusion protein. Preferably, only the heavy chains of the antibody or antigen-binding fragment thereof are covalently linked to SIRPa. In a particular embodiment, the invention relates to a bifunctional molecule that comprises or consists of:

(a) a humanized anti-hPD1 antibody that comprises:
(i) a heavy chain variable region (VH) comprising or consisting of an amino acid sequence of SEQ ID NO: 17, wherein X1 is D or E and X2 is selected from the group consisting of T, H, A, Y, N, E and S preferably in the group consisting of H, A, Y, N, E; optionally with one, two or three modification(s) selected from substitution(s), addition(s), deletion(s) and any combination thereof at any position but positions 7, 16, 17, 20, 33, 38, 43, 46, 62, 63, 65, 69, 73, 76, 78, 80, 84, 85, 88, 93, 95, 96, 97, 98, 100, 101, 105, 106 and 112 of SEQ ID NO: 17;
(ii) a light chain variable region (VL) comprising or consisting of an amino acid sequence of SEQ ID NO: 26, wherein X is G or T, optionally with one, two or three modification(s) selected from substitution(s), addition(s), deletion(s) and any combination thereof at any position but positions 3, 4, 7, 14, 17, 18, 28, 29, 33, 34, 39, 42, 44, 50, 81, 88, 94, 97, 99 and 105 of SEQ ID NO: 26, and
(b) a human SIRPa of SEQ ID No: 51 or a variant thereof,
wherein the C-terminal end of the heavy chain of the antibody or antigen-binding fragment thereof is covalently linked to the N-terminal end of SIRPa to form a fusion protein, preferably by a (GGGGS)₃ peptide linker.

In another embodiment, the invention relates to a bifunctional molecule that comprises or consists of:

(a) a humanized anti-hPD1 antibody that comprises:
(i) a heavy chain variable region (VH) comprising or consisting of an amino acid sequence of SEQ ID NO: 18, 19, 20, 21, 22, 23, 24 or 25, optionally with one, two or three modification(s) selected from substitution(s), addition(s), deletion(s) and any combination thereof at any position but positions 7, 16, 17, 20, 33, 38, 43, 46, 62, 63, 65, 69, 73, 76, 78, 80, 84, 85, 88, 93, 95, 96, 97, 98, 100, 101, 105, 106 and 112 of SEQ ID NO: 18, 19, 20, 21, 22, 23, 24 or 25, respectively;
(ii) a light chain variable region (VL) comprising or consisting of an amino acid sequence of SEQ ID NO: 27 or SEQ ID NO: 28, optionally with one, two or three modification(s) selected from substitution(s), addition(s), deletion(s) and any combination thereof at any position positions 3, 4, 7, 14, 17, 18, 28, 29, 33, 34, 39, 42, 44, 50, 81, 88, 94, 97, 99 and 105 of SEQ ID NO: 27 or SEQ ID NO: 28.
(b) a human SIRPa of SEQ ID No: 51 or a variant thereof,
wherein the C-terminal end of the heavy and/or light chain(s) of the antibody or antigen-binding fragment thereof is covalently linked to the N-terminal end of SIRPa to form a fusion protein, preferably by a (GGGGS)₃ peptide linker.

Binding of the bifunctional molecules to their specific targets can be confirmed by, for example, enzyme-linked immunosorbent assay (ELISA), radioimmunoassay (RIA), FACS analysis, bioassay (e.g., growth inhibition), or Western Blot assay. Each of these assays generally detects the presence of protein-antibody complexes of particular interest by employing a labeled reagent (e.g., an antibody) specific for the complex of interest. For example, the anti-hPD-1 antibody/SIRPa complexes can be detected using e.g., an enzyme-linked antibody or antibody fragment which recognizes and specifically binds to SIRPa or to the receptor of SIRPa.

In some examples, the bifunctional molecule described herein suppresses the PD-1 signaling pathway by at least 20%, at least 40%, at least 50%, at least 75%, at least 90%, at least 100%, or by at least 2-fold, at least 5-fold, at least 10-fold, at least 20-fold, at least 50-fold, at least 100-fold, or at least 1000-fold.

Preferably, such bifunctional molecule has the ability to block or inhibit the interaction between PD-1 and its ligand (e.g. PD-L1 and/or PD-L2). In certain embodiments, the bifunctional molecule inhibits the binding interaction between PD-1 and its ligands (e.g. PD-L1 and/or PD-L2) by at least 50%. In certain embodiments, this inhibition may be greater than 60%, greater than 70%, greater than 80%, or greater than 90%.

In some examples, the bifunctional molecule described herein suppresses or decreases the PD-1 signaling pathway by at least 20%, at least 40%, at least 50%, at least 75%, at least 90%, at least 100%, or by at least 2-fold, at least 5-fold, at least 10-fold, at least 20-fold, at least 50-fold, at least 100-fold, or at least 1000-fold.

In some examples, the bifunctional molecule described herein stimulates IFN gamma secretion.

In some examples, the bifunctional molecule described herein blocks the interaction between CD47 expressing cells (e.g. tumor cells) and SIRPa expressing cells (e.g. antigen presenting cells (APC) such as macrophages).

In some examples, the bifunctional molecule described herein suppresses or decreases the SIRPa/CD47 signaling pathway by at least 10%, at least 20%, at least 40%, at least 50%, at least 75%, at least 90%, at least 100%, or by at least 2-fold, at least 5-fold, at least 10-fold, at least 20-fold, at least 50-fold, at least 100-fold, or at least 1000-fold.

In another example, the bifunctional molecule described herein potentiate the activation of T cells, stimulate the secretion of IFNg by T cells and/or stimulate proliferation of immune cells such as T cells.

In another example, the bifunctional molecule described herein induce cytokine secretion, and/or proliferation of naïve, partially exhausted T-cell subsets.

Preparation of Bifunctional Molecule—Nucleic Acid Molecules Encoding the Bifunctional Molecule, Recombinant Expression Vectors and Host Cells comprising such

To create a bifunctional molecule of the invention, an anti-hPD1 antibody of the invention is functionally linked to SIRPa.

Both entities of the bifunctional molecule are encoded in the same vector and produced as a fusion protein. Accordingly, also disclosed herein are nucleic acids encoding any of the bifunctional molecule described herein, vectors such as expression vectors or recombinant viruses comprising these nucleic acids, and host cells comprising the nucleic acids and/or vectors.

To produce a bifunctional fusion protein which is secreted in stable form by mammalian cells, according to the present invention, nucleic acid sequences coding for the bifunctional molecule are subcloned into an expression vector which is generally used to transfect mammalian cells. General techniques for producing molecules comprising antibody sequences are described in Coligan et al. (eds.), Current protocols in immunology, at pp. 10.19.1-10.19.11 (Wiley Interscience 1992), the contents of which are hereby incorporated by reference and in “Antibody engineering: a practical guide” from W. H. Freeman and Company (1992), in which commentary relevant to production of molecules is dispersed throughout the respective texts.

Generally, such method comprises the following steps of:

(1) transfecting or transforming appropriate host cells with the polynucleotide(s) or its variants encoding the recombinant bifunctional molecule of the invention or the vector containing the polynucleotide(s);
(2) culturing the host cells in an appropriate medium; and
(3) optionally isolating or purifying the protein from the medium or host cells.

The invention further relates to a nucleic acid encoding a bifunctional molecule as disclosed above, a vector, preferably an expression vector, comprising the nucleic acid of the invention, a genetically engineered host cell transformed with the vector of the invention or directly with the sequence encoding the recombinant bifunctional molecule, and a method for producing the protein of the invention by recombinant techniques.

The nucleic acid, the vector and the host cells are more particularly described hereafter.

### Nucleic Acid Sequence

The invention also relates to a nucleic acid molecule encoding the bifunctional molecule as defined above or to a group of nucleic acid molecules encoding the bifunctional molecule as defined above.

Antibody DNA sequences can for example be amplified from RNA of cells that synthesize an immunoglobulin, synthesized using PCR with cloned immunoglobulins, or synthesized via oligonucleotides that encode known signal peptide amino acid sequences. Preferably, the peptide signal comprises or consists of the amino acid sequence of SEQ ID NO: 49 for the VH and/or CH; and/or of the amino acid sequence of SEQ ID NO: 50 for the VL and/or CL. Particularly, the peptide signal is in the N-terminal of the CH, VH, CL and/or VL.

Such nucleic acid may encode an amino acid sequence comprising the VL and/or an amino acid sequence comprising the VH of the antibody (e.g., the light and/or heavy chains of the antibody). Such nucleic acid may be readily isolated and sequenced using conventional procedures.

Particularly, the nucleic acid molecules encoding the bifunctional molecule as defined above comprises:
a first nucleic acid molecule encoding a variable heavy chain domain of an anti-hPD-1 antibody as disclosed herein, optionally with a peptide signal of SEQ ID NO. 49, and
a second nucleic acid molecule encoding a variable light chain domain of an anti-hPD-1 antibody as disclosed herein, optionally with a peptide signal of SEQ ID NO. 50, and
a third nucleic acid encoding SIRPa or a variant thereof, preferably a human SIRPa, even more preferably the extracellular domain of the human SIRPa isoform 1 or a variant thereof, operably linked to either the first nucleic acid or to the second nucleic acid or both, optionally through a nucleic acid encoding a linker. In one embodiment, the nucleic acid molecules encoding the bifunctional molecule as defined above comprises:
a first nucleic acid molecule encoding a variable heavy chain domain of SEQ ID NO: 17, wherein X1 is D or E and X2 is selected from the group consisting of T, H, A, Y, N, E and S preferably in the group consisting of H, A, Y, N, and E; optionally with a peptide signal of SEQ ID NO. 49, and
a second nucleic acid molecule encoding a variable light chain domain of SEQ ID NO: 26, wherein X is G or T; optionally with a peptide signal of SEQ ID NO: 50, and
a third nucleic acid encoding human SIRPa of SEQ ID No:51 or a variant thereof operably linked to either the first nucleic acid or to the second nucleic acid or both, optionally through a nucleic acid encoding a linker.

Preferably, the nucleic acid molecules encoding the bifunctional molecule as defined above comprises:
a first nucleic acid molecule encoding a variable heavy chain domain of the amino acid sequence set forth in SEQ ID NO: 18, 19, 20, 21, 22, 23, 24 or 25; optionally with a peptide signal of SEQ ID NO. 49, and
a second nucleic acid molecule encoding a variable light chain domain of the amino acid sequence set forth in SEQ ID NO: 27 or SEQ ID NO: 28; optionally with a peptide signal of SEQ ID NO. 50, and
a third nucleic acid encoding human SIRPa of SEQ ID No:51 or a variant thereof operably linked to either the first nucleic acid or to the second nucleic acid or both, optionally through a nucleic acid encoding a peptide linker.

In a very particular embodiment, the nucleic acid molecule encoding a variable heavy chain domain has the sequence set forth in SEQ ID NO: 55 and/or the nucleic acid molecule encoding a variable light chain domain has the sequence set forth in SEQ ID NO: 56.

By operably linked is intended that the nucleic acid encodes a protein fusion including the variable heavy or light chain domain, optionally the peptide linker, and SIRPa. Preferably, the linker is selected from the group consisting of (GGGGS)₃, (GGGGS)₄, (GGGGS)₂, GGGGS, GGGS, GGG, GGS and (GGGS)₃, even more preferably is (GGGGS)₃.

In one embodiment, the nucleic acid molecule is an isolated, particularly non-natural, nucleic acid molecule.

The nucleic acid molecule or group of nucleic acid molecules encoding the bifunctional molecule according to the invention is(are) preferably comprised in a vector or a group of vectors.

### Vectors

In another aspect, the invention relates to a vector comprising the nucleic acid molecule or the group of nucleic acid molecules as defined above.

As used herein, a “vector” is a nucleic acid molecule used as a vehicle to transfer genetic material into a cell. The term “vector” encompasses plasmids, viruses, cosmids and artificial chromosomes. In general, engineered vectors comprise an origin of replication, a multicloning site and a selectable marker. The vector itself is generally a nucleotide sequence, commonly a DNA sequence, that comprises an insert (transgene) and a larger sequence that serves as the “backbone” of the vector. Modern vectors may encompass additional features besides the transgene insert and a backbone: promoter, genetic marker, antibiotic resistance, reporter gene, targeting sequence, protein purification tag. Vectors called expression vectors (expression constructs) specifically are for the expression of the transgene in the target cell, and generally have control sequences.

In one embodiment, both the heavy and light chain coding sequences and/or the constant region of the anti-PD1 antibody are included in one expression vector. Each of the heavy chain coding sequence and the light chain coding sequence may be in operable linkage to a suitable promoter, the heavy chain and/or the light chain being in operable linkage to an immunotherapeutic agent according to the invention. Alternatively, expression of both the heavy chain and the light chain may be driven by the same promoter. In another embodiment, each of the heavy and light chains of the antibody is cloned in to an individual vector, one or both of the heavy and light chains, the heavy chain and/or the light chain being in operable linkage to an immunotherapeutic agent according to the invention. In the latter case, the expression vectors encoding the heavy and light chains can be co-transfected into one host cell for expression of both chains, which can be assembled to form intact antibodies either in vivo or in vitro. Alternatively, the expression vector encoding the heavy chain and that encoding the light chain can be introduced into different host cells for expression each of the heavy and light chains, which can then be purified and assembled to form intact antibodies in vitro.

The nucleic acid molecule encoding the humanized anti-PD-1 antibody or antibody fragment thereof can be cloned into a vector by those skilled in the art, and then transformed into host cells. Accordingly, the present invention also provides a recombinant vector, which comprises a nucleic acid molecule encoding the anti-PD-1 antibody or fragment thereof of the present invention. In one preferred embodiment, the expression vector further comprises a promoter and a nucleic acid sequence encoding a secretion signal peptide, and optionally at least one drug-resistance gene for screening.

Suitable expression vectors typically contain (1) prokaryotic DNA elements coding for a bacterial replication origin and an antibiotic resistance marker to provide for the growth and selection of the expression vector in a bacterial host; (2) eukaryotic DNA elements that control initiation of transcription, such as a promoter; and (3) DNA elements that control the processing of transcripts, such as a transcription termination/polyadenylation sequence.

The methods known to the artisans in the art can be used to construct an expression vector containing the nucleic acid sequence of the bifunctional molecule described herein and appropriate regulatory components for transcription/translation. These methods include in vitro recombinant DNA techniques, DNA synthesis techniques, in vivo recombinant techniques, etc. The DNA sequence is efficiently linked to a proper promoter in the expression vector to direct the synthesis of mRNA. The expression vector may further comprise a ribosome-binding site for initiating the translation, transcription terminator and the like.

An expression vector can be introduced into host cells using a variety of techniques including calcium phosphate transfection, liposome-mediated transfection, electroporation, and the like. Preferably, transfected cells are selected and propagated wherein the expression vector is stably integrated in the host cell genome to produce stable transformants. Techniques for introducing vectors into eukaryotic cells and techniques for selecting stable transformants using a dominant selectable marker are described by Sambrook, by Ausubel, by Bebbington, “Expression of Antibody Genes in Nonlymphoid Mammalian Cells,” in 2 METHODS: A companion to methods in enzymology 136 (1991), and by Murray (ed.), Gene transfer and expression protocols (Humana Press 1991). Suitable cloning vectors are described by Sambrook et al. (eds.), MOLECULAR CLONING: A LABORATORY MANUAL, Second Edition (Cold Spring Harbor Press 1989) (hereafter “Sambrook”); by Ausubel et al. (eds.), CURRENT PROTOCOLS IN MOLECULAR BIOLOGY (Wiley Interscience 1987) (hereafter “Ausubel”); and by Brown (ed.), MOLECULAR BIOLOGY LABFAX (Academic Press 1991).

### Host Cells

In another aspect, the invention relates to a host cell comprising a vector or a nucleic acid molecule or group of nucleic acid molecules as defined above, for example for bifunctional molecule production purposes.

As used herein, the term “host cell” is intended to include any individual cell or cell culture that can be or has been recipient of vectors, exogenous nucleic acid molecules, and polynucleotides encoding the antibody construct of the present invention; and/or recipients of the antibody construct or bifunctional molecule itself. The introduction of the respective material into the cell can be carried out by way of transformation, transfection and the like. The term “host cell” is also intended to include progeny or potential progeny of a single cell. Suitable host cells include prokaryotic or eukaryotic cells, and also include but are not limited to bacteria, yeast cells, fungi cells, plant cells, and animal cells such as insect cells and mammalian cells, e.g., murine, rat, rabbit, macaque or human.

In one embodiment, a host cell comprises (e.g., has been transformed with): (1) a vector comprising a nucleic acid that encodes an amino acid sequence comprising the VL of the antibody and/or an amino acid sequence comprising the VH of the antibody and/or the constant region of the antibody, or (2) a first vector comprising a nucleic acid that encodes an amino acid sequence comprising the VL of the antibody and a second vector comprising a nucleic acid that encodes an amino acid sequence comprising the VH of the antibody.

In another embodiment, a host cell comprises (e.g., has been transformed with) a vector comprising both of the entities of the bifunctional molecule. Preferably, a host cell comprises (e.g., has been transformed with) a vector comprising a first nucleic acid molecule encoding a variable heavy chain domain of an anti-hPD-1 antibody as disclosed herein, and a second nucleic acid molecule encoding a variable light chain domain of an anti-hPD-1 antibody as disclosed herein, operably linked to a third nucleic acid encoding SIRPa or a variant thereof.

A method of humanized anti-PD1 antibody production is also provided herein. The method comprises culturing a host cell comprising a nucleic acid encoding the antibody, as provided above, under conditions suitable for expression of the antibody, and optionally recovering the antibody from the host cell (or host cell culture medium). Particularly, for recombinant production of a humanized anti-PD1 antibody, nucleic acid encoding an antibody, e.g., as described above, is isolated and inserted into one or more vectors for further cloning and/or expression in a host cell.

A bifunctional molecule of the present invention is preferably expressed in eukaryotic cells such as mammalian cells, plant cells, insect cells or yeast cells. Mammalian cells are especially preferred eukaryotic hosts because mammalian cells provide suitable post-translational modifications such as glycosylation. Preferably, such suitable eukaryotic host cell may be fungi such as Pichia pastoris, Saccharomyces cerevisiae, Schizosaccharomyces pombe; insect cell such as Mythimna separate; plant cell such as tobacco, and mammalian cells such as BHK cells, 293 cells, CHO cells, NSO cells and COS cells. Other examples of useful mammalian host cell lines are CV-1 in Origin with SV40 genes cell (COS cell), monkey kidney CV1 line transformed by SV40 (COS-7); human embryonic kidney line (293 or 293 cells as described, e.g., in Graham, F. L. et al, J. Gen Virol. 36 (1977) 59-74); baby hamster kidney cells (BHK); mouse Sertoli cells (TM4 cells as described, e.g., in Mather, J. P., Biol. Reprod. 23 (1980) 243-252); Human Epithelial Kidney cell (HEK cell); monkey kidney cells (CV1); African green monkey kidney cells (VERO-76); human cervical carcinoma cells (HELA); canine kidney cells (MDCK; buffalo rat liver cells (BRL 3 A); human lung cells (W138); human liver cells (Hep G2); mouse mammary tumor (MMT 060562); TRI cells, as described, e.g., in Mather, J. P. et al, Annals N.Y. Acad. Sci. 383 (1982) 44-68; MRC 5 cells; and FS4 cells. Other useful mammalian host cell lines include Chinese hamster ovary (CHO) cells, including DHFR″ CHO cells (Urlaub, G. et al, Proc. Natl. Acad. Sci. USA 77 (1980) 4216-4220); and myeloma cell lines such as YO, NSO and Sp2/0. For a review of certain mammalian host cell lines suitable for antibody production, see, e.g., Yazaki, P. and Wu, A. M., Methods in Molecular Biology, Vol. 248, Lo, B. K. C. (ed.), Humana Press, Totowa, N.J. (2004), pp. 255-268. For example, mammalian cell lines that are adapted to grow in suspension may be useful.

Particularly, the host cell of the present invention is selected from the group consisting of CHO cell, COS cell, NSO cell, and HEK cell.

For a mammalian host, the transcriptional and translational regulatory signals of the expression vector may be derived from viral sources, such as adenovirus, bovine papilloma virus, simian virus, or the like, in which the regulatory signals are associated with a particular gene which has a high level of expression. Suitable transcriptional and translational regulatory sequences also can be obtained from mammalian genes, such as actin, collagen, myosin, and metallothionein genes.

Stable transformants that produce a bifunctional molecule according to the invention can be identified using a variety of methods. After molecule-producing cells have been identified, the host cells are cultured under conditions (e.g. temperature, medium) suitable for their growth and for bifunctional molecule expression. The bifunctional molecules are then isolated and/or purified by any methods known in the art. These methods include, but are not limited to, conventional renaturation treatment, treatment by protein precipitant (such as salt precipitation), centrifugation, cell lysis by osmosis, sonication, supercentrifugation, molecular sieve chromatography or gel chromatography, adsorption chromatography, ion exchange chromatography, HPLC, any other liquid chromatography, and the combination thereof. As described, for example, by Coligan, bifunctional molecule isolation techniques may particularly include affinity chromatography with Protein-A Sepharose, size-exclusion chromatography and ion exchange chromatography. Protein A preferably is used to isolate the bifunctional molecules of the invention.

### Pharmaceutical Composition and Method of Administration Thereof

The present invention also relates to a pharmaceutical composition comprising any of the bifunctional molecule described herein, the nucleic acid molecule, the group of nucleic acid molecules, the vector and/or the host cells as described hereabove, preferably as the active ingredient or compound. The formulations can be sterilized and, if desired, mixed with auxiliary agents such as pharmaceutically acceptable carriers and excipients which do not deleteriously interact with the bifunctional molecule, nucleic acid, vector and/or host cell of the invention. Optionally, the pharmaceutical composition may further comprise an additional therapeutic agent as detailed below.

Preferably, the pharmaceutical compositions of the present invention may comprise a bifunctional molecule as described herein, the nucleic acid molecule, the group of nucleic acid molecules, the vector and/or the host cells as described hereabove in combination with one or more pharmaceutically or physiologically acceptable carriers, diluents, excipients, salt, and anti-oxidant as described hereafter. Desirably, a pharmaceutically acceptable form is employed which does not adversely affect the desired immune potentiating effects of the bifunctional molecule according to the invention. To facilitate administration, the bifunctional molecule as described herein can be made into a pharmaceutical composition for in vivo administration. The means of making such a composition have been described in the art (see, for instance, Remington: The Science and Practice of Pharmacy, Lippincott Williams & Wilkins, 21st edition (2005).

Particularly, the pharmaceutical composition according to the invention can be formulated for any conventional route of administration including a topical, enteral, oral, parenteral, intranasal, intravenous, intramuscular, subcutaneous or intraocular administration and the like. Preferably, the pharmaceutical composition according to the invention is formulated for enteral or parenteral route of administration. Compositions and formulations for parenteral administration may include sterile aqueous solutions that may also contain buffers, diluents and other suitable additives such as, but not limited to, penetration enhancers, carder compounds and other pharmaceutically acceptable carriers or excipients.

The pharmaceutical composition may be prepared by mixing an agent having the desired degree of purity with optional pharmaceutically acceptable carriers, excipients or stabilizers (Remington's Pharmaceutical Sciences 16th edition, Osol, A. Ed. (1980)), in the form of lyophilized formulations or aqueous solutions. Acceptable carriers, excipients, or stabilizers are nontoxic to recipients at the dosages and concentrations employed, and include buffers such as phosphate, citrate, and other organic acids; antioxidants including ascorbic acid and methionine; preservatives (such as octadecyldimethylbenzyl ammonium chloride; hexamethonium chloride; benzalkonium chloride, benzethonium chloride; phenol, butyl or benzyl alcohol; alkyl parabens such as methyl or propyl paraben; catechol; resorcinol; cyclohexanol; 3-pentanol; and m-cresol); low molecular weight (less than about 10 residues) polypeptides; proteins, such as serum albumin, gelatin, or immunoglobulins; hydrophilic polymers such as polyvinylpyrrolidone; amino acids such as glycine, glutamine, asparagine, histidine, arginine, or lysine; monosaccharides, disaccharides, and other carbohydrates including glucose, mannose, or dextrins; chelating agents such as EDTA; sugars such as sucrose, mannitol, trehalose or sorbitol; salt-forming counter-ions such as sodium; metal complexes (e.g., Zn-protein complexes); and/or non-ionic surfactants such as TWEEN™, PLURONICS™ or polyethylene glycol (PEG).

A solid pharmaceutically acceptable vehicle may include one or more substances which may also act as flavoring agents, lubricants, solubilizers, suspending agents, dyes, fillers, glidants, compression aids, inert binders, sweeteners, preservatives, dyes, coatings, or tablet-disintegrating agents. Suitable solid vehicles include, for example calcium phosphate, magnesium stearate, talc, sugars, lactose, dextrin, starch, gelatin, cellulose, polyvinylpyrrolidine, low melting waxes and ion exchange resins. Pharmaceutically acceptable carriers include sterile aqueous solutions or dispersions and sterile powders for the extemporaneous preparation of sterile injectable solutions or dispersion. Except insofar as any conventional media or agent is incompatible with the active compound, use thereof in the pharmaceutical compositions of the invention is contemplated.

The bifunctional molecule according to the invention may be dissolved or suspended in a pharmaceutically acceptable liquid vehicle such as water, an organic solvent, ethanol, polyol (for example, glycerol, propylene glycol, and liquid polyethylene glycol, and the like a mixture of both or pharmaceutically acceptable oils or fats and suitable mixtures thereof. The liquid vehicle can contain other suitable pharmaceutical additives such as solubilizers, emulsifiers, buffers, preservatives, sweeteners, flavoring agents, suspending agents, wetting agents, thickening agents, colors, viscosity regulators, stabilizers or osmo-regulators. Suitable examples of liquid vehicles for oral and enteral administration include water (partially containing additives as above, e.g. cellulose derivatives, preferably sodium carboxymethyl cellulose solution), alcohols (including monohydric alcohols and polyhydric alcohols, e.g. glycols) and their derivatives, and oils (e.g. fractionated coconut oil and peanut oil). For parenteral administration, the vehicle can also be an oily ester such as ethyl oleate and isopropyl myristate. Sterile liquid vehicles are useful in sterile liquid form compositions for enteral administration. The liquid vehicle for pressurized compositions can be a halogenated hydrocarbon or other pharmaceutically acceptable propellant.

The pharmaceutical composition of the invention may further comprise one or more pharmaceutically acceptable salts. A “pharmaceutically acceptable salt” refers to a salt that retains the desired biological activity of the parent compound and does not impart any undesired toxicological effects. Examples of such salts include acid addition salts and base addition salts. Acid addition salts include those derived from nontoxic inorganic acids, such as hydrochloric, nitric, phosphoric, sulfuric, hydrobromic, hydroiodic, phosphorous and the like, as well as from nontoxic organic acids such as aliphatic mono- and dicarboxylic acids, phenyl-substituted alkanoic acids, hydroxy alkanoic acids, aromatic acids, aliphatic and aromatic sulfonic acids and the like. Base addition salts include those derived from alkaline metals or alkaline earth metals, such as sodium, potassium, magnesium, calcium and the like, as well as from nontoxic organic amines, such as N,N′-dibenzylethylenediamine, N-methylglucamine, chloroprocaine, choline, diethanolamine, ethylenediamine, procaine and the like.

A pharmaceutical composition of the invention also may include a pharmaceutically acceptable anti-oxidant. Examples of pharmaceutically acceptable antioxidants include: water soluble antioxidants, such as ascorbic acid, cysteine hydrochloride, sodium bisulfate, sodium metabisulfite, sodium sulfite and the like; oil-soluble antioxidants, such as ascorbyl palmitate, butylated hydroxyanisole (BHA), butylated hydroxytoluene (BHT), lecithin, propyl gallate, alpha-tocopherol, and the like; and metal chelating agents, such as citric acid, ethylenediamine tetra-acetic acid (EDTA), sorbitol, tartaric acid, phosphoric acid, and the like.

To facilitate delivery, any of the bifunctional molecule or its encoding nucleic acids can be conjugated with a chaperon agent. The chaperon agent can be a naturally occurring substance, such as a protein (e.g., human serum albumin, low-density lipoprotein, or globulin), carbohydrate (e.g., a dextran, pullulan, chitin, chitosan, inulin, cyclodextrin or hyaluronic acid), or lipid. It can also be a recombinant or synthetic molecule, such as a synthetic polymer, e.g., a synthetic polyamino acid. Examples of polyamino acids include polylysine (PLL), poly L aspartic acid, poly L-glutamic acid, styrene-maleic acid anhydride copolymer, poly(L-lactide-co-glycolied) copolymer, divinyl ether-maleic anhydride copolymer, N-(2-hydroxypropyl) methacrylamide copolymer (HMPA), polyethylene glycol (PEG), polyvinyl alcohol (PVA), polyurethane, poly(2-ethylacryllic acid), N-isopropylacrylamide polymers, and polyphosphazine. In one example, the chaperon agent is a micelle, liposome, nanoparticle, or microsphere. Methods for preparing such a micelle, liposome, nanoparticle, or microsphere are well known in the art. See, e.g., U.S. Pat. Nos. 5,108,921; 5,354,844; 5,416,016; and 5,527,5285.

Pharmaceutical composition typically must be sterile and stable under the conditions of manufacture and storage. The pharmaceutical composition can be formulated as a solution, micro-emulsion, liposome, or other ordered structure suitable to high drug concentration and/or in suitable for injection. The proper fluidity can be maintained, for example, by the use of a coating such as lecithin, by the maintenance of the required particle size in the case of dispersion and by the use of surfactants.

In one embodiment, the pharmaceutical composition is an injectable composition that may contain various carriers such as vegetable oils, dimethylactamide, dimethyformamide, ethyl lactate, ethyl carbonate, isopropyl myristate, ethanol, and polyols (glycerol, propylene glycol, liquid polyethylene glycol, and the like). For intravenous injection, water soluble antibodies can be administered by the drip method, whereby a pharmaceutical formulation containing the antibody and physiologically acceptable excipients is infused. Physiologically acceptable excipients may include, for example, 5% dextrose, 0.9% saline, Ringer's solution or other suitable excipients. Intramuscular preparations, e.g., a sterile formulation of a suitable soluble salt form of the antibody, can be dissolved and administered in a pharmaceutical excipient such as Water-for-Injection, 0.9% saline, or 5% glucose solution.

Sterile injectable solutions can be prepared by incorporating the active compound in the required amount in an appropriate solvent with one or a combination of ingredients enumerated above, as required, followed by sterilization microfiltration. Generally, dispersions are prepared by incorporating the active compound into a sterile vehicle that contains a basic dispersion medium and the required other ingredients from those enumerated above. In the case of sterile powders for the preparation of sterile injectable solutions, the preferred methods of preparation are vacuum drying and freeze-drying (lyophilization) that yield a powder of the active ingredient plus any additional desired ingredient from a previously sterile-filtered solution thereof. Prolonged absorption of the injectable compositions can be brought about by including in the composition an agent that delays absorption, for example, monostearate salts and gelatin.

Prevention of presence of microorganisms may be ensured both by sterilization procedures, and by the inclusion of various antibacterial and antifungal agents, for example, chlorobutanol, phenol sorbic acid, and the like. It may also be desirable to include isotonic agents, such as sugars, sodium chloride, and the like into the compositions. In addition, prolonged absorption of the injectable pharmaceutical form may be brought about by the inclusion of agents which delay absorption such as aluminum monostearate and gelatin.

It will be understood by one skilled in the art that the formulations of the invention may be isotonic with human blood that is the formulations of the invention have essentially the same osmotic pressure as human blood. Such isotonic formulations generally have an osmotic pressure from about 250 mOSm to about 350 mOSm. Isotonicity can be measured by, for example, a vapor pressure or ice-freezing type osmometer. Tonicity of a formulation is adjusted by the use of tonicity modifiers. “Tonicity modifiers” are those pharmaceutically acceptable inert substances that can be added to the formulation to provide an isotonicity of the formulation. Tonicity modifiers suitable for this invention include, but are not limited to, saccharides, salts and amino acids.

Pharmaceutical compositions according to the invention may be formulated to release the active ingredients (e.g. the bifunctional molecule of the invention) substantially immediately upon administration or at any predetermined time or time period after administration. The pharmaceutical composition in some aspects can employ time-released, delayed release, and sustained release delivery systems such that the delivery of the composition occurs prior to, and with sufficient time to cause, sensitization of the site to be treated. Means known in the art can be used to prevent or minimize release and absorption of the composition until it reaches the target tissue or organ, or to ensure timed-release of the composition. Such systems can avoid repeated administrations of the composition, thereby increasing convenience to the subject and the physician.

The amount of active ingredient which can be combined with a carrier material to produce a single dosage form will vary depending upon the subject being treated, and the particular mode of administration. The amount of active ingredient which can be combined with a carrier material to produce a single dosage form will generally be that amount of the composition which produces a therapeutic effect.

### Subject, Regimen and Administration

The present invention relates to a bifunctional molecule as disclosed herein; a nucleic acid or a vector encoding such, a host cell or a pharmaceutical composition, a nucleic acid, a vector or a host cell, for use as a medicament or for use in the treatment of a disease or for administration in a subject or for use as a medicament. Examples of treatments are more particularly described hereafter under the section “Methods and Uses”. It also relates to the use of a pharmaceutical composition, a nucleic acid, a vector or a host cell of the present invention or a bifunctional molecule comprising an anti-PD1 antibody or antibody fragment thereof and SIRPa in the manufacture of a medicament for treating a disease in a subject. Finally, it relates to a method for treating a disease or a disorder in a subject comprising administering a therapeutically effective amount of a pharmaceutical composition or a bifunctional molecule comprising an anti-PD1 antibody or antibody fragment thereof and SIRPa to the subject. Examples of treatments are more particularly described hereafter under the section “Methods and Uses”. The subject to treat may be a human, particularly a human at the prenatal stage, a new-born, a child, an infant, an adolescent or an adult, in particular an adult of at least 30 years old, 40 years old, preferably an adult of at least 50 years old, still more preferably an adult of at least 60 years old, even more preferably an adult of at least 70 years old.

Particularly, the subject is affected with a disease that may involve the PD-1/PDL-1 pathway, particularly wherein, at least one of the ligands of PD-1 (e.g. PDL-1 and/or PDL-2) or PD-1 is/are expressed, especially overexpressed. Preferably, the subject is suffering from cancer, even more preferably from a PD1, PD-L1 and/or PD-L2 positive cancer or a PD-1 positive cancer. Examples of diseases and cancers are more particularly described hereafter under the section “Methods and Uses”.

In a particular embodiment, the subject has already received at least one line of treatment, preferably several lines of treatment, prior to the administration of a bifunctional molecule comprising an anti-PD1 antibody or antibody fragment thereof and SIRPa according to the invention or of a pharmaceutical composition according to the invention.

Conventional methods, known to those of ordinary skill in the art of medicine, can be used to administer bifunctional molecule or the pharmaceutical composition disclosed herein to the subject, depending upon the type of diseases to be treated or the site of the disease. This composition can be administered via conventional routes, e.g., administered orally, parenterally, enterally, by inhalation spray, topically, rectally, nasally, buccally, vaginally or via an implanted reservoir. The term “parenterally” as used herein includes subcutaneous, intra-cutaneous, intravenous, intramuscular, intra-articular, intra-arterial, intra-synovial, intra-tumoral, intra-sternal, intra-thecal, intra-lesion, and intracranial injection or infusion techniques. When administered parenterally, the pharmaceutical composition according to the invention is preferably administered by intravenous route of administration. When administered enterally, the pharmaceutical composition according to the invention is preferably administered by oral route of administration. This composition can also be administered locally.

The form of the pharmaceutical compositions, the route of administration and the dose of administration of the pharmaceutical composition or the bifunctional molecule according to the invention can be adjusted by the man skilled in the art according to the type and severity of the infection, and to the patient, in particular its age, weight, sex, and general physical condition. The compositions of the present invention may be administered in a number of ways depending upon whether local or systemic treatment is desired.

Preferably, the treatment with the bifunctional molecule or with a pharmaceutical composition according to the invention is administered regularly, preferably between every day, every week or every month, more preferably between every day and every one, two, three or four weeks. In a particular embodiment, the treatment is administered several times a day, preferably 2 or 3 times a day.

The duration of treatment with the bifunctional molecule or with a pharmaceutical composition according to the invention according to the invention is preferably comprised between 1 day and 20 weeks, more preferably between 1 day and 10 weeks, still more preferably between 1 day and 4 weeks, even more preferably between 1 day and 2 weeks. Alternatively, the treatment may last as long as the disease persists.

The bifunctional molecule disclosed herein may be provided at an effective dose range from about 1 ng/kg body weight to about 30 mg/kg body weight, 1 μg/kg to about 20 mg/kg, 10 μg/kg to about 10 mg/kg, or from 100 μg/kg to 5 mg/kg, optionally every one, two, three or four weeks, preferably by parenteral or oral administration, in particular by intravenous or subcutaneous administration.

Typically, the bifunctional molecule disclosed herein may be provided at an effective dose range from about 1 mg/kg body weight to about 20 mg/kg body weight, advantageously 2 to 10 mg/kg, and in particular 3, 4, 5, 6, 7 mg/kg which is appropriate for antibodies safe administration and very satisfying for the clinical need.

Particularly, the bifunctional molecule according to the invention can be administered at a subtherapeutic dose. The term “subtherapeutic dose” as used herein refers to a dose that is below the effective monotherapy dosage levels commonly used to treat a disease, or a dose that currently is not typically used for effective monotherapy with anti-hPD1 antibodies.

### Methods and Uses

### Use in the Treatment of a Disease

The bifunctional molecules, nucleic acids, vectors, host cells, compositions and methods of the present invention have numerous in vitro and in vivo utilities and applications. For example, the bifunctional molecule, the nucleic acids, the vectors, the host cells and/or the pharmaceutical compositions described herein can be used as therapeutic agents, diagnostic agents and medical researches. Particularly, any of the bifunctional molecule, nucleic acid molecule, group of nucleic acid molecules, vector, host cells or pharmaceutical composition provided herein may be used in therapeutic methods and/or for therapeutic purposes. Particularly, the bifunctional molecule, nucleic acid, vector or pharmaceutical composition provided herein may be useful for the treatment of any disease or condition, preferably involving PD-1, such as cancer, autoimmune disease, and infection or other diseases associated with immune deficiency, such as T cell dysfunction. Even more preferably, the invention relates to a method of treatment of a disease and/or disorder selected from the group consisting of a cancer, an infectious disease and a chronic viral infection in a subject in need thereof comprising administering to said subject an effective amount of the bifunctional molecule or pharmaceutical composition as defined above. Examples of such diseases are more particularly described hereafter.

Particularly, the bifunctional molecule according to the invention are called “bifunctional checkpoint inhibitors” as they target both PD-1/PD-L1/PD-L2 and SIRPa pathways.

The invention particularly concerns a bifunctional molecule, a nucleic acid, a group of nucleic acids or a vector encoding such, or a pharmaceutical composition comprising such for use in the treatment of a pathology, disease and/or disorder that could be prevented or treated by the inhibition of the binding of PD-L1 and/or PD-L2 to PD-1; and/or by the inhibition of the binding of CD47 to SIRPa.

Accordingly, disclosed herein are methods for treating a disease, in particular associated with the PD-1 and/or PD-1/PD-L1 and/or PD-1/PD-L2 signaling pathway, comprising administering to a subject in need of a treatment an effective amount of any of the bifunctional molecule or pharmaceutical composition described herein. Physiological data of the patient (e.g. age, size, and weight) and the routes of administration have also to be taken into account to determine the appropriate dosage, so as a therapeutically effective amount will be administered to the patient.

Disclosed herein are additionally or alternatively methods for treating a disease, in particular associated with the SIRPa/CD47 pathway, comprising administering to a subject in need of a treatment an effective amount of any of the bifunctional molecule or pharmaceutical composition described herein.

In another aspect the bifunctional molecules disclosed herein can be administered to a subject, e.g., in vivo, to enhance immunity, preferably in order to treat a disorder and/or disease. Accordingly, in one aspect, the invention provides a method of modifying an immune response in a subject comprising administering to the subject a bifunctional molecule, nucleic acid, vector or pharmaceutical composition of the invention such that the immune response in the subject is modified. Preferably, the immune response is enhanced, increased, stimulated or up-regulated. The bifunctional molecule or pharmaceutical composition can be used to enhance immune responses such as T cell activation in a subject in need of a treatment. The immune response enhancement can result in the inhibition of the binding of PD-L1 and/or PD-L2 to PD-1 and/or CD47 to SIRPa, thereby reducing the immunosuppressive environment, stimulating the proliferation and/or the activation of human T-cells and/or the IFNγ secretion by human PBMC.

The invention particularly provides a method of enhancing an immune response in a subject, comprising administering to the subject a therapeutic effective amount of any of the bifunctional molecule, nucleic acid, vector or pharmaceutical composition comprising such described herein, such that an immune response in the subject is enhanced.

In some embodiments, the amount of the bifunctional molecule described herein is effective in suppressing the PD-1 signaling (e.g., reducing the PD-1 signaling by at least 20%, 30%, 50%, 80%, 100%, 200%, 400%, or 500% as compared to a control). In other embodiments, the amount of the bifunctional molecule described herein is effective in activating immune responses (e.g., by at least 20%, 30%, 50%, 80%, 100%, 200%, 400%, or 500% as compared to a control).

In some embodiments, the amount of the bifunctional molecule described herein is effective in the inhibition of the binding of human PD-L1 and/or PD-L2 to human PD-1 e.g., inhibiting the binding by at least 20%, 30%, 50%, 80%, 100%, 200%, 400%, or 500% as compared to a control.

In some embodiments, the amount of the bifunctional molecule described herein is sufficient to have an antagonist activity of the binding of human PD-L1 and/or PD-L2 to human PD-1 e.g., inhibiting the binding by at least 20%, 30%, 50%, 80%, 100%, 200%, 400%, or 500% as compared to a control.

In some embodiments, the amount of the bifunctional molecule described herein is effective in suppressing the SIRPa/CD47 signaling (e.g., reducing the SIRPa/CD47 signaling by at least 20%, 30%, 50%, 80%, 100%, 200%, 400%, or 500% as compared to a control). In other embodiments, the amount of the bifunctional molecule described herein is effective in activating immune responses (e.g., by at least 20%, 30%, 50%, 80%, 100%, 200%, 400%, or 500% as compared to a control).

In some embodiments, the amount of the bifunctional molecule described herein is effective in the inhibition of the binding of CD47 expressed by tumoral cells to human SIRPa expressed by APC e.g., inhibiting the binding by at least 20%, 30%, 50%, 80%, 100%, 200%, 400%, or 500% as compared to a control condition (i.e. without the bifunctional molecule of the invention).

In some embodiments, the amount of the bifunctional molecule described herein is sufficient to have a competitive activity for the binding of CD47, particularly a competition with human immune cells expressing SIRPa such as macrophages, e.g., inhibiting the binding between CD47 and SIRPa positive cells by at least 20%, 30%, 50%, 80%, 100%, 200%, 400%, or 500% as compared to a control condition (i.e. without the bifunctional molecule of the invention).

The present invention also relates to a bifunctional molecule as described herein; a nucleic acid or a vector encoding such, or a pharmaceutical composition comprising such for use in the treatment of a disorder and/or disease in a subject and/or for use as a medicament or vaccine. It also relates to the use of a bifunctional molecule as described herein; a nucleic acid or a vector encoding such, or a pharmaceutical composition comprising such in the manufacture of a medicament for treating a disease and/or disorder in a subject. Finally, it relates to a method for treating a disease or a disorder in a subject comprising administering a therapeutically effective amount of a pharmaceutical composition or a bifunctional molecule according to the invention to the subject.

Disclosed herein, are methods of treating a patient with a disease and/or disorder, the method comprising: (a) identifying a patient in need of treatment; and (b) administering to the patient a therapeutically effective amount of any of the bifunctional molecule, nucleic acid, vector or pharmaceutical composition described herein.

A subject in need of a treatment may be a human having, at risk for, or suspected of having a disease associated with the signaling pathway mediated by PD-1. Such a patient can be identified by routine medical examination. For example, a subject suitable for the treatment can be identified by examining whether such subject carries PD-1, PD-L1 and/or PD-L2 positive cells. Preferably, by “PD-L1 positive tumor cells” or “PD-L2 positive tumor cells” is intended to refer to a population of tumor cells in which PD-L1 or PD-L2, respectively, are expressed in at least 10% of tumor cells, preferable at least 20, 30, 40 or 50% of tumor cells.

In one embodiment, a subject who needs a treatment is a patient having, suspected of having, or at risk for a disease, preferably a PD-1, PDL1 and/or PDL2 positive disease, even more preferably a disease where PD-1 and/or at least one ligand of PD-1 is overexpressed. In such subject, the disruption of PD-1/PD-L1 and/or PD-1/PD-L2 interaction thanks to the administration of the bifunctional molecule or pharmaceutical composition according to the invention may enhance immune response of the subject. In some embodiments, any of the humanized anti-PD-1 antibodies or pharmaceutical composition described herein can be used for treating PD-1 positive cells.

Cancer

It is known in the art that blockade of PD-1 by antibodies can enhance the immune response to cancerous cells in a patient. Thus, in one aspect, the invention provides a bifunctional molecule or a pharmaceutical composition for use in the treatment of a subject having a cancer, comprising administering to the individual an effective amount of the bifunctional molecule or pharmaceutical composition, capable of activating exhausted T cells, and preferably capable of disrupting or inhibiting the PD1/PD-L1 and/or PD1/PD-L2 interaction, and preferably capable of at least partially preventing, disrupting or inhibiting the naturally occurring CD47/SIRPa interaction in a subject.

In one embodiment, a subject who needs a treatment is a patient having, suspected of having, or at risk for a disease, preferably a PD-1 positive cancer, even more preferably a cancer where PD-1 is expressed or overexpressed. In some embodiments, any of the anti-PD-1 antibodies or pharmaceutical composition described herein can be used for treating PD-1 positive tumor cells. For example, a patient suitable for the treatment can be identified by examining whether such a patient carries PD-1 positive tumor cells.

In another embodiment, a subject is a patient having, suspected of having, or at risk for a cancer development, preferably a PD-L1 and/or PD-L2 positive cancer. In some embodiments, any of bifunctional molecule or pharmaceutical composition described herein can be used for treating PD-L1 and/or PD-L2 positive tumors. For example, a human patient suitable for the treatment can be identified by examining whether such a patient carries PD-L1 and/or PD-L2 positive cancer cells.

In another embodiment, a subject is a patient having, suspected of having, or at risk for a cancer development, preferably a CD47 positive cancer. In some embodiments, any of bifunctional molecule or pharmaceutical composition described herein can be used for treating CD47 positive tumors. For example, a human patient suitable for the treatment can be identified by examining whether such a patient carries CD47 positive cancer cells.

In further aspects, a bifunctional molecule or pharmaceutical composition for use in treating cancer, preferably a PD-1, CD47, PD-L1 and/or PD-L2 positive cancer, even more preferably a cancer wherein PD-1, CD47, PD-L1 and/or PD-L2 is/are overexpressed is provided.

In another embodiment, the invention provides the use a bifunctional molecule or pharmaceutical composition as disclosed herein in the manufacture of a medicament for treating a cancer, for instance for inhibiting growth of tumor cells in a subject, preferably PD-1, CD47, PD-L1, PD-L2 positive tumor cells. In an aspect of the disclosure, the cancer to be treated is associated with exhausted T cells.

Accordingly, in one embodiment, the invention provides a method of treating a cancer, for instance for inhibiting growth of tumor cells, in a subject, comprising administering to the subject a therapeutically effective amount of bifunctional molecule or pharmaceutical composition according to the invention. Particularly, the present invention relates to the treatment of a subject using a bifunctional molecule such that growth of cancerous cells is inhibited.

Any suitable cancer may be treated with the bifunctional molecule provided herein can be hematopoietic cancer or solid cancer. Such cancers include carcinoma, cervical cancer, colorectal cancer, esophageal cancer, gastric cancer, gastrointestinal cancer, head and neck cancer, kidney cancer, liver cancer, lung cancer, lymphoma, glioma, mesothelioma, melanoma, stomach cancer, urethral cancer environmentally induced cancers and any combinations of said cancers. The present invention is also useful for treatment of metastatic cancers, especially metastatic cancers that express PD-L1 (Iwai et al. (2005) Int. Immunol. 17: 133-144). Additionally, the invention includes refractory or recurrent malignancies.

In a particular aspect, the cancer is a hematologic malignancy or a solid tumor with high expression of PD-1 and/or PD-L1. Such a cancer can be selected from the group consisting of hematolymphoid neoplasms, angioimmunoblastic T cell lymphoma, myelodysplastic syndrome, acute myeloid leukemia.

In a particular aspect, the cancer is a cancer induced by virus or associated with immunodeficiency. Such a cancer can be selected from the group consisting of Kaposi sarcoma (e.g., associated with Kaposi sarcoma herpes virus); cervical, anal, penile and vulvar squamous cell cancer and oropharyngeal cancers (e.g., associated with human papilloma virus); B cell non-Hodgkin lymphomas (NHL) including diffuse large B-cell lymphoma, Burkitt lymphoma, plasmablastic lymphoma, primary central nervous system lymphoma, HHV-8 primary effusion lymphoma, classic Hodgkin lymphoma, and lymphoproliferative disorders (e.g., associated with Epstein-Barr virus (EBV) and/or Kaposi sarcoma herpes virus); hepatocellular carcinoma (e.g., associated with hepatitis B and/or C viruses); Merkel cell carcinoma (e.g., associated with Merkel cell polyoma virus (MPV)); and cancer associated with human immunodeficiency virus infection (HIV) infection.

Preferably, the cancer to be treated or prevented is selected from the group consisting of metastatic or not metastatic, Melanoma, malignant mesothelioma, Non-Small Cell Lung Cancer, Renal Cell Carcinoma, Hodgkin's Lymphoma, Head and Neck Cancer, Urothelial Carcinoma, Colorectal Cancer, Hepatocellular Carcinoma, Small Cell Lung Cancer Metastatic Merkel Cell Carcinoma, Gastric or Gastroesophageal cancers and Cervical Cancer.

Preferred cancers for treatment include cancers typically responsive to immunotherapy. Alternatively, preferred cancers for treatment are cancers non-responsive to immunotherapy.

By way of example and not wishing to be bound by theory, treatment with an anti-cancer antibody or an anti-cancer immunoconjugate or other current anti-cancer therapy that lead to cancer cell death would potentiate an immune response mediated by PD-1. Accordingly, a treatment of a hyper proliferative disease (e.g., a cancer tumor) may include a bifunctional molecule combined with an anti-cancer treatment, concurrently or sequentially or any combination thereof, which may potentiate an anti-tumor immune response by the host. Preferably, a bifunctional molecule may be used in combination with other immunogenic agents, standard cancer treatments, or other antibodies as described hereafter.

Infectious Disease

The bifunctional molecule, nucleic acid, group of nucleic acid, vector, host cells or pharmaceutical composition of the invention are used to treat patients that have been exposed to particular toxins or pathogens. Accordingly, an aspect of the invention provides a method of treating an infectious disease in a subject comprising administering to the subject a bifunctional molecule according to the present invention, or a pharmaceutical composition comprising such, preferably such that the subject is treated for the infectious disease.

Any suitable infection may be treated bifunctional molecule, nucleic acid, group of nucleic acid, vector, host cells or pharmaceutical composition provided herein.

Some examples of pathogenic viruses causing infections treatable by methods of the invention include HIV, hepatitis (A, B, or C), herpes virus (e.g., VZV, HSV-1, HAV-6, HSV-II, and CMV, Epstein Barr virus), adenovirus, influenza virus, flaviviruses, echovirus, rhinovirus, coxsackie virus, coronavirus, respiratory syncytial virus, mumps virus, rotavirus, measles virus, rubella virus, parvovirus, vaccinia virus, HTLV virus, dengue virus, papillomavirus, molluscum virus, poliovirus, rabies virus, JC virus and arboviral encephalitis virus.

Particularly, the bifunctional molecule or pharmaceutical compositions of the invention are used to treat patients that have chronic viral infection, such infection being caused by viruses selected from the group consisting of Retroviruses, Anellovirus, Circovirus, Herpesvirus, Varicella zoster virus (VZV), Cytomegalovirus (CMV), Epstein-Barr virus (EBV), Polyomavirus BK, Polyomavirus, Adeno-associated virus (AAV), Herpes simplex type 1 (HSV-1), Adenovirus, Herpes simplex type 2 (HSV-2), Kaposi's sarcoma herpesvirus (KSHV), Hepatitis B virus (HBV), GB virus C, Papilloma virus, Hepatitis C virus (HCV), Human immunodeficiency virus (HIV), Hepatitis D virus (HDV), Human T cell leukemia virus type 1 (HTLV1), Xenotropic murine leukemia virus-related virus (XMLV), Rubella virus, German measles, Parvovirus B19, Measles virus, Coxsackie virus.

Some examples of pathogenic bacteria causing infections treatable by methods of the invention include Chlamydia, rickettsial bacteria, mycobacteria, staphylococci, streptococci, pneumonococci, meningococci and conococci, Klebsiella, Proteus, Serratia, Pseudomonas, Legionella, diphtheria, Salmonella, bacilli, cholera, tetanus, botulism, anthrax, plague, leptospirosis, and Lymes disease bacteria.

Some examples of pathogenic fungi causing infections treatable by methods of the invention include Candida (albicans, krusei, glabrata, tropicalis, etc.), Cryptococcus neoformans, Aspergillus (fumigatus, niger, etc.), Genus Mucorales (mucor, absidia, rhizophus), Sporothrix schenkii, Blastomyces dermatitidis, Paracoccidioides brasiliensis, Coccidioides immitis and Histoplasma capsulatum.

Some examples of pathogenic parasites causing infections treatable by methods of the invention include Entamoeba histolytica, Balantidium coli, Naegleriafowleri, Acanthamoeba sp., Giardia lambia, Cryptosporidium sp., Pneumocystis carinii, Plasmodium vivax, Babesia microti, Trypanosoma brucei, Trypanosoma cruzi, Leishmania donovani, Toxoplasma gondi, and Nippostrongylus brasiliensis.

In all of the above methods, the bifunctional molecule can be combined with other forms of immunotherapy such as cytokine treatment (e.g., interferons, GM-CSF, G-CSF, IL-2), or any therapy, which provides for enhanced presentation of tumor antigens.

### Combined Therapy

In particular, bifunctional molecule of the present invention can be combined with some other potential strategies for overcoming immune evasion mechanisms with agents in clinical development or already on the market (see table 1 from Antonia et al. Immuno-oncology combinations: a review of clinical experience and future prospects. Clin. Cancer Res. Off. J. Am. Assoc. Cancer Res. 20, 6258-6268, 2014). Such combination with the bifunctional molecule according to the invention may be useful notably for:
1—Reversing the inhibition of adaptive immunity (blocking T-cell checkpoint pathways);
2—Switching on adaptive immunity (promoting T-cell costimulatory receptor signaling using agonist molecules, in particular antibodies),
3—Improving the function of innate immune cells;
4—Activating the immune system (potentiating immune-cell effector function), for example through vaccine-based strategies.

Accordingly, also provided herein are combined therapies for any of the diseases associated with the PD-1 signaling and/or SIRPa signaling as described herein with any of the bifunctional molecule or pharmaceutical composition comprising such, as described herein and a suitable second agent. In an aspect, the bifunctional molecule and the second agent can be present in a pharmaceutical composition as described above. Alternatively, the terms “combination therapy” or “combined therapy”, as used herein, embrace administration of these two agents (e.g., a bifunctional molecule as described herein and an additional or second suitable therapeutic agent) in a sequential manner, that is, wherein each therapeutic agent is administered at a different time, as well as administration of these therapeutic agents, or at least two of the agents, in a substantially simultaneous manner. Sequential or substantially simultaneous administration of each agent can be affected by any appropriate route. The agents can be administered by the same route or by different routes. For example, a first agent (e.g., a bifunctional molecule) can be administered orally, and an additional therapeutic agent (e.g., an anti-cancer agent, an anti-infection agent; or an immune modulator) can be administered intravenously. Alternatively, an agent of the combination selected may be administered by intravenous injection while the other agents of the combination may be administered orally.

In another aspect, the invention relates to a therapeutic mean, in particular a combination product mean, which comprises as active ingredients: a bifunctional molecule as defined above and an additional therapeutic agent, wherein said active ingredients are formulated for separate, sequential or combined therapy, in particular for combined or sequential use.

As used herein, the term “sequential” means, unless otherwise specified, characterized by a regular sequence or order, e.g., if a dosage regimen includes the administration of a bifunctional molecule and the second agent, a sequential dosage regimen could include administration of the bifunctional molecule of the invention before, simultaneously, substantially simultaneously, or after administration of the second agent, but both agents will be administered in a regular sequence or order. The term “separate” means, unless otherwise specified, to keep apart one from the other. The term “simultaneously” means, unless otherwise specified, happening or done at the same time, i.e., the agents of the invention are administered at the same time. The term “substantially simultaneously” means that the agents are administered within minutes of each other (e.g., within 15 minutes of each other) and intends to embrace joint administration as well as consecutive administration, but if the administration is consecutive it is separated in time for only a short period (e.g., the time it would take a medical practitioner to administer two compounds separately).

It should be appreciated that any combination as described herein may be used in any sequence for treating the disorder or disease described herein. The combinations described herein may be selected on the basis of a number of factors, which include but are not limited to the effectiveness of inhibiting or preventing the target disease progression, the effectiveness for mitigating the side effects of another agent of the combination, or the effectiveness of mitigating symptoms related to the target disease. For example, a combined therapy described herein may reduce any of the side effects associated with each individual members of the combination.

The present invention also relates to a method for treating a disease in a subject comprising administering to said subject a therapeutically effective amount of the bifunctional molecule or the pharmaceutical composition described herein and a therapeutically effective amount of an additional or second therapeutic agent.

When the bifunctional molecule or the pharmaceutical composition described here is co-used with an additional therapeutic agent, a sub-therapeutic dosage of either the bifunctional molecule, the pharmaceutical composition or of the second agent, or a sub-therapeutic dosage of both, can be used in the treatment of a subject, preferably a subject having, or at risk of developing a disease or disorder associated with the cell signaling mediated by PD-1 and/or SIRPa.

Specific examples of additional or second therapeutic agents are provided in WO 2018/053106, pages 36-43.

In an aspect, the additional or second therapeutic agent can be selected in the non-exhaustive list comprising alkylating agents, angiogenesis inhibitors, antibodies, antimetabolites, antimitotics, antiproliferatives, antivirals, aurora kinase inhibitors, apoptosis promoters (for example, Bcl-2 family inhibitors), activators of death receptor pathway, Bcr-Abl kinase inhibitors, BiTE (Bi-Specific T cell Engager) antibodies, antibody drug conjugates, biologic response modifiers, Bruton's tyrosine kinase (BTK) inhibitors, cyclin-dependent kinase inhibitors, cell cycle inhibitors, cyclooxygenase-2 inhibitors, DVDs, leukemia viral oncogene homolog (ErbB2) receptor inhibitors, growth factor inhibitors, heat shock protein (HSP)-90 inhibitors, histone deacetylase (HDAC) inhibitors, hormonal therapies, immunologicals, inhibitors of inhibitors of apoptosis proteins (IAPs), intercalating antibiotics, kinase inhibitors, kinesin inhibitors, Jak2 inhibitors, mammalian target of rapamycin inhibitors, microRNAs, mitogen-activated extracellular signal-regulated kinase inhibitors, multivalent binding proteins, non-steroidal anti-inflammatory drugs (NSAIDs), poly ADP (adenosine diphosphate)-ribose polymerase (PARP) inhibitors, platinum chemotherapeutics, polo-like kinase (Plk) inhibitors, phosphoinositide-3 kinase (PI3K) inhibitors, proteasome inhibitors, purine analogs, pyrimidine analogs, receptor tyrosine kinase inhibitors, retinoids/deltoids plant alkaloids, small inhibitory ribonucleic acids (siRNAs), topoisomerase inhibitors, ubiquitin ligase inhibitors, hypomethylating agents, checkpoints inhibitors, peptide vaccine and the like, epitopes or neoepitopes from tumor antigens, as well as combinations of one or more of these agents.

For instance, the additional therapeutic agent can be selected in the group consisting of chemotherapy, radiotherapy, targeted therapy, antiangiogenic agents, hypomethylating agents, cancer vaccines, epitopes or neoepitopes from tumor antigens, myeloid checkpoints inhibitors, other immunotherapies, and HDAC inhibitors.

In a preferred embodiment, the second therapeutic agent is selected from the group consisting of chemotherapeutic agents, radiotherapy agents, immunotherapeutic agents, cell therapy agents (such as CAR-T cells), antibiotics and probiotics. Said immunotherapeutic agent can also be an antibody targeting tumoral antigen, particularly selected from the group consisting of anti-Her2, anti-EGFR, anti-CD20, anti-CD19, anti-CD52.

In an embodiment, the invention relates to a combined therapy as defined above, wherein the second therapeutic agent is particularly selected from the group consisting of therapeutic vaccines, immune checkpoint blockers or activators, in particular of adaptive immune cells (T and B lymphocytes) and antibody-drug conjugates. Preferably, suitable agents for co-use with any of the anti-hPD-1 antibodies or fragment thereof or with the pharmaceutical composition according to the invention include an antibody binding to a co-stimulatory receptor (e.g., OX40, CD40, ICOS, CD27, HVEM or GITR), an agent that induces immunogenic cell death (e.g., a chemotherapeutic agent, a radio-therapeutic agent, an anti-angiogenic agent, or an agent for targeted therapies), an agent that inhibits a checkpoint molecule (e.g., CTLA4, LAG3, TIM3, B7H3, B7H4, BTLA, or TIGIT), a cancer vaccine, an agent that modifies an immunosuppressive enzyme (e.g., IDO1 or iNOS), an agent that targets T_{reg} cells, an agent for adoptive cell therapy, or an agent that modulates myeloid cells.

In an embodiment, the invention relates to a combined therapy as defined above, wherein the second therapeutic agent is an immune checkpoint blocker or activator of adaptive immune cells (T and B lymphocytes) selected from the group consisting of anti-CTLA4, anti-CD2, anti-CD28, anti-CD40, anti-HVEM, anti-BTLA, anti-CD160, anti-TIGIT, anti-TIM-1/3, anti-LAG-3, anti-2B4, and anti-OX40, anti-CD40 agonist, CD40-L, TLR agonists, anti-ICOS, ICOS-L and B-cell receptor agonists.

In one embodiment, the second therapeutic agent is an antibody targeting tumoral antigen, particularly selected from the group consisting of anti-Her2, anti-EGFR, anti-CD20, anti-CD19, anti-CD52. Combination therapy could also rely on the combination of the administration of bifunctional molecule with surgery, chemotherapy (e.g. such as docetaxel or decarbazine), radiotherapy, immunotherapy (e.g. such as antibodies targeting CD40, CTLA-4), gene targeting and modulation, and/or other agents such as immune-modulators, angiogenesis inhibitor and any combinations thereof.

### Kits

Any of the bifunctional molecules or compositions described herein may be included in a kit provided by the present invention. The present disclosure particularly provides kits for use in enhancing immune responses and/or treating diseases (e.g. cancer and/or infection) associated with the PD-1 signaling, SIRPa/CD47 signaling.

In the context of the present invention, the term “kit” means two or more components (one of which corresponding to the bifunctional molecule, the nucleic acid molecule, the vector or the cell of the invention) packaged in a container, recipient or otherwise. A kit can hence be described as a set of products and/or utensils that are sufficient to achieve a certain goal, which can be marketed as a single unit.

Particularly, a kit according to the invention may comprise:
a bifunctional molecule as defined above,
an anti-hPD1 antibody or antigen-binding fragment thereof linked to SIRPa or a variant thereof,
a nucleic acid molecule or a group of nucleic acid molecules encoding said bifunctional molecule,
a vector comprising said nucleic acid molecule or group of nucleic acid molecules, and/or
a cell comprising said vector or nucleic acid molecule or group of nucleic acid molecules.

The kit may thus include, in suitable container means, the pharmaceutical composition, and/or the bifunctional molecules, and/or host cells of the present invention, and/or vectors encoding the nucleic acid molecules of the present invention, and/or nucleic acid molecules or related reagents of the present invention. In some embodiments, means of taking a sample from an individual and/or of assaying the sample may be provided. In certain embodiments the kit includes cells, buffers, cell media, vectors, primers, restriction enzymes, salts, and so forth. The kits may also comprise means for containing a sterile, pharmaceutically acceptable buffer and/or other diluent.

The containers may be unit doses, bulk packages (e.g., multi-dose packages) or sub-unit doses. In an embodiment, the invention relates to a kit as defined above for a single-dose administration unit. The kit of the invention may also contain a first recipient comprising a dried/lyophilized bifunctional molecule and a second recipient comprising an aqueous formulation. In certain embodiments of this invention, kits containing single-chambered and multi-chambered pre-filled syringes (e.g., liquid syringes and lyosyringes) are provided.

The kits of this invention are in suitable packaging. Suitable packaging includes, but is not limited to, vials, bottles, jars, flexible packaging (e.g., sealed Mylar or plastic bags), and the like. Also contemplated are packages for use in combination with a specific device, such as an inhaler, nasal administration device (e.g., an atomizer) or an infusion device such as a minipump. A kit may have a sterile access port (for example the container may be an intravenous solution bag or a vial having a stopper penetrable by a hypodermic injection needle). The container may also have a sterile access port (for example the container may be an intravenous solution bag or a vial having a stopper penetrable by a hypodermic injection needle). At least one active agent in the composition is a bifunctional molecule as described herein comprising an anti-hPD1 antibody linked to SIRPa or a variant thereof.

The compositions comprised in the kit according to the invention may also be formulated into a syringe compatible composition. In this case, the container means may itself be a syringe, pipette, and/or other such like apparatus, from which the formulation may be applied to an infected area of the body, and/or even applied to and/or mixed with the other components of the kit. The components of the kit may alternatively be provided as dried powder(s). When reagents and/or components are provided as a dry powder, a soluble composition can be reconstituted by the addition of a suitable solvent. It is envisioned that the solvent may also be provided in another container means and be suitable for administration.

In some embodiments, the kit further includes an additional agent for treating cancer or an infectious disease, and the additional agent may be combined with the bifunctional molecule, or other components of the kit of the present invention or may be provided separately in the kit. Particularly, the kit described herein may include one or more additional therapeutic agents such as those described in the “Combined Therapy” described hereabove. The kit(s) may be tailored to a particular cancer for an individual and comprise respective second cancer therapies for the individual as described hereabove.

The instructions related to the use of the bifunctional molecule or pharmaceutical composition described herein generally include information as to dosage, dosing schedule, route of administration for the intended treatment, means for reconstituting the bifunctional molecule and/or means for diluting the bifunctional molecule of the invention. Instructions supplied in the kits of the invention are typically written instructions on a label or package insert (e.g., a paper sheet included in the kit in the form of a leaflet or instruction manual). In some embodiments, the kit can comprise instructions for use in accordance with any of the methods described herein. The included instructions can comprise a description of administration of the pharmaceutical composition comprising the bifunctional molecule to enhance immune responses and/or to treat a disease as described herein. The kit may further comprise a description of selecting an individual suitable for a treatment based on identifying whether that individual has a disease associated with the PD-1 signaling, e.g., those described herein.

### EXAMPLES

The following Figures and Examples are put forth so as to provide those of ordinary skill in the art with a complete disclosure and description of how to make and use the present invention, and are not intended to limit the scope of what the inventors regard as their invention nor are they intended to represent that the experiments below are all or the only experiments performed. While the present invention has been described with reference to the specific embodiments thereof, it should be understood by those skilled in the art that various changes may be made and equivalents may be substituted without departing from the true spirit and scope of the invention. In addition, many modifications may be made to adapt a particular situation, material, composition of matter, process, process step or steps, to the objective, spirit and scope of the present invention. All such modifications are intended to be within the scope of the claims appended hereto.

### Bifunctional Molecules (Bicki)

Bifunctional molecule comprising an anti-PD-1 antibody and human SIRPa, the protein being covalently linked to a polypeptide chain of the anti-PD-1 antibody, either the light chain (anti-PD1VL-SIRPa antibody) or the heavy chain (anti-PD1VH-SIRPa antibody) of the antibody.

### Example 1: Effect of the Bifunctional Molecules Anti-PD1-SIRPa on the Binding to PD1 and its Antagonist Capacity on PD1-PDL1 Interaction

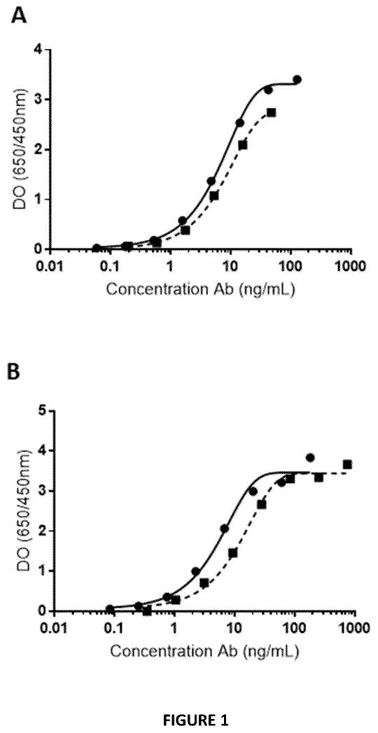

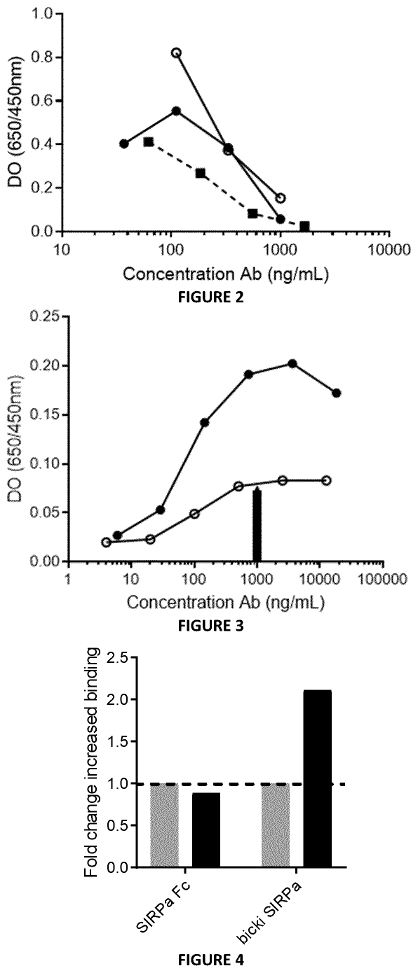

The binding capacity of the Bicki molecules to PD1 recombinant molecule was assessed and the inhibitory efficacy of the Bicki molecules on PD1-PDL1 interaction was performed by ELISA. Results are presented in FIGS. 1 and 2. Bicki anti-PD1-Sirpa molecules where SIRPa is fused to the heavy or the light chain of the antibody does not modify the binding to PD1. Bicki anti-PD1-Sirpa molecules are still capable to inhibit PD1-PDL1 interaction compared to an anti-PD1 antibody alone. No significant difference was observed between Bicki molecules fused to heavy or light chain of the antibody.

These data were confirmed by surface plasmon resonance experiment (Biacore assay), an anti-human Fc antibody on the sensor chip to capture anti PD-1 alone or the bifunctional molecule were. Then, different concentrations of PD-1 recombinant protein (6.25 to 100 nM) were added to measure the affinity. The anti PD-1 alone demonstrates similar high affinity to PD-1 with a KD of 3.46 nM compared to the anti-PD1-Sirpa bifunctional molecule (3.83 nM).

### Example 2: Binding to CD47 of the Bicki Anti-PD1-Sirpa Molecules

The binding capacity of Bicki anti-PD1-Sirpa molecules to CD47 (SIRPa Ligand) was assessed by ELISA. Results presented in FIG. 3 show that Bicki anti-PD1-Sirpa molecules conserved their capacity to bind the SIRPa ligand, i.e., CD47. Surprisingly, a higher efficacy has been observed for the Bicki anti-PD1VH-Sirpa molecules compared to the Bicki anti-PD1VL-Sirpa molecule.

### Example 3: Binding of the Molecule BiCKI SIRPa on T Cells Expressing Both Receptor CD47 and PD1

The capacity of the BiCKI SIRPa to target T cell by binding both CD47 and PD-1 proteins on the same cells was assessed. Jurkat cells expressing CD47 receptor only or co-expressing PD-1 and CD47 proteins were incubated with the BiCKI SIRPa molecule or SIRPa-Fc molecule. The binding was revealed with an anti-human IgG Fc-PE. FIG. 4 confirms the mechanism of the BiCKI SIRPa acting on the same T cell because the molecule binds with 2-fold higher efficacy to cells expressing CD47+PD-1+ compared to cells expressing only CD47. These experiments demonstrate that the bifunctional Bicki SIRPa molecule is designed to preferentially target CD47+PD-1+ exhausted T cells over other CD47+ cells.

### Example 4: In Vitro and Ex Vivo Efficacy of Bicki Anti-PD1-Sirpa Molecules on PBMCs and T Cells Proliferation and Activation

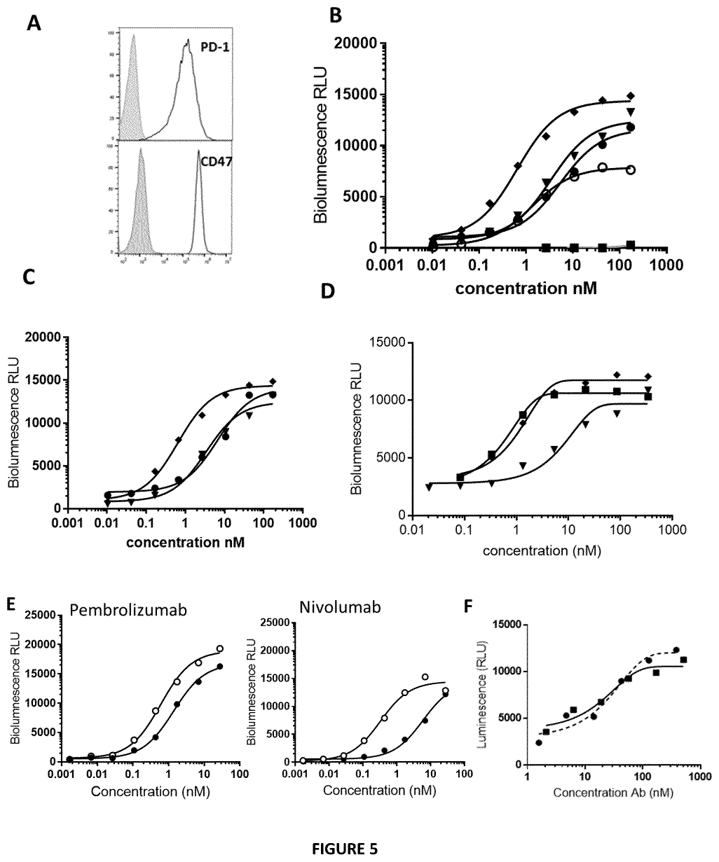

In vitro bioassay to measure T cell activation was performed to compare Bicki anti-PD1-Sirpa molecule with anti PD-1 alone. First of all, expression of PD1 and CD47 was measured by FACS on T cell lines used in the bioassay. FIG. 5A shows a good expression of both PD1 and CD47 molecules at the cell surface. Unexpectedly, in FIG. 5B, inventors observed that the Bicki anti-PD1-Sirpa molecule (EC50=0.6 nM) induces a better NFATTCR-mediated activation than the anti-PD1 antibody alone (EC50=5 nM) (FIG. 5B). In a surprising manner, the inventors also observed that the Bicki SIRPa is more efficient in activating NFAT signaling compared to the combination anti PD-1+isotype SIRPa, demonstrating that the fusion of the anti PD-1 to SIRPa in the Bicki molecule achieves a synergistic effect to activate T cells. This effect requires the activation of CD47 mediated signaling as the use of CD47 blocking antibody (clone B6H12) completely abolished the synergistic effect of the BiCKI SIRPa (FIG. 5B). By targeting the PD-1 receptor, the anti PD-1 domain of the Bicki molecule allows the crosslinking of SIRPa and subsequently the clusterization of the CD47 molecule on T cells. The CD47 mediated signaling strengthens the anti PD-1 effect leading to a better T cell activation. As shown on FIG. 5D, similar synergistic effect was observed with Bicki SIRPa molecule constructed with an IgG1 N298A or IgG4 S228P isotype.

The inventors have constructed the Bicki SIRPa molecule with other anti PD-1 backbone (Pembrolizumab or Nivolumab). FIG. 5E demonstrates that these Bicki molecules possess similar synergistic effect compared to the anti PD-1 alone, suggesting that the invention can be suitable for other anti PD-1 backbones. The bioassay was also performed with a Bicki anti-PD1 fused on the heavy chain to another type 1 protein. Part F of FIG. 5 indicates almost no difference between control anti-PD1 and Bicki anti-PD1VH-Type I protein on NFAT activation, indicating that the enhanced effect observed is specific for the Bicki SIRPa molecule and is not applicable for any Bicki molecule.

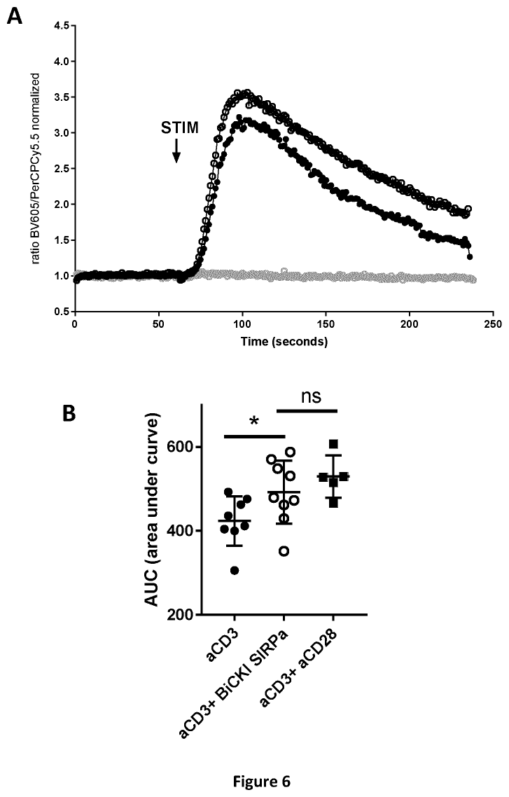

In another bioassay, the inventors assessed the efficacy of the Bicki SIRPa molecule to stimulate calcium signal in T cell, another essential mediator for activation of T cell effector functions. FIGS. 6 A and B show that the Bicki SIRPa molecules potentiate calcium signal induced by aCD3 stimulation to similar extend to the CD28 stimulation. Interestingly, the Bicki SIRPa molecule alone has no effect (FIG. 6A) suggesting that the Bicki SIRPa molecule acts as costimulatory signal and will only promote the activation of TCR engaged T cells.

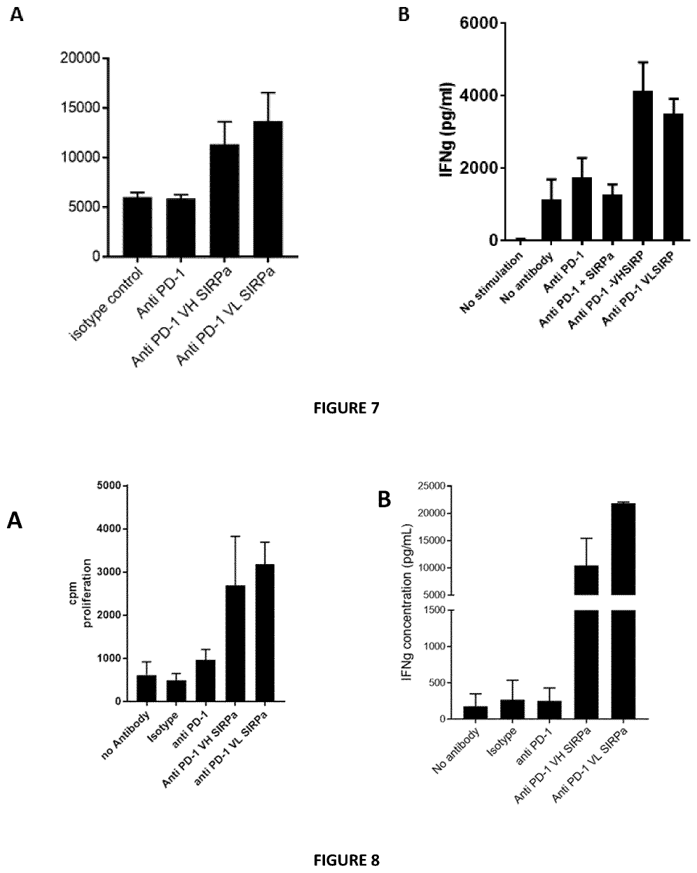

Altogether, these results indicate that the bifunctional molecules with Sirpa fused to the anti-PD1 antibody potentiate anti-PD1 effect and strongly suggest that SIRPa binding on CD47 not only blocks “don't EAT-ME” inhibitory phagocytic signals but also promotes CD47-dependent T-cell costimulation. Ex vivo assays were performed on human PBMCs and T cells to study the efficacy of Bicki anti-PD1-Sirpa molecule on proliferation and activation. Results are presented FIGS. 7 and 8. These results indicate clearly and surprisingly that the Bicki anti-PD1-Sirpa molecule increased human PBMCs proliferation and activation as reflected by IFNg secretion, while it is not the case with anti-PD1 alone or using the combination of the anti-PD1 with a separate recombinant human SIRPa protein, confirming the synergy of fusing SIRPa on the anti-PD1 into a Bicki molecule to increase T-cell proliferation and activation. Results on human T cells confirmed that Bicki anti-PD1-Sirpa molecules enhance T cell proliferation and activate T cells better than anti-PD1 alone. In particular, the amount of IFNg secretion (a key determinant observed to predict the anti-PD1 efficacy in various clinical trials) induced by the Bicki anti-PD1-Sirpa molecules is very high (>10 000 pg/ml) as compared to anti-PD1 alone (<500 pg/ml).

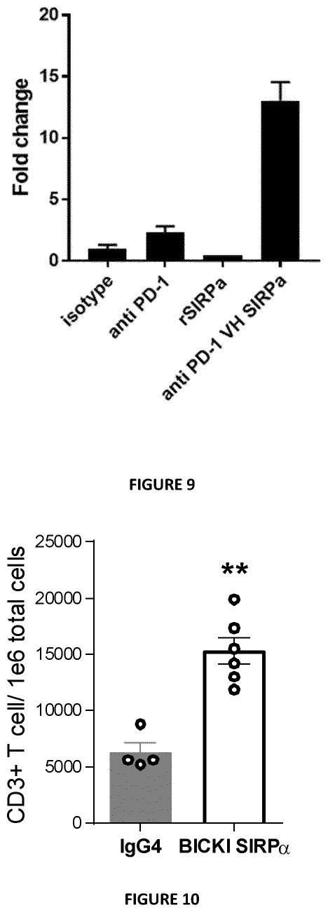

FIG. 9 confirms the potent synergistic effect to stimulate the proliferation of exhausted human T cells after chronic antigen stimulation. Indeed, SIRPa recombinant protein or the anti PD-1 alone does not induces T-cell proliferation as compared to isotype control whereas, surprisingly, the Bicki anti-PD1-Sirpa molecule strongly stimulates the proliferation of exhausted T cells. This difference highlights the advantage of the bifunctional antibody versus combination therapy using two separate molecules. Bicki anti-PD1-Sirpa molecules of the invention dock the molecule on PD1+ cell clustering SIRPa molecules thereby stimulating CD47 signaling into T cells and proliferation of T cells.

### Example 5: Bicki Anti-PD1-Sirpa Molecules Potentiate T Cell Migration into the Tumor

Migration of the T cells was investigated using 3D tumor spheroid-based assay. Tumor spheroids were generated by coculturing A549 tumor cells with fibroblast and monocytes to mimic the complexity of the solid tumor microenvironment. Human T cells were added to the well and T cell migration into the tumor spheroids was assessed by immunofluorescence and confocal microscopy analysis. FIG. 10 shows that the treatment with BiCKI SIRPa molecules enhances number of T cells/tumor cell into the tumor compared to the isotype control treatment. The lack of T cell into the tumor microenvironment is one of the major resistance mechanisms associated to the anti PD-1 monotherapy. These data suggest that the BiCKi SIRPa molecules can overcome this resistance by enhancing the migration of the T cells into the tumor microenvironment.

### Example 6: Pharmacokinetics Properties of the Bicki Anti-PD1 SIRPa Molecule In Vivo

To analyze the pharmacokinetics of the Bicki molecules, BalbcRJ (female 6-9 weeks) were intra-orbitally treated with a single dose of the BiCKi SIRPa molecules. Bicki molecules concentration in the plasma was assessed by ELISA using an immobilized anti-human light chain antibody (clone NaM76-5F3), and the diluted serum containing anti-PD-1 antibody was added. Detection was performed with a peroxidase-labeled donkey anti-human IgG (Jackson Immunoresearch; USA; reference 709-035-149) and revealed by conventional methods.

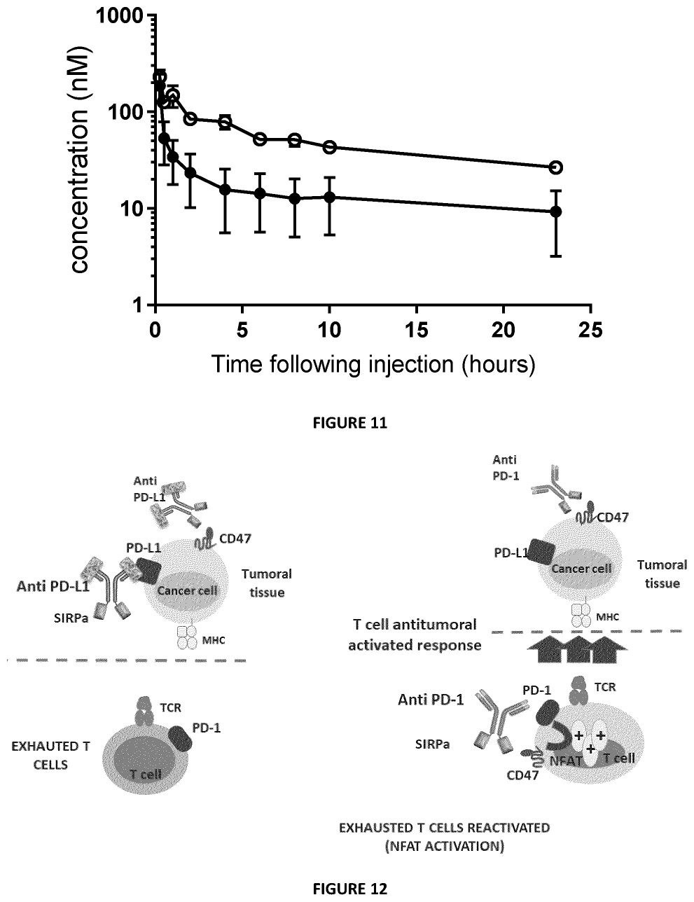

In this assay, two different Bicki SIRPa molecules were tested and compared (1) BiCKI SIRPa molecule with an IgG1 N298A isotype and a GGGGS linker between the Fc and SIRPa domain (2) BiCKI SIRPa molecule with an IgG4 S228P and a GGGGSGGGGSGGGGS linker. A linear pharmacokinetic profile for both molecules is observed with a similar absorption phase (FIG. 11). A Cmax around 200 nM was obtained for both constructions at 15 minutes following injection. However, surprisingly, the biCKI SIRPa molecule with GGGS and IgG1 isotype depicted a lower elimination/distribution phase compared to the BiCKI SIRPa molecule constructed with an IgG4 backbone and a long linker. These data suggest the use of an IgG1 N298A along with a short linker for Bicki SIRPa construction may prolong drug exposure in vivo and subsequently enhance therapeutics efficacy of the drug.

### Material and Methods

### ELISA Binding PD1 and Bridging ELISA Assay

For the PD-1 binding ELISA assay, recombinant hPD1 (Sino Biologicals, Beijing, China; reference 10377-H08H) was immobilized on plastic at 0.5 μg/ml in carbonate buffer (pH 9.2) and purified antibody were added to measure binding. After incubation and washing, peroxidase-labeled donkey anti-human IgG (Jackson Immunoresearch; USA; reference 709-035-149) was added and revealed by conventional methods.

For bridging ELISA assay, a similar method was used. Recombinant hPD1 was immobilized and purified bifunctional antibodies were added at serial dilution. After incubation and washing, CD47fc recombinant protein (Sino Biologicals, reference 12283-H02H) were then added at 1 μg/mL. Detection was performed using an anti-CD47 specific mouse antibody (clone B6H12) and a peroxidase-labeled donkey anti mouse IgG antibody (Reference 715-036-151). Revelation was performed using conventional method.

### ELISA Antagonist: Competition Between PDL1 and Humanized Anti-PD1

Competitive ELISA assay was performed by PD-1:PD-L1 Inhibitor Screening ELISA Assay Pair (AcroBiosystems; USA; reference EP-101). In this assay, recombinant hPDL1 was immobilized on plastic at 2 μg/ml in PBS pH 7.4 buffer. Purified antibody (at different concentrations) were mixed with 0.66 μg/ml final (fix concentration) of biotinylated Human PD1 (AcroBiosystems; USA; reference EP-101) to measure competitive binding for 2 h at 37° C. After incubation and washing, peroxidase-labeled streptavidin (Vector laboratoring; USA; reference SA-5004) was added to detect Biotin-CD47Fc binding and revealed by conventional methods.

### IFN Gamma Secretion and T Cell Proliferation Assay

Peripheral blood mononuclear cells or purified T cells isolated from healthy donors were used for the experiments.

For experiment using naïve PBMCs, PBMCs were incubated on anti CD3+/−CD28 (clone OKT3 and CD28.2, 3 ug/mL) coated plate with an isotype control (B12G4M), anti PD-1, anti PD-1+rSIRPa (Sino Biologicals, Reference 11612-H08H), BiCKI anti PD-1 VH SIRPa or BiCKI anti PD-1 VH SIRPa. IFNg was dosed by ELISA in the supernatant harvested on Day 2 (human IFNg ELISA set, BD Bioscience, USA, reference 555142). Proliferation was assessed by H3 thymidine incorporation on Day 6. T cells were stimulated with anti-CD3 (clone OKT3, 3 ug/mL with or without anti-CD28 (clone CD28.2, 3 ug/mL).

For experiment using activated T cells, T cells were firstly stimulated on anti-CD3/CD28 coated plate (3 ug/mL each). Twenty-four hours following stimulation, T cells were harvested, counted and restimulated on anti-CD3 (clone OKT3, 2 ug/mL)+recombinant human PD-L1 (Sinobiological, reference 10084-H02H, 5 ug/mL) in the presence of an isotype control, anti-PD-1 or BiCKI VH SIRPa and BiCKI VL SIRPa (10 ug/mL). At Day 6, proliferation was quantified by H3 thymidine incorporation and supernatant was harvested to quantify IFNg secretion.

For experiment using chronically stimulated PBMCs, Human PBMCs were repeatedly stimulated on CD3 CD28 coated plate (3 ug/mL of OKT3 and 3 ug/mL CD28.2 antibody) every 3 days. After the 3rd stimulations, T cells were incubated with an isotype control or an anti PD-1, a recombinant SIRPa protein or BiCKI anti PD-1 VH SIRPa (5 ug/mL). H3 incorporation assay was performed on Day 5 to determine T cell proliferation.

### T Cell Activation Assay Using Promega Cell-Based Bioassay

The capacity of anti-PD-1 antibodies restore T cell activation was tested using Promega PD-1/PD-L1 kit (Reference J1250). Two cell lines are used (1) Effector T cells (Jurkat stably expressing PD-1, NFAT-induced luciferase) and (2) activating target cells (CHO K1 cells stably expressing PDL1 and surface protein designed to stimulate cognate TCRs in an antigen-independent manner. When cells are cocultured, PD-L1/PD-1 interaction inhibits TCR mediated activation thereby blocking NFAT activation and luciferase activity. The addition of an anti-PD-1 antibody blocks the PD-1 mediated inhibitory signal leading to NFAT activation and luciferase synthesis and emission of bioluminescence signal. Experiment was performed as per as manufacturer recommendations. Serial dilutions of the PD-1 antibody were tested. Four hours following coculture of PD-L1+ target cells, PD-1 effector cells and anti PD-1 antibodies, BioGlo™ luciferin substrate was added to the wells and plates were read using Tecan™ luminometer.

### Calcium Flux Assay

CD47+PD1+ Jurkat cells were stained with Fura-red (Thermofisher, #F3021, 5 uM) 30 min at 37° C. in HBSS media, then washed twice with HBSS media supplemented with HEPES (10 mM), BSA 1% and CaCl2) (1 mM) and resuspended with the same media. After acquisition of 1 minute on LSR FACS to set up the fluorescence background, Bicki anti-PD1-SIRPa antibodies alone (45 nM 10 ug/mL) or mixed with CD3 antibody (clone OKT3, 10×10 ug/mL) was added on the cells. The ratio BV711/PercP5.5 MFI was calculated per second of acquisition and normalized to 1 corresponding to the MFI before stimulation (mean of 20 seconds). AUC (Area under the curve) was calculated and reported for each stimulation using Graph pad Software.

Pharmacokinetics of the biCKI Sirpa In Vivo

To analyze the pharmacokinetics, BalbcRJ (female 6-9 weeks) were intra-orbitally with a single dose of the molecule. Drug concentration in the plasma was determined by ELISA using an immobilized anti-human light chain antibody (clone NaM76-5F3). Detection was performed with a peroxidase-labeled donkey anti-human IgG (Jackson Immunoresearch; USA; reference 709-035-149) and revealed by conventional methods.

3D-Spheroid Migration Assay 3D cell cultures were established in 96-wells U-bottom low attachment plates (6055330 Perkin Elmer) and seeded 5000, 1500 and 10 000 cells/wells for A549, MRC-5 and fresh monocytes, respectively. Spheroids were formed and incubated with GM-CSF (10 ng/ml) in complete RPMI. Cells were treated with Isotype (IgG4) or BICKI-Sirpa (50 nM) for 3 days. At day 3, 250 000 T cells/wells were added on spheroids. After 72 h co-culture, spheroids were fixed 15 min in PFA 4% at room temperature, washed 3 times in PBS and kept at 4° C. in PBS-FBS-EDTA buffer until staining. Then, spheroids were permeabilized in PBS-0,5% Triton and after 1 hour of saturation step in PBS-0,1% Triton-1% MA at RT, spheroids were incubated with primary rabbit anti-human CD3 (A045229-2; 6 μg/ml, staining ON 4° C.) then 2 hours at RT with secondary Donkey ant-rabbit A488 antibody (10 μg/ml) and DAPI (10 μg/ml). A A1RSi confocal microscope (Nikon) was used for fluorescence detection and Confocal z-slice images were analyzed via FIJI software using morpholibJ, LoG and 3D-suite plug-in.

### Antibodies and Bifunctional Molecules

The following antibodies and bifunctional molecules have been used in the different experiments disclosed herein: Pembrolizumab (Keytrudra, Merck) Nivolumab (Opdivo, Bristol-Myers Squibb), and the bifunctional molecules as disclosed herein comprising an anti-PD1 humanized antibody comprising a heavy chain variable domain as defined in SEQ ID:19, 22 or 24 and a light chain variable domain as defined in SEQ ID NO: 28 or an anti-PD1 chimeric antibody comprising a heavy chain as defined in SEQ ID NO: 53 and a light chain as defined in SEQ ID NO: 54.

1-22. (canceled)

23. A bifunctional molecule comprising:
(a) an anti-human PD-1 antibody or an antigen-binding fragment thereof, which comprises:
(i) a heavy chain variable domain (VH) comprising a HCDR1, a HCDR2 and a HCDR3, and
(ii) a light chain variable domain (VL) comprising a LCDR1, a LCDR2 and a LCDR3, and
(b) a human SIRPa or a fragment or variant thereof,
wherein the C-terminal end of the heavy and/or light chain(s) of the antibody or antigen-binding fragment thereof is covalently linked to the N-terminal end of the SIRPa or fragment or variant thereof as a fusion protein.

24. The bifunctional molecule of claim 23, wherein the antibody is a chimeric, a humanized or a human antibody.

25. The bifunctional molecule of claim 23, wherein the SIRPa fragment comprises or consists of the extracellular domain of SIRPa.

26. The bifunctional molecule of claim 23, wherein the SIRPa fragment is devoid of the intracellular part thereof and optionally of the transmembrane domain thereof, or wherein the SIRPa comprises or consists of the amino acid sequence set forth in SEQ ID NO: 51 or a fragment thereof.

27. The bifunctional molecule of claim 23, wherein the anti-human PD-1 antibody or antigen-binding fragment thereof, comprises:
(i) a heavy chain variable domain (VH) comprising HCDR1, HCDR2 and HCDR3, and
(ii) a light chain variable domain (VL) comprising LCDR1, LCDR2 and LCDR3,
wherein:
the heavy chain CDR1 (HCDR1) comprises or consists of an amino acid sequence of SEQ ID NO: 1;
the heavy chain CDR2 (HCDR2) comprises or consists of an amino acid sequence of SEQ ID NO: 2;
the heavy chain CDR3 (HCDR3) comprises or consists of an amino acid sequence of SEQ ID NO: 3 wherein X1 is D or E and X2 is selected from the group consisting of T, H, A, Y, N, E and S;
the light chain CDR1 (LCDR1) comprises or consists of an amino acid sequence of SEQ ID NO: 12 wherein X is G or T;
the light chain CDR2 (LCDR2) comprises or consists of an amino acid sequence of SEQ ID NO: 15;
the light chain CDR3 (LCDR3) comprises or consists of an amino acid sequence of SEQ ID NO:16.

28. The bifunctional molecule of claim 23, wherein the anti-human PD-1 antibody or antigen-binding fragment thereof, comprises or consists of: (a) a VH comprising or consisting of an amino acid sequence of SEQ ID NO: 17, wherein X1 is D or E and X2 is selected from the group consisting of T, H, A, Y, N, E and S; and (b) a VL comprising or consisting of an amino acid sequence of SEQ ID NO: 26, wherein X is G or T.

29. The bifunctional molecule of claim 23, wherein the antibody or antigen-binding fragment thereof comprises a light chain constant domain derived from a human kappa light chain constant domain and a heavy chain constant domain derived from a human IgG1, IgG2, IgG3 or IgG4 heavy chain constant domain.

30. The bifunctional molecule of claim 23, wherein the antibody or antigen-binding fragment thereof comprises a light chain constant domain derived from a human kappa light chain constant domain and a heavy chain constant domain derived from a human IgG1 heavy chain constant domain, optionally with a substitution or a combination of substitutions selected from the group consisting of T250Q/M428L; M252Y/S254T/T256E+H433K/N434F; E233P/L234V/L235A/G236A+A327G/A330S/P331S; E333A; S239D/A330L/I332E; P257I/Q311; K326W/E333S; S239D/I332E/G236A; N297A; L234A/L235A; N297A+M252Y/S254T/T256E; K322A; and K444A.

31. The bifunctional molecule of claim 23, wherein the antibody or antigen-binding fragment thereof comprises a light chain constant domain derived from a human kappa light chain constant domain and a heavy chain constant domain derived from a human IgG4 heavy chain constant domain, optionally with a substitution or a combination of substitutions selected from the group consisting of S228P; L234A/L235A, S228P+M252Y/S254T/T256E and K444A.

32. The bifunctional molecule of claim 23, wherein, the anti-PD1 antibody is be selected from the group consisting of Pembrolizumab, Nivolumab, Pidilizumab, Cemiplimab, PDR001, and monoclonal antibodies 5C4, 17D8, 2D3, 4H1, 4A11, 7D3, and 5F4.

33. An isolated nucleic acid molecule or a group of isolated nucleic acid molecules encoding the bifunctional molecule according to claim 23.

34. A vector comprising the nucleic acid or group of nucleic acid molecules according to claim 33.

35. A host cell comprising the nucleic acid or group of nucleic acid molecules of claim 33 or a vector comprising said nucleic acid or group of nucleic acids.

36. A method for producing the bifunctional molecule comprising a step of culturing a host cell according to claim 35 and optionally a step of isolating the bifunctional molecule.

37. A pharmaceutical composition comprising the bifunctional molecule according to claim 23 and a pharmaceutically acceptable carrier.

38. The pharmaceutical composition of claim 37, wherein the pharmaceutical composition further comprises an additional therapeutic agent selected from the group consisting of alkylating agents, angiogenesis inhibitors, antibodies, antimetabolites, antimitotics, antiproliferatives, antivirals, aurora kinase inhibitors, apoptosis promoters, activators of death receptor pathway, Bcr-Abl kinase inhibitors, BiTE (Bi-Specific T cell Engager) antibodies, antibody drug conjugates, biologic response modifiers, Bruton's tyrosine kinase (BTK) inhibitors, cyclin-dependent kinase inhibitors, cell cycle inhibitors, cyclooxygenase-2 inhibitors, DVDs, leukemia viral oncogene homolog (ErbB2) receptor inhibitors, growth factor inhibitors, heat shock protein (HSP)-90 inhibitors, histone deacetylase (HDAC) inhibitors, hormonal therapies, immunologicals, inhibitors of apoptosis proteins (IAPs), intercalating antibiotics, kinase inhibitors, kinesin inhibitors, Jak2 inhibitors, mammalian target of rapamycin inhibitors, microRNAs, mitogen-activated extracellular signal-regulated kinase inhibitors, multivalent binding proteins, non-steroidal anti-inflammatory drugs (NSAIDs), poly ADP (adenosine diphosphate)-ribose polymerase (PARP) inhibitors, platinum chemotherapeutics, polo-like kinase (Plk) inhibitors, phosphoinositide-3 kinase (PI3K) inhibitors, proteasome inhibitors, purine analogs, pyrimidine analogs, receptor tyrosine kinase inhibitors, retinoids/deltoids plant alkaloids, small inhibitory ribonucleic acids (siRNAs), topoisomerase inhibitors, ubiquitin ligase inhibitors, hypomethylating agents, checkpoints inhibitors, peptide vaccines, epitopes or neoepitopes from tumor antigens, and combinations of one or more of these agents.

39. A method of treating cancer in a subject comprising the administration of a composition according to claim 37 to a subject in need of treatment.

40. The method of claim 39, wherein the cancer is selected from the group consisting of a hematologic malignancy or a solid tumor with expression of PD-1 and/or PD-L1, selected from hematolymphoid neoplasms, angioimmunoblastic T cell lymphoma, myelodysplasic syndrome, and acute myeloid leukemia, a cancer induced by virus or associated with immunodeficiency, Kaposi sarcoma, cervical, anal, penile and vulvar squamous cell cancer, oropharyndeal cancers, B cell non-Hodgkin lymphomas (NHL), diffuse large B-cell lymphoma, Burkitt lymphoma, plasmablastic lymphoma, primary central nervous system lymphoma, HHV-8 primary effusion lymphoma, classic Hodgkin lymphoma, lymphoproliferative disorders, hepatocellular carcinoma, Merkel cell carcinoma, cancer associated with human immunodeficiency virus infection (HIV) infection, metastatic or non-metastatic cancer, melanoma, malignant mesothelioma, Non-Small Cell Lung Cancer, Renal Cell Carcinoma, Hodgkin's Lymphoma, Head and Neck Cancer, Urothelial Carcinoma, Colorectal Cancer, Hepatocellular Carcinoma, Small Cell Lung Cancer, Metastatic Merkel Cell Carcinoma, Gastric or Gastroesophageal cancers and Cervical Cancer.

41. The method of claim 39, wherein said pharmaceutical composition is administered in combination with radiotherapy or an additional therapeutic agent selected from the group consisting of alkylating agents, angiogenesis inhibitors, antibodies, antimetabolites, antimitotics, antiproliferatives, antivirals, aurora kinase inhibitors, apoptosis promoters, activators of death receptor pathway, Bcr-Abl kinase inhibitors, BiTE (Bi-Specific T cell Engager) antibodies, antibody drug conjugates, biologic response modifiers, Bruton's tyrosine kinase (BTK) inhibitors, cyclin-dependent kinase inhibitors, cell cycle inhibitors, cyclooxygenase-2 inhibitors, DVDs, leukemia viral oncogene homolog (ErbB2) receptor inhibitors, growth factor inhibitors, heat shock protein (HSP)-90 inhibitors, histone deacetylase (HDAC) inhibitors, hormonal therapies, immunologicals, inhibitors of apoptosis proteins (IAPs), intercalating antibiotics, kinase inhibitors, kinesin inhibitors, Jak2 inhibitors, mammalian target of rapamycin inhibitors, microRNAs, mitogen-activated extracellular signal-regulated kinase inhibitors, multivalent binding proteins, non-steroidal anti-inflammatory drugs (NSAIDs), poly ADP (adenosine diphosphate)-ribose polymerase (PARP) inhibitors, platinum chemotherapeutics, polo-like kinase (Plk) inhibitors, phosphoinositide-3 kinase (PI3K) inhibitors, proteasome inhibitors, purine analogs, pyrimidine analogs, receptor tyrosine kinase inhibitors, retinoids/deltoids plant alkaloids, small inhibitory ribonucleic acids (siRNAs), topoisomerase inhibitors, ubiquitin ligase inhibitors, hypomethylating agents, checkpoints inhibitors, peptide vaccines, epitopes or neoepitopes from tumor antigens, and combinations of one or more of these agents.

42. A method of treating an infectious disease, a chronic infectious disease, or chronic viral infections comprising the administration of a composition according to claim 37 to a subject in need of treatment.

43. The method of claim 42, wherein the infectious disease is caused by a virus selected from the group consisting of HIV, hepatitis virus, herpes virus, adenovirus, influenza virus, flaviviruses, echovirus, rhinovirus, coxsackie virus, coronavirus, respiratory syncytial virus, mumps virus, rotavirus, measles virus, rubella virus, parvovirus, vaccinia virus, HTLV virus, dengue virus, papillomavirus, molluscum virus, poliovirus, rabies virus, JC virus and arboviral encephalitis virus.


SEQUENCE LISTING (56 sequences)

SEQ ID NO: 1 - protein, 5 aa, artificial
  HC CDR1
HYAMN

SEQ ID NO: 2 - protein, 17 aa, artificial
  HC CDR2
WINTNTGEPTYAQGFTG

SEQ ID NO: 3 - protein, 8 aa, artificial
  HC CDR3
  MISC_FEATURE (7)..(7): X is X1 (D or E)
  MISC_FEATURE (8)..(8): X is X2 and is T, H, A, Y, N, E or S
EREPGMXX

SEQ ID NO: 4 - protein, 8 aa, artificial
  HC CDR3
EREPGMDS

SEQ ID NO: 5 - protein, 8 aa, artificial
  HC CDR3
EREPGMDT

SEQ ID NO: 6 - protein, 8 aa, artificial
  HC CDR3
EREPGMES

SEQ ID NO: 7 - protein, 8 aa, artificial
  HC CDR3
EREPGMDH

SEQ ID NO: 8 - protein, 8 aa, artificial
  HC CDR3
EREPGMDA

SEQ ID NO: 9 - protein, 8 aa, artificial
  HC CDR3
EREPGMDY

SEQ ID NO: 10 - protein, 8 aa, artificial
  HC CDR3
EREPGMDN

SEQ ID NO: 11 - protein, 8 aa, artificial
  HC CDR3
EREPGMDE

SEQ ID NO: 12 - protein, 16 aa, artificial
  LC CDR1
  MISC_FEATURE (11)..(11): X is G or T
RSSQSLVHANXNTYLE

SEQ ID NO: 13 - protein, 16 aa, artificial
  LC CDR1
RSSQSLVHANGNTYLE

SEQ ID NO: 14 - protein, 16 aa, artificial
  LC CDR1
RSSQSLVHANTNTYLE

SEQ ID NO: 15 - protein, 7 aa, artificial
  LC CDR2
KVSNRFS

SEQ ID NO: 16 - protein, 9 aa, artificial
  LC CDR3
FQGTHVPNT

SEQ ID NO: 17 - protein, 117 aa, artificial
  HC variable domain
  MISC_FEATURE (105)..(105): X is X1, D or E
  MISC_FEATURE (106)..(106): X is X2, and is T, H, A, Y, N, E or S
QIQLVQSGSELKKPGASVKVSCKASGYTFTHYAMNWVRQAPGQGLEWMGWINTNTGEPTYAQGFTGRFVFSLDTSVSTAYLQISSLKAEDTAVYYCAREREPGMXXWGQGTLVTVSS

SEQ ID NO: 18 - protein, 117 aa, artificial
  HC variable domain
QIQLVQSGSELKKPGASVKVSCKASGYTFTHYAMNWVRQAPGQGLEWMGWINTNTGEPTYAQGFTGRFVFSLDTSVSTAYLQISSLKAEDTAVYYCAREREPGMDSWGQGTLVTVSS

SEQ ID NO: 19 - protein, 117 aa, artificial
  HC variable domain
QIQLVQSGSELKKPGASVKVSCKASGYTFTHYAMNWVRQAPGQGLEWMGWINTNTGEPTYAQGFTGRFVFSLDTSVSTAYLQISSLKAEDTAVYYCAREREPGMDTWGQGTLVTVSS

SEQ ID NO: 20 - protein, 117 aa, artificial
  HC variable domain
QIQLVQSGSELKKPGASVKVSCKASGYTFTHYAMNWVRQAPGQGLEWMGWINTNTGEPTYAQGFTGRFVFSLDTSVSTAYLQISSLKAEDTAVYYCAREREPGMESWGQGTLVTVSS

SEQ ID NO: 21 - protein, 117 aa, artificial
  HC variable domain
QIQLVQSGSELKKPGASVKVSCKASGYTFTHYAMNWVRQAPGQGLEWMGWINTNTGEPTYAQGFTGRFVFSLDTSVSTAYLQISSLKAEDTAVYYCAREREPGMDHWGQGTLVTVSS

SEQ ID NO: 22 - protein, 117 aa, artificial
  HC variable domain
QIQLVQSGSELKKPGASVKVSCKASGYTFTHYAMNWVRQAPGQGLEWMGWINTNTGEPTYAQGFTGRFVFSLDTSVSTAYLQISSLKAEDTAVYYCAREREPGMDAWGQGTLVTVSS

SEQ ID NO: 23 - protein, 117 aa, artificial
  HC variable domain
QIQLVQSGSELKKPGASVKVSCKASGYTFTHYAMNWVRQAPGQGLEWMGWINTNTGEPTYAQGFTGRFVFSLDTSVSTAYLQISSLKAEDTAVYYCAREREPGMDYWGQGTLVTVSS

SEQ ID NO: 24 - protein, 117 aa, artificial
  HC variable domain
QIQLVQSGSELKKPGASVKVSCKASGYTFTHYAMNWVRQAPGQGLEWMGWINTNTGEPTYAQGFTGRFVFSLDTSVSTAYLQISSLKAEDTAVYYCAREREPGMDNWGQGTLVTVSS

SEQ ID NO: 25 - protein, 117 aa, artificial
  HC variable domain
QIQLVQSGSELKKPGASVKVSCKASGYTFTHYAMNWVRQAPGQGLEWMGWINTNTGEPTYAQGFTGRFVFSLDTSVSTAYLQISSLKAEDTAVYYCAREREPGMDEWGQGTLVTVSS

SEQ ID NO: 26 - protein, 112 aa, artificial
  LC variable domain
  MISC_FEATURE (34)..(34): X is G or T
DVVMTQSPLSLPVTLGQPASISCRSSQSLVHANXNTYLEWYQQRPGQSPRLLIYKVSNRFSGVPDRFSGSGSGTDFTLKISRVEAEDVGVYYCFQGTHVPNTFGQGTKLEIK

SEQ ID NO: 27 - protein, 112 aa, artificial
  LC variable domain
DVVMTQSPLSLPVTLGQPASISCRSSQSLVHANGNTYLEWYQQRPGQSPRLLIYKVSNRFSGVPDRFSGSGSGTDFTLKISRVEAEDVGVYYCFQGTHVPNTFGQGTKLEIK

SEQ ID NO: 28 - protein, 112 aa, artificial
  LC variable domain
DVVMTQSPLSLPVTLGQPASISCRSSQSLVHANTNTYLEWYQQRPGQSPRLLIYKVSNRFSGVPDRFSGSGSGTDFTLKISRVEAEDVGVYYCFQGTHVPNTFGQGTKLEIK

SEQ ID NO: 29 - protein, 444 aa, artificial
  HC
QIQLVQSGSELKKPGASVKVSCKASGYTFTHYAMNWVRQAPGQGLEWMGWINTNTGEPTYAQGFTGRFVFSLDTSVSTAYLQISSLKAEDTAVYYCAREREPGMDSWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSPGK

SEQ ID NO: 30 - protein, 444 aa, artificial
  HC
QIQLVQSGSELKKPGASVKVSCKASGYTFTHYAMNWVRQAPGQGLEWMGWINTNTGEPTYAQGFTGRFVFSLDTSVSTAYLQISSLKAEDTAVYYCAREREPGMDTWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSPGK

SEQ ID NO: 31 - protein, 444 aa, artificial
  HC
QIQLVQSGSELKKPGASVKVSCKASGYTFTHYAMNWVRQAPGQGLEWMGWINTNTGEPTYAQGFTGRFVFSLDTSVSTAYLQISSLKAEDTAVYYCAREREPGMESWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSPGK

SEQ ID NO: 32 - protein, 444 aa, artificial
  HC
QIQLVQSGSELKKPGASVKVSCKASGYTFTHYAMNWVRQAPGQGLEWMGWINTNTGEPTYAQGFTGRFVFSLDTSVSTAYLQISSLKAEDTAVYYCAREREPGMDHWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSPGK

SEQ ID NO: 33 - protein, 444 aa, artificial
  HC
QIQLVQSGSELKKPGASVKVSCKASGYTFTHYAMNWVRQAPGQGLEWMGWINTNTGEPTYAQGFTGRFVFSLDTSVSTAYLQISSLKAEDTAVYYCAREREPGMDAWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSPGK

SEQ ID NO: 34 - protein, 444 aa, artificial
  HC
QIQLVQSGSELKKPGASVKVSCKASGYTFTHYAMNWVRQAPGQGLEWMGWINTNTGEPTYAQGFTGRFVFSLDTSVSTAYLQISSLKAEDTAVYYCAREREPGMDYWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSPGK

SEQ ID NO: 35 - protein, 444 aa, artificial
  HC
QIQLVQSGSELKKPGASVKVSCKASGYTFTHYAMNWVRQAPGQGLEWMGWINTNTGEPTYAQGFTGRFVFSLDTSVSTAYLQISSLKAEDTAVYYCAREREPGMDNWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSPGK

SEQ ID NO: 36 - protein, 444 aa, artificial
  HC
QIQLVQSGSELKKPGASVKVSCKASGYTFTHYAMNWVRQAPGQGLEWMGWINTNTGEPTYAQGFTGRFVFSLDTSVSTAYLQISSLKAEDTAVYYCAREREPGMDEWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSPGK

SEQ ID NO: 37 - protein, 219 aa, artificial
  LC
DVVMTQSPLSLPVTLGQPASISCRSSQSLVHANGNTYLEWYQQRPGQSPRLLIYKVSNRFSGVPDRFSGSGSGTDFTLKISRVEAEDVGVYYCFQGTHVPNTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 38 - protein, 219 aa, artificial
  LC
DVVMTQSPLSLPVTLGQPASISCRSSQSLVHANTNTYLEWYQQRPGQSPRLLIYKVSNRFSGVPDRFSGSGSGTDFTLKISRVEAEDVGVYYCFQGTHVPNTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 39 - protein, 327 aa, artificial
  HC constant domain
ASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSPGK

SEQ ID NO: 40 - protein, 107 aa, artificial [sequence omitted]

SEQ ID NO: 41 - protein, 30 aa, artificial
  HFR1
QIQLVQSGSELKKPGASVKVSCKASGYTFT

SEQ ID NO: 42 - protein, 14 aa, artificial
  HFR2
WVRQAPGQGLEWMG

SEQ ID NO: 43 - protein, 32 aa, artificial
  HFR3
RFVFSLDTSVSTAYLQISSLKAEDTAVYYCAR

SEQ ID NO: 44 - protein, 11 aa, artificial
  HFR4
WGQGTLVTVSS

SEQ ID NO: 45 - protein, 23 aa, artificial
  LFR1
DVVMTQSPLSLPVTLGQPASISC

SEQ ID NO: 46 - protein, 15 aa, artificial
  LFR2
WYQQRPGQSPRLLIY

SEQ ID NO: 47 - protein, 32 aa, artificial
  LFR3
GVPDRFSGSGSGTDFTLKISRVEAEDVGVYYC

SEQ ID NO: 48 - protein, 10 aa, artificial
  LFR4
FGQGTKLEIK

SEQ ID NO: 49 - protein, 19 aa, artificial
  signal peptide
MDWLWNLLFLMAAAQSIQA

SEQ ID NO: 50 - protein, 19 aa, artificial
  signal peptide
MKLPVRLLVLMFWIPASSS

SEQ ID NO: 51 - protein, 343 aa, artificial
  SIRPa
EEELQVIQPDKSVLVAAGETATLRCTATSLIPVGPIQWFRGAGPGRELIYNQKEGHFPRVTTVSDLTKRNNMDFSIRIGNITPADAGTYYCVKFRKGSPDDVEFKSGAGTELSVRAKPSAPVVSGPAARATPQHTVSFTCESHGFSPRDITLKWFKNGNELSDFQTNVDPVGESVSYSIHSTAKVVLTREDVHSQVICEVAHVTLQGDPLRGTANLSETIRVPPTLEVTQQPVRAENQVNVTCQVRKFYPQRLQLTWLENGNVSRTETASTVTENKDGTYNWMSWLLVNVSAHRDDVKLTCQVEHDGQPAVSKSHDLKVSAHPKEQGSNTAAENTGSNERNIY

SEQ ID NO: 52 - protein, 330 aa, artificial
  Heavy chain constant domain (IgG1m-N298A)
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYASTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 53 - protein, 444 aa, artificial
  HC anti-PD1 chimeric
QIQLVQSGPELKKPGETVKISCKASGYTFTHYGMNWVKQAPGKGLKWMGWINTNTGEPTYAEEFKGRFAFSLETSASAAFLQINNLKNEDTATYFCAKEREPGMDSWGQGTSVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSPGK

SEQ ID NO: 54 - protein, 219 aa, artificial
  LC anti-PD1 chimeric
DVLLTQTPLSLPVSLGDQASISCRSSQNIVHANGNTYLEWYLQKPGQSPKLLIYKVSNRFSGVPDRFSGSGSGTDFTLKITRVEAEDLGVYYCFQGSHVPNTFGGGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 55 - DNA, 2670 bp, artificial
  VH-VK
cagatccagctggtgcagagcggctctgagctgaagaagccaggcgcttctgtgaaggtgtcctgcaaggccagcggctacaccttcacacactatgctatgaattgggtgagacaggctccaggacagggactggagtggatgggctggatcaacaccaatacaggcgagcctacctacgctcagggctttacaggccgcttcgtgttttctctggatacctccgtgagcacagcctatctgcagatctccagcctgaaggctgaggacaccgccgtgtactattgtgctagggagagggagccaggaatggataactggggacagggcaccctggtgacagtgtcttccgctagcaccaagggcccatcggtcttccccctggcgccctgctccaggagcacctccgagagcacagccgccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacgaagacctacacctgcaacgtagatcacaagcccagcaacaccaaggtggacaagagagttgagtccaaatatggtcccccatgcccaccatgcccagcacctgagttcctggggggaccatcagtcttcctgttccccccaaaacccaaggacactctcatgatctcccggacccctgaggtcacgtgcgtggtggtggacgtgagccaggaagaccccgaggtccagttcaactggtacgtggatggcgtggaggtgcataatgccaagacaaagccgcgggaggagcagttcaacagcacgtaccgtgtggtcagcgtcctcaccgtcctgcaccaggactggctgaacggcaaggagtacaagtgcaaggtctccaacaaaggcctcccgtcctccatcgagaaaaccatctccaaagccaaagggcagccccgagagccacaggtgtacaccctgcccccatcccaggaggagatgaccaagaaccaggtcagcctgacctgcctggtcaaaggcttctaccccagcgacatcgccgtggagtgggagagcaatgggcagccggagaacaactacaagaccacgcctcccgtgctggactccgacggctccttcttcctctacagcaggctaaccgtggacaagagcaggtggcaggaggggaatgtcttctcatgctccgtgatgcatgaggctctgcacaaccactacacacagaagagcctctccctgtctccgggtaaatgacagatccagctggtgcagagcggctctgagctgaagaagccaggcgcttctgtgaaggtgtcctgcaaggccagcggctacaccttcacacactatgctatgaattgggtgagacaggctccaggacagggactggagtggatgggctggatcaacaccaatacaggcgagcctacctacgctcagggctttacaggccgcttcgtgttttctctggatacctccgtgagcacagcctatctgcagatctccagcctgaaggctgaggacaccgccgtgtactattgtgctagggagagggagccaggaatggataactggggacagggcaccctggtgacagtgtcttccgctagcaccaagggcccatcggtcttccccctggcgccctgctccaggagcacctccgagagcacagccgccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacgaagacctacacctgcaacgtagatcacaagcccagcaacaccaaggtggacaagagagttgagtccaaatatggtcccccatgcccaccatgcccagcacctgagttcctggggggaccatcagtcttcctgttccccccaaaacccaaggacactctcatgatctcccggacccctgaggtcacgtgcgtggtggtggacgtgagccaggaagaccccgaggtccagttcaactggtacgtggatggcgtggaggtgcataatgccaagacaaagccgcgggaggagcagttcaacagcacgtaccgtgtggtcagcgtcctcaccgtcctgcaccaggactggctgaacggcaaggagtacaagtgcaaggtctccaacaaaggcctcccgtcctccatcgagaaaaccatctccaaagccaaagggcagccccgagagccacaggtgtacaccctgcccccatcccaggaggagatgaccaagaaccaggtcagcctgacctgcctggtcaaaggcttctaccccagcgacatcgccgtggagtgggagagcaatgggcagccggagaacaactacaagaccacgcctcccgtgctggactccgacggctccttcttcctctacagcaggctaaccgtggacaagagcaggtggcaggaggggaatgtcttctcatgctccgtgatgcatgaggctctgcacaaccactacacacagaagagcctctccctgtctccgggtaaatga

SEQ ID NO: 56 - DNA, 660 bp, artificial
  VL-VD
gacgtggtcatgacacagagcccactgtctctgcctgtgaccctgggacagccagcctctatctcctgcagatccagccagtctctggtgcacgctaacaccaatacatacctggagtggtatcagcagaggccaggacagtccccaaggctgctgatctacaaggtgtccaacagattcagcggagtgccagaccgctttagcggatctggatccggaaccgacttcaccctgaagatctccagggtggaggctgaggatgtgggcgtgtactattgtttccagggcacccatgtgcctaatacatttggccagggcaccaagctggagatcaagcgtacggtggctgcaccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgagctcgcccgtcacaaagagcttcaacaggggagagtgttag